(19) United States
(12) Patent Application Publication
(10) Pub. No.: US 2026/0157800 A1
(43) Pub. Date: Jun. 11, 2026

(54) Apparatus and Method for Skin Graft Expansion in Split-Thickness Skin Graft Surgeries

(71) Applicants: New Jersey Institute of Technology, Newark, NJ (US); Washington University, St. Louis, MO (US)

(72) Inventors: Farid Alisafaei, Newark, NJ (US); Guy Genin, St. Louis, MO (US); Mohammad Jafari, Newark, NJ (US); Aliza Mujahid, Newark, NJ (US); Haomin Yu, St. Louis, MO (US)

(73) Assignees: New Jersey Institute of Technology, Newark, NJ (US); Washington University, St. Louis, MO (US)

(21) Appl. No.: 19/410,888

(22) Filed: Dec. 5, 2025

Related U.S. Application Data
(60) Provisional application No. 63/728,300, filed on Dec. 5, 2024.

(51) Int. Cl. A61B 34/10; A61B 17/00; A61B 17/32; A61B 17/322; A61B 90/00

(52) U.S. Cl. CPC A61B 34/10; A61B 17/32; A61B 90/06; A61B 2017/00119; A61B 2017/00128; A61B 2017/00137; A61B 2017/3225; A61B 2034/108; A61B 2090/064

(57) ABSTRACT

A method for optimizing skin graft expansion in split-thickness skin graft surgeries includes selecting, from an integrated system, a desired expansion ratio for a specific skin graft expansion in a patient; receiving, by the integrated system, an input of a set of geometric constraints; determining, by the integrated system, a first set of cutting patterns based on the desired expansion ratio and the input set of geometric constraints, wherein the first set of cutting patterns satisfies both the desired expansion ratio and the set of geometric constraints; and outputting, by the integrated system, the first set of cutting patterns, the first set of cutting patterns including a cutting pattern including one or more of a parallel cutting pattern or a complex geometrical cutting pattern.

### CROSS-REFERENCE TO RELATED APPLICATIONS

The present application claims the benefit of the filing date of U.S. Provisional Application No. 63/728,300 filed Dec. 5, 2024, the disclosure of which is hereby incorporated herein by reference.

### GOVERNMENT STATEMENT

This invention was made with government support under R01AR084243 and AR077793 awarded by the National Institutes of Health and CMMI1548571 awarded by the National Science Foundation. The government has certain rights in the invention.

### FIELD OF USE

The present disclosure relates to advancement in techniques of meshing skin grafts to improve outcomes of skin graft surgeries. More particularly, it relates to a process and system for optimizing skin graft expansion in split-thickness skin graft surgeries. An associated device component includes a smart meshing tool with interchangeable cutting heads capable of both conventional parallel cutting and advanced patterns, and integrates with software that guides cutting. Such software could be integrated with the system. The process aspect involves a method for predicting and achieving optimal graft expansion ratios, and may also include incorporating real-time skin property assessment, adaptive cutting patterns, and a predictive model that accounts for both mechanical principles and surgical intuition. The disclosed process and system, as well as the integrated device, either alone or in combination, improve the accuracy, efficiency, and outcomes of skin graft procedures.

### BACKGROUND

It is well known that the skin is the largest organ of the human body and accounts for about 16% of the total human weight. Because skin forms the interface with the environment, it plays an important role in biodefense and serves as an anatomical barrier from pathogens and other environmental substances. The skin also provides a semipermeable barrier to prevent excessive fluid loss while ensuring that essential nutrients are not washed away from the body. Other functions of the skin include, for example, isolation, thermoregulation, and sensation. Skin tissue however can suffer many forms of damage, including burns, trauma, illness, and depigmentation.

Often skin grafts are used to repair such skin damage. Skin grafts involve cutting a piece of skin from an area of the human body (autograft), from another person (allograft), or from another animal (xenograft), and surgery for transplantation at a transplant site, such as a wound site. As with any surgical procedure, skin transplantation involves certain risks. Complications can include, for example, transplant failure; skin graft rejection; infection of the transplant site; or exudation of fluid and blood from the graft during healing. Some of these complications may be alleviated by using autografts instead of allografts or xenografts. A problem faced when using autografts is the creation of trauma and wounds to remove skin from another area of the human body to produce the graft. Generally, since the size of the graft matches the size of the transplanted site, if the transplanted site is large, it is necessary to cut out a large skin piece from the donor site.

As the size of the skin piece cut from the donor site increases, the probability that the donor site will not heal properly increases, requiring additional treatment and intervention. Furthermore, the probability of infection increases as the size of the skin pieces cut from the donor site increases. Due to the large wounds, the healing time associated with the removal of large pieces of skin also increases.

In order to address these problems, techniques have been developed to expand skin grafts so that the harvested graft can treat a transplant site larger than the donor site. Some of these techniques include harvesting the graft and then culturing the graft in the presence of growth factors and other biological products that grow the skin graft. Another method involves adding trypsin to the graft to produce a graft piece that is mixed into the transplant site. However, problems associated with the use of other biological products such as growth factors and trypsin have been attributed to possible side effects such as cancer in transplanted cells, where such substances are implicated in unregulated cell proliferation.

In addition, current skin graft meshing devices used in surgeries often fail to achieve the intended graft expansion ratio. This discrepancy between the claimed ratio by the manufacturer and the actual ratio achieved in surgery leads to several issues. For example, unreliable wound coverage, where surgeons cannot accurately predict how much a skin graft will expand, making it difficult to ensure sufficient coverage for large wounds. Another issue is excess donor site trauma. To compensate for the unreliable expansion, surgeons may harvest more skin than necessary from healthy donor sites, increasing patient discomfort and healing time.

Yet another issue is overestimation by surgeons. Even with awareness of the discrepancy, surgeons tend to significantly overestimate the actual achievable expansion ratio. Such overestimation leads to significant consequences such as in major burns, underestimating donor requirements may necessitate additional procedures, while in elderly or healing-compromised patients, failure to minimize incision burden may prolong recovery.

Conventional meshing devices typically use a simple parallel cutting pattern to create slits in the skin graft. They are characterized by fixed cutting patterns with limited variability. The mechanical operation is without integrated sensing or feedback mechanisms and typically has predefined expansion ratios. Conventional models for expansion ratio used to estimate the nominal expansion ratio (Rn) assumes each slit transforms into a perfect square when expanded. Of course, such expansion into a perfect square is rarely achieved in skin grafts. In some cases, surgeons may create meshing patterns manually using surgical instruments, especially for small grafts or specific requirements. This manual technique can lead to several inconsistencies. A technique known commonly as the Meek Technique is an alternative method for skin graft expansion that involves cutting the graft into small squares and spreading them apart on a carrier material, however, this technique may also lead to negative issues. These methods and devices are characterized by limited predictability and consistency in achieving desired expansion ratios, lack of real-time adaptation to specific skin properties, absence of integrated sensing and feedback mechanisms, reliance on simple, fixed cutting patterns, and disconnection between theoretical models and actual clinical outcomes. Existing mechanical models that can accurately predict actual expansion from meshing parameters require computational implementation that limit clinical uptake.

### SUMMARY

The present methods and devices ensure reliable wound coverage, minimize donor site trauma, enable dual-directional expansion, and reduce tension within grafts to prevent fibroblast activation. The innovation can surpass the current standard by consistently achieving the claimed expansion ratio. This eliminates the issue of unreliable wound coverage and reduces the risk of surgeons over-harvesting from donor sites. The present method identifies feasible meshing configurations for target expansion ratios enabling surgeons to optimize for either maximum expansion efficiency or minimum incision burden based on patient-specific priorities without the need for the surgeon to perform computations.

The present methods and devices allow enabling higher expansion ratios with fewer and shorter cuts. This minimizes trauma to the patient by requiring less healthy skin to be harvested, potentially leading to faster healing times at the donor site. Another significant issue in split-thickness skin grafting is the high rate of graft failure, with one of the primary causes being “Secondary Contracture.” The present methods and devices have recently demonstrated that one important factor contributing to the activation of dermal fibroblasts, and consequently their heightened contractility, is the level of mechanical tension they experience within the graft.

The present disclosure addresses these limitations by introducing a more sophisticated, adaptive, and predictive approach to skin graft meshing in several keyways unlike the conventional systems that have several drawbacks. Conventional meshing devices rely on parallel cut patterns, leading to unreliable expansion ratios. Achieved expansion often falls short of the claimed ratio by the manufacturer. Excess donor site trauma may occur as surgeons may over-harvest skin to compensate for unreliable expansion, increasing patient discomfort and healing time. In addition, the surgeons have limited control over design, the design does not account for the mechanical properties of skin, hindering precise control over expansion.

The present disclosure offers both conventional parallel cutting and kirigami-inspired (using geometric cuts identical, analogous to, or inspired by the Japanese art of paper cutting to create desired mechanical properties) design modes. The enhanced conventional mode based on parallel or similar cuts delivers an improved expansion ratio compared to current state-of-the-art while using fewer and shorter cuts for reducing donor site trauma. This caters to surgeons comfortable with a familiar approach. The kirigami-inspired mode or using geometric cuts based on the Japanese art of paper cutting to create precise mechanical properties uses metamaterial analysis to create intricate cut patterns. This offers significant advantages over the current state of the art. There are significantly higher expansion ratios with high precision thereby eliminating under- or over-expansion issues.

In one aspect, a method for optimizing skin graft expansion in split-thickness skin graft surgeries includes selecting, from an integrated system, a desired expansion ratio for a specific skin graft expansion in a patient; receiving, by the integrated system, an input of a set of geometric constraints; determining, by the integrated system, a first set of cutting patterns based on the desired expansion ratio and the input set of geometric constraints, wherein the first set of cutting patterns satisfies both the desired expansion ratio and the set of geometric constraints; and outputting, by the integrated system, the first set of cutting patterns, the first set of cutting patterns including a cutting pattern including one or more of a parallel cutting pattern or a complex geometrical cutting pattern.

In some aspects, the determining of the first set of cutting patterns includes selecting the first set of cutting patterns from a larger set of cutting patterns stored in the integrated system, the selecting of the first set of cutting patterns being based on which of the larger set of cutting patterns satisfies both the desired expansion ratio and the set of geometric constraints.

In some aspects, the larger set of cutting patterns are selected prior to the selecting of the desired expansion ratio. In some aspects, the larger set of cutting patterns are stored in a memory of the integrated system.

In some aspects, the larger set of cutting patterns are selected by excluding possible cutting patterns that: result in geometric overlap between adjacent cuts, exceed predefined limits on spacing between cuts, have fewer than a minimum number of cuts in a horizontal and a vertical direction, and are unable to achieve the desired expansion ratio.

In some aspects, the geometric overlap between adjacent cuts is determined using a cut length L, a horizontal spacing between cuts d, and a vertical gap between cut ends g.

In some aspects, the selecting of the desired expansion ratio R from the integrated system for the specific skin graft expansion in the patient comprises determining R using the following Eq. 2:

$$R = \frac{1}{2} ( \frac{1 + λ}{1 + γ} ) [ 2 γ + \sqrt{{( 1 - γ )}^{2} - {ϕ ( λ - 1 )}^{2} {η}^{2}} ] \qquad ( 2 )$$

wherein λ=1+(δ/d), γ=g/L, and η=d/L.

In some aspects, an upper limit for the extension ratio R_{U} is determined using the following Eq. 3:

$${R}_{U} = \frac{1}{2} ( 1 + λ ) \qquad ( 3 )$$

In some aspects, a lower limit for the extension ratio R_{L} is determined using the following Eq. 4:

$${R}_{L} = \{ \begin{matrix}\frac{1}{2} ( \frac{1 + λ}{1 + γ} ) [ 2 γ + \sqrt{{( 1 - γ )}^{2} - {( λ - 1 )}^{2} {η}^{2}} ] , & λ \leq {λ}_{c} \\ \frac{γ ( 1 + λ )}{1 + γ} , & λ > {λ}_{c}\end{matrix} \qquad ( 4 )$$

In some aspects, a limit of elongation λₐ is determined using the following Eq. 5:

$${λ}_{a} = 1 + \frac{k}{η} \sqrt{2 ( 1 - {γ}^{2} ) - {{k}^{2} ( 1 + γ )}^{2}} \qquad ( 5 )$$

wherein R_{U}−R_{L}=k²R_{U}.

In some aspects, an actual expansion ratio Rₐ is determined using the following Eq.6:

$${R}_{a} = \frac{1}{2} ( 1 + {λ}_{a} ) \qquad ( 6 )$$

In some aspects, the method further comprises initiating a cutting process with a device, wherein the device comprises an adaptive cutting mechanism having a multi-modal cutting head and a force-feedback sensor integrated into the cutting mechanism to monitor a resistance during cutting for real-time adjustments to a cutting depth and a cutting speed.

In some aspects, the method further comprises placing a harvested graft in the device, wherein the device further comprises a microfluidic tissue support that uses micro-fluidics to maintain a specific position and a tension of the harvested graft during the cutting process of the parallel cutting pattern or the complex geometrical cutting pattern without damaging tissue.

In some aspects, the method further comprises expanding the harvested graft and receiving from the integrated system a real-time feedback on achieved expansion ratio as the harvested graft is expanded.

In some aspects, the real-time feedback on the achieved expansion ratio further comprises providing an alert when the harvested graft approaches a selected expansion point, and wherein the selected expansion point is based on a predictive model and sensor data.

In some aspects, the method further comprises logging data including parameters recorded by the integrated system of a skin grafting procedure for future reference and analysis.

In some aspects, the method further comprises conducting a post-procedure analysis wherein the integrated system provides a summary of a skin grafting procedure including an actual expansion ratio and deviations from predictions.

In some aspects, the complex geometric cutting pattern includes kirigami-inspired cuts.

In some aspects, the method further comprises adjusting the device in real-time using the resistance monitored by the force-feedback sensor during the cutting process.

In some aspects, the adjustment of the device further comprises allowing the integrated system to adjust the cutting depth and the cutting speed in real-time.

In another aspect, the present disclosure relates to a non-transitory computer readable medium storing instructions for skin graft expansion in split-thickness skin graft surgeries that, when executed by one or more processors, cause the one or more processors to: select, from an integrated system, a desired expansion ratio for a specific skin graft expansion in a patient; receive, by the integrated system, an input of a set of geometric constraints; determine, by the integrated system, a first set of cutting patterns based on the desired expansion ratio and the input set of geometric constraints, wherein the first set of cutting patterns satisfies both the desired expansion ratio and the set of geometric constraints; and output, by the integrated system, the first set of cutting patterns, the first set of cutting patterns including a cutting pattern including one or more of a parallel cutting pattern or a complex geometrical cutting pattern.

In another aspect, a system for optimizing skin graft expansion in split-thickness skin graft surgeries includes a device; and one or more processors in communication with the device, the one or more processors configured to: select, from an integrated system, a desired expansion ratio for a specific skin graft expansion in a patient; receive, by the integrated system, an input of a set of geometric constraints; determine, by the integrated system, a first set of cutting patterns based on the desired expansion ratio and the input set of geometric constraints, wherein the first set of cutting patterns satisfies both the desired expansion ratio and the set of geometric constraints; and output, by the integrated system, the first set of cutting patterns, the first set of cutting patterns including a cutting pattern including one or more of a parallel cutting pattern or a complex geometrical cutting pattern.

In some aspects, the determining of the first set of cutting patterns includes selecting the first set of cutting patterns from a larger set of cutting patterns stored in the integrated system, the selecting of the first set of cutting patterns being based on which of the larger set of cutting patterns satisfies both the desired expansion ratio and the set of geometric constraints.

In some aspects, the larger set of cutting patterns are selected prior to the selecting of the desired expansion ratio. In some aspects, the larger set of cutting patterns are stored in a memory of the integrated system.

In some aspects, the larger set of cutting patterns are selected by excluding possible cutting patterns that: result in geometric overlap between adjacent cuts, exceed predefined limits on spacing between cuts, have fewer than a minimum number of cuts in a horizontal and a vertical direction, and are unable to achieve the desired expansion ratio.

In some aspects, the geometric overlap between adjacent cuts is determined using a cut length L, a horizontal spacing between cuts d, and a vertical gap between cut ends g.

In some aspects, the selecting of the desired expansion ratio R from the integrated system for the specific skin graft expansion in the patient comprises determining R using the following Eq. 2:

$$R = \frac{1}{2} ( \frac{1 + λ}{1 + γ} ) [ 2 γ + \sqrt{{( 1 - γ )}^{2} - {ϕ ( λ - 1 )}^{2} {η}^{2}} ] \qquad ( 2 )$$

wherein λ=1+(δ/d), γ=g/L, and η=d/L.

In some aspects, an upper limit for the extension ratio R_{U} is determined using the following Eq. 3:

$${R}_{U} = \frac{1}{2} ( 1 + λ ) \qquad ( 3 )$$

In some aspects, a lower limit for the extension ratio R_{L} is determined using the following Eq. 4:

$${R}_{L} = \{ \begin{matrix}\frac{1}{2} ( \frac{1 + λ}{1 + γ} ) [ 2 γ + \sqrt{{( 1 - γ )}^{2} - {( λ - 1 )}^{2} {η}^{2}} ] , & λ \leq {λ}_{c} \\ \frac{γ ( 1 + λ )}{1 + γ} , & λ > {λ}_{c}\end{matrix} \qquad ( 4 )$$

In some aspects, a limit of elongation λₐ is determined using the following Eq. 5:

$${λ}_{a} = 1 + \frac{k}{η} \sqrt{2 ( 1 - {γ}^{2} ) - {{k}^{2} ( 1 + γ )}^{2}} \qquad ( 5 )$$

wherein R_{U}−R_{L}=k²R_{U}.

In some aspects, an actual expansion ratio Rₐ is determined using the following Eq.6:

$${R}_{a} = \frac{1}{2} ( 1 + {λ}_{a} ) \qquad ( 6 )$$

In some aspects, the one or more processors are further configured to: initiate a cutting process with a device, wherein the device comprises an adaptive cutting mechanism having a multi-modal cutting head and a force-feedback sensor integrated into the cutting mechanism to monitor a resistance during cutting for real-time adjustments to a cutting depth and a cutting speed.

In some aspects, the one or more processors are further configured to: place a harvested graft in the device, wherein the device further comprises a microfluidic tissue support that uses micro-fluidics to maintain a specific position and a tension of the harvested graft during the cutting process of the parallel cutting pattern or the complex geometrical cutting pattern without damaging tissue.

In some aspects, the one or more processors are further configured to: expand the harvested graft and receiving from the integrated system a real-time feedback on achieved expansion ratio as the harvested graft is expanded.

In some aspects, the real-time feedback on the achieved expansion ratio further comprises providing an alert when the harvested graft approaches a selected expansion point, and wherein the selected expansion point is based on a predictive model and sensor data.

In some aspects, the one or more processors are further configured to: log data including parameters recorded by the integrated system of a skin grafting procedure for future reference and analysis.

In some aspects, the one or more processors are further configured to: conduct a post-procedure analysis wherein the integrated system provides a summary of a skin grafting procedure including an actual expansion ratio and deviations from predictions.

In some aspects, the complex geometric cutting pattern includes kirigami-inspired cuts.

In some aspects, the one or more processors are further configured to: adjust the device in real-time using the resistance monitored by the force-feedback sensor during the cutting process.

In some aspects, the adjustment of the device further comprises allowing the integrated system to adjust the cutting depth and the cutting speed in real-time.

In another aspect, a method for minimizing secondary contracture in split-thickness skin graft surgeries includes selecting, from an integrated system, a desired expansion ratio for a specific skin graft expansion in a patient; receiving, by the integrated system, an input of a set of geometric constraints; determining, by the integrated system, a first set of cutting patterns based on the desired expansion ratio and the input set of geometric constraints, wherein the first set of cutting patterns satisfies both the desired expansion ratio and the set of geometric constraints; and outputting, by the integrated system, the first set of cutting patterns, the first set of cutting patterns including a cutting pattern including one or more of a parallel cutting pattern or a complex geometrical cutting pattern.

In some aspects, the determining of the first set of cutting patterns includes selecting the first set of cutting patterns from a larger set of cutting patterns stored in the integrated system, the selecting of the first set of cutting patterns being based on which of the larger set of cutting patterns satisfies both the desired expansion ratio and the set of geometric constraints.

In some aspects, the larger set of cutting patterns are selected prior to the selecting of the desired expansion ratio. In some aspects, the larger set of cutting patterns are stored in a memory of the integrated system.

In some aspects, the larger set of cutting patterns are selected by excluding possible cutting patterns that: result in geometric overlap between adjacent cuts, exceed predefined limits on spacing between cuts, have fewer than a minimum number of cuts in a horizontal and a vertical direction, and are unable to achieve the desired expansion ratio.

In some aspects, the geometric overlap between adjacent cuts is determined using a cut length L, a horizontal spacing between cuts d, and a vertical gap between cut ends g.

In some aspects, the selecting of the desired expansion ratio R from the integrated system for the specific skin graft expansion in the patient comprises determining R using the following Eq. 2:

$$R = \frac{1}{2} ( \frac{1 + λ}{1 + γ} ) [ 2 γ + \sqrt{{( 1 - γ )}^{2} - {ϕ ( λ - 1 )}^{2} {η}^{2}} ] \qquad ( 2 )$$

wherein λ=1+(δ/d), γ=g/L, and η=d/L.

In some aspects, an upper limit for the extension ratio R_{U} is determined using the following Eq. 3:

$${R}_{U} = \frac{1}{2} ( 1 + λ ) \qquad ( 3 )$$

In some aspects, a lower limit for the extension ratio R_{L} is determined using the following Eq. 4:

$${R}_{L} = \{ \begin{matrix}\frac{1}{2} ( \frac{1 + λ}{1 + γ} ) [ 2 γ + \sqrt{{( 1 - γ )}^{2} - {( λ - 1 )}^{2} {η}^{2}} ] , & λ \leq {λ}_{c} \\ \frac{γ ( 1 + λ )}{1 + γ} , & λ > {λ}_{c}\end{matrix} \qquad ( 4 )$$

In some aspects, a limit of elongation λₐ is determined using the following Eq. 5:

$${λ}_{a} = 1 + \frac{k}{η} \sqrt{2 ( 1 - {γ}^{2} ) - {{k}^{2} ( 1 + γ )}^{2}} \qquad ( 5 )$$

wherein R_{U}−R_{L}=k²R_{U}.

In some aspects, an actual expansion ratio Rₐ is determined using the following Eq.6:

$${R}_{a} = \frac{1}{2} ( 1 + {λ}_{a} ) \qquad ( 6 )$$

In some aspects, the method further comprises initiating a cutting process with a device, wherein the device comprises an adaptive cutting mechanism having a multi-modal cutting head and a force-feedback sensor integrated into the cutting mechanism to monitor a resistance during cutting for real-time adjustments to a cutting depth and a cutting speed.

In some aspects, the method further comprises placing a harvested graft in the device, wherein the device further comprises a microfluidic tissue support that uses micro-fluidics to maintain a specific position and a tension of the harvested graft during the cutting process of the parallel cutting pattern or the complex geometrical cutting pattern without damaging tissue.

In some aspects, the method further comprises expanding the harvested graft and receiving from the integrated system a real-time feedback on achieved expansion ratio as the harvested graft is expanded.

In some aspects, the real-time feedback on the achieved expansion ratio further comprises providing an alert when the harvested graft approaches a selected expansion point, and wherein the selected expansion point is based on a predictive model and sensor data.

In some aspects, the method further comprises logging data including parameters recorded by the integrated system of a skin grafting procedure for future reference and analysis.

In some aspects, the method further comprises conducting a post-procedure analysis wherein the integrated system provides a summary of a skin grafting procedure including an actual expansion ratio and deviations from predictions.

In some aspects, the complex geometric cutting pattern includes kirigami-inspired cuts.

In some aspects, the method further comprises adjusting the device in real-time using the resistance monitored by the force-feedback sensor during the cutting process.

In some aspects, the adjustment of the device further comprises allowing the integrated system to adjust the cutting depth and the cutting speed in real-time.

In another aspect, the present disclosure relates to a non-transitory computer readable medium storing instructions for minimizing secondary contracture in split-thickness skin graft surgeries that, when executed by one or more processors, cause the one or more processors to: select, from an integrated system, a desired expansion ratio for a specific skin graft expansion in a patient; receive, by the integrated system, an input of a set of geometric constraints; determine, by the integrated system, a first set of cutting patterns based on the desired expansion ratio and the input set of geometric constraints, wherein the first set of cutting patterns satisfies both the desired expansion ratio and the set of geometric constraints; and output, by the integrated system, the first set of cutting patterns, the first set of cutting patterns including a cutting pattern including one or more of a parallel cutting pattern or a complex geometrical cutting pattern.

In another aspect, a system for minimizing secondary contracture in split-thickness skin graft surgeries includes a device; and one or more processors in communication with the device, the one or more processors configured to: select, from an integrated system, a desired expansion ratio for a specific skin graft expansion in a patient; receive, by the integrated system, an input of a set of geometric constraints; determine, by the integrated system, a first set of cutting patterns based on the desired expansion ratio and the input set of geometric constraints, wherein the first set of cutting patterns satisfies both the desired expansion ratio and the set of geometric constraints; and output, by the integrated system, the first set of cutting patterns, the first set of cutting patterns including a cutting pattern including one or more of a parallel cutting pattern or a complex geometrical cutting pattern.

In some aspects, the determining of the first set of cutting patterns includes selecting the first set of cutting patterns from a larger set of cutting patterns stored in the integrated system, the selecting of the first set of cutting patterns being based on which of the larger set of cutting patterns satisfies both the desired expansion ratio and the set of geometric constraints.

In some aspects, the larger set of cutting patterns are selected prior to the selecting of the desired expansion ratio. In some aspects, the larger set of cutting patterns are stored in a memory of the integrated system.

In some aspects, the larger set of cutting patterns are selected by excluding possible cutting patterns that: result in geometric overlap between adjacent cuts, exceed predefined limits on spacing between cuts, have fewer than a minimum number of cuts in a horizontal and a vertical direction, and are unable to achieve the desired expansion ratio.

In some aspects, the geometric overlap between adjacent cuts is determined using a cut length L, a horizontal spacing between cuts d, and a vertical gap between cut ends g.

In some aspects, the selecting of the desired expansion ratio R from the integrated system for the specific skin graft expansion in the patient comprises determining R using the following Eq. 2:

$$R = \frac{1}{2} ( \frac{1 + λ}{1 + γ} ) [ 2 γ + \sqrt{{( 1 - γ )}^{2} - {ϕ ( λ - 1 )}^{2} {η}^{2}} ] \qquad ( 2 )$$

wherein λ=1+(δ/d), γ=g/L, and η=d/L.

In some aspects, an upper limit for the extension ratio R_{U} is determined using the following Eq. 3:

$${R}_{U} = \frac{1}{2} ( 1 + λ ) \qquad ( 3 )$$

In some aspects, a lower limit for the extension ratio R_{L} is determined using the following Eq. 4

$${R}_{L} = \{ \begin{matrix}\frac{1}{2} ( \frac{1 + λ}{1 + γ} ) [ 2 γ + \sqrt{{( 1 - γ )}^{2} - {( λ - 1 )}^{2} {η}^{2}} ] , & λ \leq {λ}_{c} \\ \frac{γ ( 1 + λ )}{1 + γ} , & λ > {λ}_{c}\end{matrix} \qquad ( 4 )$$

In some aspects, a limit of elongation λₐ is determined using the following Eq. 5:

$${λ}_{a} = 1 + \frac{k}{η} \sqrt{2 ( 1 - {γ}^{2} ) - {{k}^{2} ( 1 + γ )}^{2}} \qquad ( 5 )$$

wherein R_{U}−R_{L}=k²R_{U}.

In some aspects, an actual expansion ratio Rₐ is determined using the following Eq.6:

$${R}_{a} = \frac{1}{2} ( 1 + {λ}_{a} ) \qquad ( 6 )$$

In some aspects, the one or more processors are further configured to: initiate a cutting process with a device, wherein the device comprises an adaptive cutting mechanism having a multi-modal cutting head and a force-feedback sensor integrated into the cutting mechanism to monitor a resistance during cutting for real-time adjustments to a cutting depth and a cutting speed.

In some aspects, the one or more processors are further configured to: place a harvested graft in the device, wherein the device further comprises a microfluidic tissue support that uses micro-fluidics to maintain a specific position and a tension of the harvested graft during the cutting process of the parallel cutting pattern or the complex geometrical cutting pattern without damaging tissue.

In some aspects, the one or more processors are further configured to: expand the harvested graft and receiving from the integrated system a real-time feedback on achieved expansion ratio as the harvested graft is expanded.

In some aspects, the real-time feedback on the achieved expansion ratio further comprises providing an alert when the harvested graft approaches a selected expansion point, and wherein the selected expansion point is based on a predictive model and sensor data.

In some aspects, the one or more processors are further configured to: log data including parameters recorded by the integrated system of a skin grafting procedure for future reference and analysis.

In some aspects, the one or more processors are further configured to: conduct a post-procedure analysis wherein the integrated system provides a summary of a skin grafting procedure including an actual expansion ratio and deviations from predictions.

In some aspects, the complex geometric cutting pattern includes kirigami-inspired cuts.

In some aspects, the one or more processors are further configured to: adjust the device in real-time using the resistance monitored by the force-feedback sensor during the cutting process.

In some aspects, the adjustment of the device further comprises allowing the integrated system to adjust the cutting depth and the cutting speed in real-time.

In another aspect, an integrated system for optimizing skin graft expansion in split-thickness skin graft surgeries comprises a physical device such as, but not limited to, a smart meshing tool with interchangeable cutting heads. Also included is an integrated software system that controls the device and provides predictive modeling. Sensors may be used for including force-feedback and elasticity sensor data. A predictive model incorporates mechanical principles and surgical intuition.

In another aspect, several components are integrated such as a meshing device that has a construct of a base unit with a precision-controlled cutting mechanism. The design may include interchangeable cutting heads for both conventional parallel cutting and kirigami-inspired patterns. The device may further include integrated motors and actuators for precise control of cutting depth and pattern. A sensor array may also be included, for example, such as an embedded force-feedback sensor(s) in the cutting mechanism to measure resistance during cutting. The sensors may also include incorporation of elasticity sensors to assess skin properties in real-time. A control system may also be included such as a microcontroller-based system to manage device operations. A user interface for surgeon's interaction and control may be included in the control system. Software components may also be included such as, but not limited to, predictive modeling of graft expansion, real-time adaptation of cutting patterns based on skin properties, and optimization of graft usage for maximum coverage. A database system may also be utilized for logging procedure parameters and outcomes. Depending on the specific implementation, all hardware components can be integrated and connected to a central control system that manages their operation. Such connections may ensure seamless communication between the physical device and software components.

In another aspect, the present integrated device and approach allow for a more precise, predictable, and optimized skin graft meshing process. In pre-surgery planning, for example, input patient data and desired outcomes may be entered into the software system. The system simulates various cutting patterns and predicts expansion ratios. Skin assessment may take place with the device on the donor site. Elasticity sensors may assess skin properties. The system may adjust its predictive model based on these measurements.

In yet another aspect, the split-thickness skin graft is harvested using conventional methods, but with the following modifications. In the meshing process, the following steps may be done. Placing a harvested graft in the device. Selecting a desired expansion ratio or coverage area. Receiving from the system recommendations for optimal cutting patterns (such as conventional or kirigami-inspired). Initiating the cutting process. Obtaining real-time adjustment with force-feedback sensors that monitor resistance during cutting. Allowing the system to adjust cutting depth and speed in real-time for optimal results. As the graft is expanded, the system provides real-time feedback on achieved expansion ratio. It alerts the surgeon when approaching the optimal expansion point, based on its predictive model and sensor data. Data logging is available where the system records all parameters of the procedure for future reference and analysis. A post-procedure analysis may be utilized where the software provides a summary of the procedure, including achieved expansion ratio and any deviations from predictions.

There are many advantages and distinguishing features about the present device and method. Some include the following. For example, predictable outcomes in both modes of skin grafting, such as both conventional and kirigami modes, deliver reliable results, catering to surgeon preference and offering improved control over expansion ratios. Again, there is reduced donor site trauma as both modes require fewer and shorter cuts compared to current state-of-the-art, thereby the present device and method minimize the amount of healthy skin harvested. The kirigami mode offers enhanced efficiency and control, enabling dual-directional expansion and precise tailoring of the mesh to meet specific wound requirements. This improves overall efficiency and minimizes wasted skin. Additionally, tension within the graft is minimized. In both modes, this allows for the prevention of the activation of cells caused by mechanical tension generated within skin grafts. The present disclosure introduces a novel approach to skin graft meshing by offering the flexibility to leverage both conventional and kirigami-inspired designs, while ensuring consistent and predictable outcomes in both modes.

In yet another aspect, the present device and method provide enhanced conventional parallel cutting. This mode caters to situations where the surgeon prefers a familiar approach. This will create the standard parallel cut patterns employed in existing meshing devices. However, the design incorporates advancements that lead to an improved expansion ratio compared to conventional devices. This is achieved with fewer and shorter cuts, minimizing donor site trauma. The surgeon may also use other geometric patterns such as, but not limited to, kirigami-inspired design with precise control. This mode harnesses the power of metamaterial analysis to generate intricate cut patterns inspired by kirigami. These patterns go beyond the limitations of conventional parallel cuts, offering significantly higher expansion ratios with fewer and shorter cuts. In both cases, whether using conventional parallel cutting or kirigami-inspired cuts, this disclosure reliably delivers the promised expansion ratio, unlike current meshing devices, while also minimizing tension within skin grafts. This reduction in tension helps prevent the activation of cells within the graft, thereby reducing the risk of postoperative complications.

In other aspects, the following structural features are made available by the present device and method. The combination of adaptive cutting mechanisms, integrated sensors, and real-time control systems, enable the kirigami-inspired design with precise control. The system allows for complex, customizable cutting patterns that can be dynamically adjusted based on the specific properties of each skin graft and the desired expansion outcomes. An adaptive cutting mechanism allows a dynamically adjustable cutting head that can switch between conventional parallel cuts and complex kirigami-inspired patterns. Micro-actuators or a multi-axis positioning system are used to achieve precise, programmable cutting paths. Depending on the implementation, integrated skin property sensors such as elasticity sensors embedded in the device's contact surface measure skin mechanical properties in real-time before and during the cutting process. A force-feedback cutting system incorporates precision force sensors integrated into the cutting mechanism. These monitor resistance during cutting, allowing for real-time adjustments to cutting depth and speed. Multi-modal cutting heads allow interchangeable cutting heads designed for different patterns, and may include a rotating drum with multiple cutting patterns or a reconfigurable cutting surface. A micro-fluidic tissue support provides for a tissue-holding mechanism that uses micro-fluidics to keep the graft in optimal position and tension during cutting. This allows for more complex cutting patterns without damaging the tissue. An adaptive tensioning system provides for an automated system that adjusts the tension of the graft during cutting based on real-time sensor data. This enables maintenance of optimal tissue conditions for complex kirigami-inspired cuts.

In yet another aspect, provided is a high-precision depth control that comprises a system for ultra-fine control of cutting depth, potentially using piezoelectric actuators. This allows for varying depth cuts within a single pattern, a key feature for kirigami-inspired designs. Also provided may be an integrated imaging system using a high-resolution imaging system that maps the graft in real-time. This feature allows guidance of the cutting process and allows for immediate verification of cut patterns. Another addition is a programmable pattern generator that comprises a software module that generates custom cutting patterns based on input parameters and sensor data. This enables the creation of patient-specific kirigami-inspired patterns optimized for each graft. A closed-loop control system provides continuous adjustments of cutting parameters based on real-time feedback from all sensors. This ensures precise control and adaptation throughout the cutting process.

The present innovative design addresses problems of the current state-of-the-art skin grafting by minimizing the mechanical tension generated within the skin graft. By reducing this tension, the approach decreases the likelihood of fibroblast activation, thereby lowering the risk of Secondary Contracture—a postoperative complication characterized by excessive scar tissue formation and contraction, which can restrict mobility and impair the function of the grafted area.

Chronic wounds—long-lasting injuries that the body cannot heal—can originate from burns, infection, skin cancer surgery, or ulcers associated with poor blood circulation, immobility, and diabetes. For patients requiring repair of widespread skin wounds, the expansion of harvested healthy skin to cover larger wounds is particularly important. This helps limit additional trauma to the patient by reducing the area of skin harvesting from the healthy part of the patient's body. However, achieving this requires accurate determination of the extent to which a harvested skin graft can be expanded to cover a larger wound area, the “meshing ratio” or “skin graft expansion ratio”. The graft expansion ratio as defined herein is the ratio of the area of the skin graft after expansion to the initial area of the harvested skin graft before expansion. Skin graft expansion ratios observed in clinical settings often deviate significantly from the nominal ratios claimed by manufacturers of current meshing devices used in skin graft surgeries. The need to develop a straightforward and predictive model, along with designing a meshing device based on this model, is further evident from recent studies illustrating that although surgeons widely recognize the tendency for actual ratios to fall short of nominal ones, they significantly overestimate these actual ratios by 55%, with no statistically significant difference between estimates by senior surgeons and residents.

The present innovation surpasses the current standard for skin graft meshing in many ways. First, it consistently achieves the claimed expansion ratio, setting it apart from conventional meshing devices that fail to achieve the claimed expansion ratio. Second, it enables higher expansion ratios while using fewer and shorter cuts. This reduces patient trauma by minimizing skin harvested from healthy donor sites and concurrently improves wound healing rates. Third, the design minimizes the tensile stress generated within skin grafts. As demonstrated in the present findings, static tensile stress can activate dermal fibroblasts, leading to their transition into a more contractile state. By reducing this stress, the present innovation prevents the activation of dermal fibroblasts, thereby mitigating the risk of postoperative complications such as “Secondary Contracture,” which is associated with the increased contractility of these cells. Fourth, while current devices employ conventional parallel cutting patterns that limit expansion to the direction perpendicular to the cuts, the present design also incorporates advanced kirigami-inspired patterns that allow for simultaneous expansion in both directions. This dual-directional expansion provides surgeons with greater flexibility to cover wounds of varying shapes more effectively, reducing the need for additional grafts and enhancing overall surgical outcomes that are further explained here in the detailed description and accompanying figures.

### BRIEF DESCRIPTION OF THE DRAWINGS

The patent or application file contains at least one drawing executed in color. Copies of the patent or patent application with color drawing(s) will be provided by the Office upon request and payment of the necessary fee.

To assist those of skill in the art in making and using the disclosed composition and method, reference is made to the accompanying figures, wherein:

FIGS. 1(A)-1(E) are schematics illustrating visual representations of an analytical model to predict skin graft expansion ratio based on the expansion mechanisms of a representative skin unit.

FIGS. 2(A)-2(B) are graphs illustrating a conventional model's failure to capture the mechanics of skin grafting as predicted by finite element method (FEM) analyses.

FIGS. 2(C)-2(E) are graphs illustrating variations with respect to γ=g/L that were better captured by proposed model of Eq. (2).

FIGS. 3(A)-3(C) are graphs illustrating an expansion ratio of skin grafts with low meshing ratios that increases consistently with skin extension ratio, while those with high meshing ratios may plateau due to significant vertical shrinkage.

FIG. 3(D) is a schematic illustrating a linear increase in an expansion ratio with A for the low meshing ratio Rₙ=1.5 at three different states of skin expansion (i) to (iii), where there is minimal vertical shrinkage as skin grafts expand horizontally.

FIG. 3(E) is a schematic illustrating, conversely to FIG. 3(D), that for higher meshing ratios, significant vertical shrinkage can occur at large extension ratios, offsetting horizontal expansion and maintaining overall graft area, as observed in panel FIG. 3(B)(examples I-III) and panel FIG. 3(C)(examples III-IV).

FIG. 4(A) is a graph illustrating four examples of meshing parameters (L,d,g) that yield the same meshing ratio Rₙ=6.

FIG. 4(B) is a schematic illustrating that below the lock-up point λc, rotation of the incision arms primarily drives graft elongation and expansion.

FIGS. 5(A)-5(B) are schematics illustrating examples of how a strain-stiffening property of skin affects the skin graft expansion ratio, while stiffness and Poisson's ratio of the skin do not.

FIGS. 6(A)-6(I) are graphs illustrating a new model that predicts experimental data and trends, while the conventional model does not.

FIGS. 7(A)-7(F) illustrate experimental observations and computational simulations of the effects of sustained stretch on tissue contractility.

FIGS. 8(A)-8(F) illustrate experimental observations and computational simulations of the effects of sustained stretch on passive responses of tissues.

FIGS. 9(A)-9(D) illustrate time and strain dependent ECM mechanics.

FIGS. 10(A)-10(I) illustrate stresses experienced by cells in a 3D ECM regulated by time and strain-dependent ECM mechanics.

FIGS. 11(A)-11(C) illustrate stretching of skin tissues for a long duration can induce mechanical memory in cells and cause their long-term activation in a non-monotonic manner with tissue strain magnitude.

FIG. 12 is a flowchart illustrating a method of meshed skin graft planning.

FIGS. 13(A)-13(F) are graphs illustrating feasible parameter configurations for expansion ratio 1.3 with a square initial graft geometry.

FIGS. 14(A)-14(F) are graphs illustrating feasible parameter configurations for expansion ratio 3 with a square initial graft geometry.

FIGS. 15(A)-15(F) are graphs illustrating feasible parameter configurations for expansion ratio 1.5 with rectangular (2:1 aspect ratio) initial graft geometry.

FIGS. 16(A)-16(F) are graphs illustrating feasible parameter configurations for expansion ratio 1.5 with a square initial graft geometry.

FIGS. 17(A)-17(F) are graphs illustrating feasible parameter configurations for expansion ratio 2 with a square initial graft geometry.

FIG. 18 is a block diagram of an exemplary computer system for implementing methods of the present disclosure.

### DETAILED DESCRIPTION

The present design, among other things, enables and combines a device with a process for optimizing skin graft expansion in split-thickness skin graft surgeries. The device component includes a smart meshing tool with interchangeable cutting heads, capable of both conventional parallel cutting patterns and advanced kirigami-inspired patterns. This integrates with software that guides cutting. The process aspect involves a novel method for predicting and achieving optimal graft expansion ratios, and may also include incorporating real-time skin property assessment, adaptive cutting patterns, and a predictive model that accounts for both mechanical principles and surgical intuition. The disclosed process and associated system, as well as the integrated device, either alone or in combination, improve the accuracy, efficiency, and outcomes of skin graft procedures.

The predictive model, which may be implemented on a stand-alone system (independent of the device), performs parameter sweeps over incision length, horizontal spacing, and vertical spacing to identify cutting patterns that are both geometrically feasible and surgically practical. The predictive model can identify multiple valid cutting patterns for each target expansion ratio, offering the surgeon the ability to trade-off between fewer long incisions and numerous short incisions.

The process of identifying cutting patterns using the predictive model allows surgeons to determine minimum donor skin requirements before harvesting, identify all feasible meshing configurations for a target expansion ratio, and select configurations based on explicit trade-offs between expansion efficiency and incision burden. Clinical case examples discussed below illustrate application for a major burn patient with limited donor sites, and an elderly patient with healing-impairing comorbidities.

Another utility of the present innovation is in improving the skin graft meshing process for treating chronic wounds arising from burns, infections, surgeries, or diabetic ulcers. The understanding of skin mechanics gained through this innovation is applied to develop meshing techniques for other soft tissue grafts used in various surgeries. The present innovation may also be utilized in development of biocompatible materials by understanding how skin expands under stress and how skin cells respond to this stress that can inform the design of biocompatible materials that replicate the mechanical properties of skin, enhancing their effectiveness in wound closure applications. These materials could offer alternatives to skin grafts in certain situations.

### Expansion Limits Of Meshed Split-Thickness Skin Grafts

Split-thickness skin grafts are widely used to treat chronic wounds. Procedure design requires surgeons to predict how much a patch of the patient's own skin expands when it is meshed with rows of slits and stretched over a larger wound area. Accurate prediction of graft expansion remains a challenge, with current models overestimating the actual expansion, leading to suboptimal outcomes. Inspired by the principles of mechanical metamaterials, developed was a model that distinguishes between the kinematic rearrangement of structural elements and their stretching, providing a more accurate prediction of skin graft expansion. The present model was validated against extensive data from skin graft surgeries, demonstrating vastly superior predictive capability compared to existing methods. This metamaterial-inspired approach enables informed decision-making for potentially improving healing outcomes.

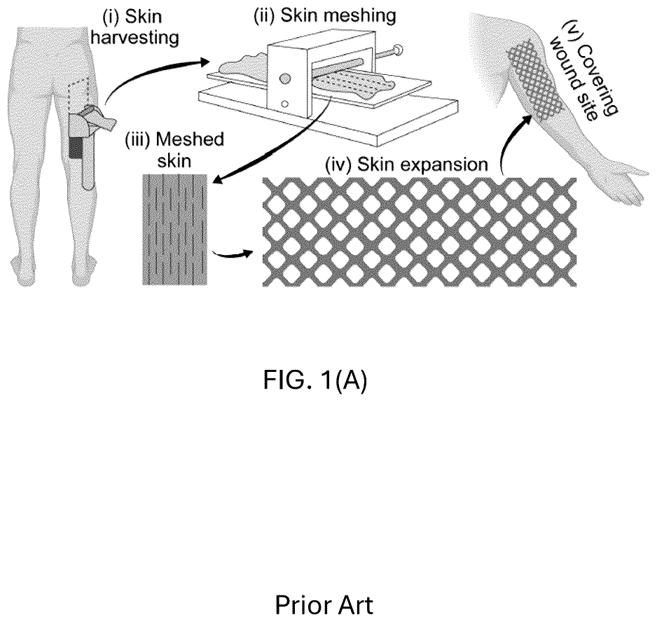

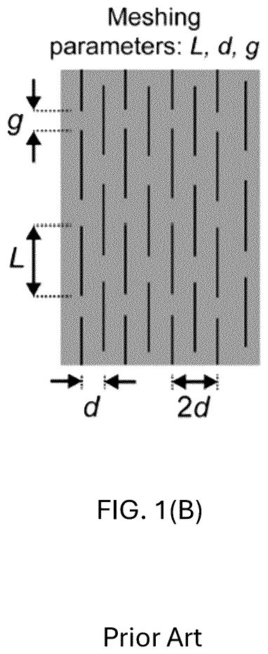

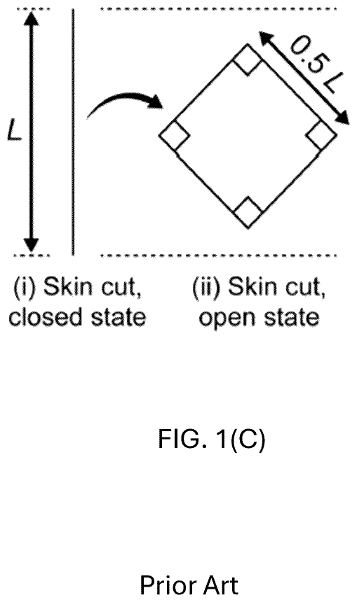

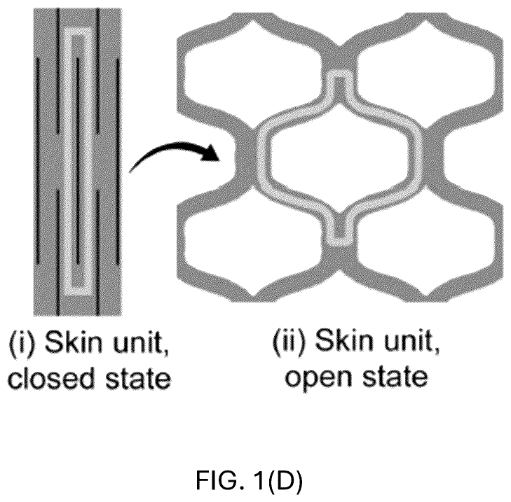

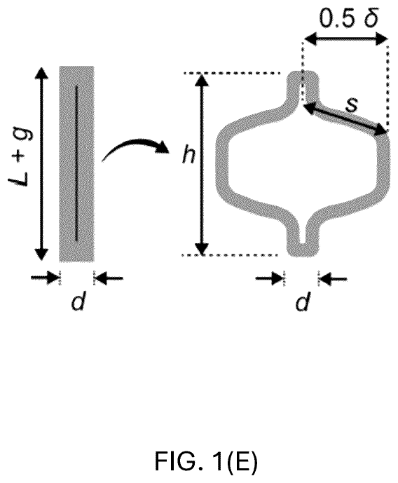

Chronic wounds—long-lasting injuries that the body cannot heal—can originate from burns, infection, skin cancer surgery, or ulcers associated with poor blood circulation, immobility, and diabetes. To cover chronic wounds and protect them from the environment and from pathogens, the wounds are often treated with split-thickness skin grafts. As shown in FIG. 1(A), these involve harvesting a thin layer of skin from a healthy part of the body (part (i) of FIG. 1(A)), meshing the harvested graft with a pattern with rows of slits (part (ii-iii) of FIG. 1(A)), expanding the graft (part (iv) of FIG. 1(A)), and subsequently transplanting the graft onto a wound site of a larger area (part (v) of FIG. 1(A)).

The meshing of split-thickness skin grafts both facilitates drainage of fluids from beneath the transplanted graft and enables the graft to be spread over a larger surface area. For patients requiring repair of widespread skin wounds, the expansion of harvested healthy skin to cover larger wounds is particularly important. This helps limit additional trauma to the patient by reducing the area of skin harvesting from the healthy part of the patient's body. However, achieving this requires accurate determination of the extent to which a harvested skin graft can be expanded to cover a larger wound area, the “meshing ratio” or “skin graft expansion ratio,” R. The graft expansion ratio is defined as the ratio of the area of the skin graft after expansion (part (iv) of FIG. 1(A)) to the initial area of the harvested skin graft before expansion (part (iii) of FIG. 1(A)). As shown in FIG. 1(B), the ratio depends on the meshing pattern cut into the skin graft, defined by three meshing parameters: the length of each cut (L), the spacing between cuts (d), and the length of the gaps between the ends of the cuts (g). Increasing the length of cuts (L) and reducing the distances between them (d and g) leads to greater expansion of the graft and higher expansion ratios.

As shown in FIG. 1(C), a conventional model to estimate the nominal expansion ratio, Rₙ, of skin grafts assumes that each slit transforms into a square when the graft is expanded. This assumption led the present investigators to the below equation (1) expressed as follows:

$${R}_{n} = 1 + [ \frac{1}{d} \times \frac{1}{L + g} \times {( \frac{L}{2} )}^{2} ] = 1 + \frac{1}{4 η ( 1 + γ )} \qquad ( 1 )$$

where γ=g/L and η=d/L, and a skin graft meshed with parameters L, d, and g is predicted to expand by Rₙ times its initial area. However, the actual expansion ratio (Rₐ) achieved after skin graft expansion can differ substantially from the nominal expansion ratio Rₙ, and from corrections to it associated with commercial skin grafting devices.

The need to develop a straightforward and applicable predictive model for actual expansion ratios is evident from the observation that despite surgeons widely recognizing the tendency for actual ratios to fall short of nominal ones, they significantly overestimated these actual ratios by 55%. This trend persists across surgeons with different levels of experience, as evidenced by survey data revealing no statistically significant difference between estimates provided by senior surgeons and residents. However, the mechanisms underlying these notable differences are uncertain, and there is currently no alternative model that can provide a straightforward and practical means to predict Ra. The mechanical mechanisms underlying skin graft expansion were studied using numerical simulations, and a theoretical model was built based upon these mechanisms. It was discovered that a nominal skin graft expansion ratio fails to predict data from finite element analyses.

To study the mechanical mechanisms underlying skin graft expansion and evaluate the predictive accuracy of the conventional model (Eq. (1)) for estimating skin graft expansion ratios, finite element simulations were performed to measure the expansion ratios of skin grafts meshed with different cutting patterns. To this end, a hyper-elastic constitutive model was first calibrated and used for modeling skin behavior by fitting it to uniaxial stretching experiments of unmeshed skin. Subsequently, the calibrated constitutive model was used to perform finite element simulations of meshed skin grafts, enabling present investigators to measure expansion ratio. Verification of the methodology is presented, and the code and a full description of the methodology are publicly available on GitHub (https://github.com/Farid-Alisafaei/Skin-Graft-Expansion-Ratio), incorporated by reference herein.

The skin expansion ratio in a simulations at a state in which each individual slit expands to a square under the assumptions of the conventional model in Eq. (1), was measured to compare the finite element simulation results with the conventional model in Eq. (1). In these simulations, a quarter of meshed skin grafts were modeled and considering for their symmetry. Then a horizontal displacement of D/2 (perpendicular to the direction of cuts) was applied at the right edge of meshed skin grafts. The dimensionless displacement D in the finite element method (FEM) simulations was determined by the equation D=(√2L/2)Nx, where Nx is the number of cuts in the horizontal direction for each case, L is the cut length, and √2L/2 represents the diameter of the perfect square (FIG. 1(C)).

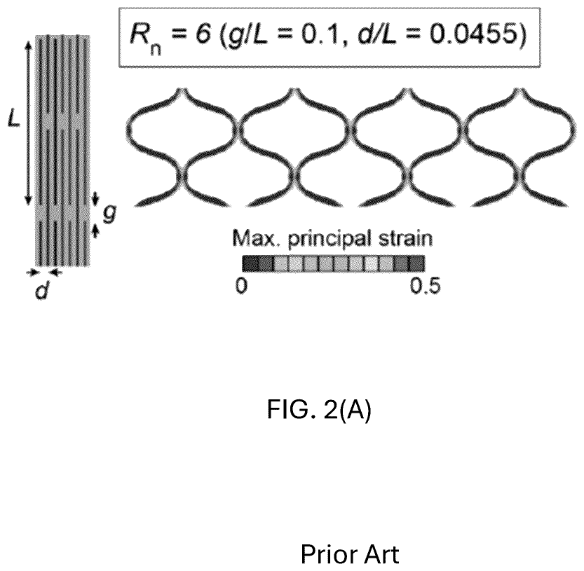

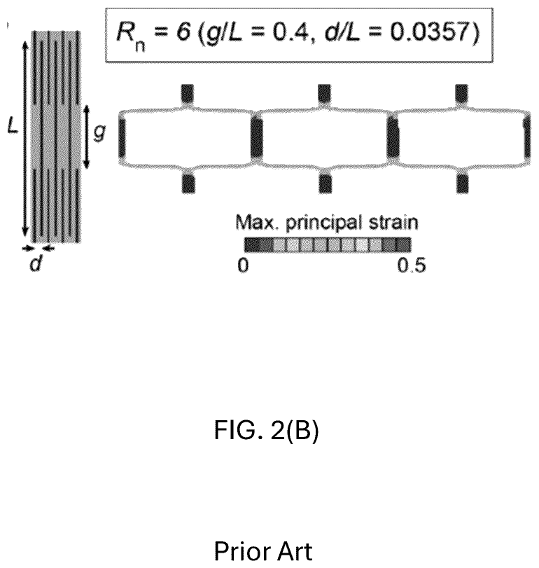

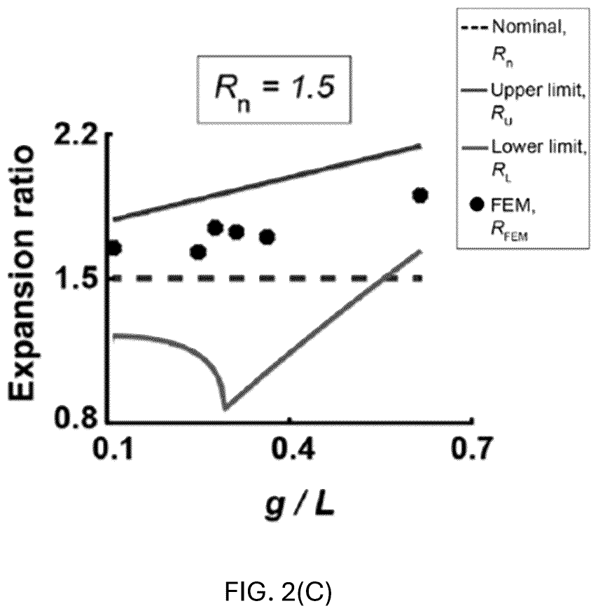

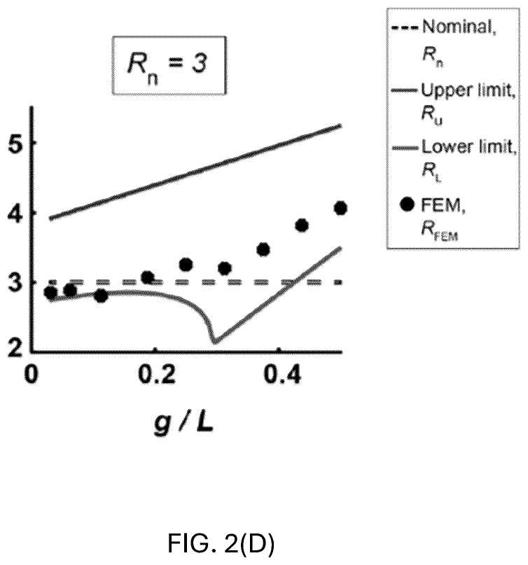

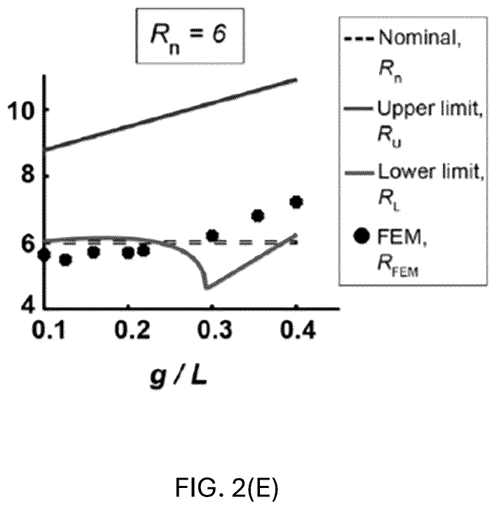

By comparing with the FEM simulations, a fundamental prediction of the conventional model in Eq. (1) was evaluated. The fundamental prediction is that all meshing patterns for which η(1+γ) is the same will yield the same expansion ratio. For example, the two meshing patterns in FIGS. 2(A)-(B) yield η(1+γ)=0.05 and thus should both have the same expansion ratio of Rₙ=6 because the parameters for panel A (γ=g/L=0.1, η=d/L=0.0455) and panel B (γ=g/L=0.4, η=d/L=0.0357) both yield this same expansion ratio when inserted into Eq. (1). The prediction of the conventional model for meshing ratios of Rₙ=1.5, 3, and 6, which are used clinically.

FIGS. 2(A)-2(B) illustrate sections of a skin graft meshed with two distinct sets of meshing parameters (L,d,g), sharing the same meshing ratio Rₙ=6 as determined from Eq. (1), depicted before and after expansion to a degree that, according to the conventional model, should open the slits into squares tilted at 45°. However, FEM simulations predicted different opening states for these two cases and showed significant strain generated in the skin. FEM simulations of skin grafts meshed with different sets of meshing parameters (L,d,g) that all yield the same expansion ratio Rₙ from the conventional model (Rₙ=1.5 FIG. 2(C), 3 FIG. 2(D), or 6 FIG. 2(E)). The prediction of the conventional model that all such grafts should have the same expansion ratio (dashed lines) was not borne out by simulations.

The conventional model (black dashed lines in FIGS. 2(C)-(E) did not predict finite element results (black solid circles in FIGS. 2(C)-(E). Finite element results predicted that expansion ratios for skin grafts with the same Rₙ from Eq. (1) but different sets of parameters (L, d, g) were not identical for all three of the clinically relevant values of Rₙ studied. Simulations also demonstrated that the two primary assumptions of the conventional model are not always accurate; the conventional model predicts that (i) each cut opens into a square, and (ii) the transition of the skin graft to this state occurs without stretching of the skin. However, the actual cut shapes can remarkably differ from square, and strains in the skin could be high. This was evident in strain and displacement patterns seen in the two examples shown in FIGS. 2(A)-(B), where the grafts had been stretched to a degree that should, according to the conventional model, yield square-shaped openings of the slits (displacement of D/2=(√2 L/4)Nx, as described earlier).

### Proposed Model

The proposed model captures the scaling of graft expansion with respect to skin grafting parameters and strain. As the conventional model failed to accurately predict graft expansion ratios, a mechanistic model was developed to elucidate the mechanisms of graft expansion and consequently improve the prediction of expansion ratios. The model captures the scaling of the expansion ratio with respect to the meshing parameters. The model predicts the expansion ratio for all degrees of opening, not just for one idealized degree of opening as in the conventional model, because skin grafts are not always expanded to their ultimate limit for placement on a wound site. The model is a simple equation, similar to the conventional model, enabling straightforward application in skin graft surgeries.

The expansion ratio of a meshed skin graft can be estimated by the expansion ratio of a representative skin unit, shown as the gray rectangle in part (i) of FIG. 1(D). As shown in FIG. 1(D), the proposed model that tracks the deformation of a representative skin unit and incorporates both an incision and the surrounding skin (gray rectangle). Within the meshed skin graft, there are N of these representative skin units, where N denotes the total number of cuts in the graft. These representative skin units consist of an incision surrounded by skin, with a width of d and a height of L+g in the closed state (FIG. 1E), yielding the area d(L+g) in the closed state. To calculate the area of the representative skin unit during graft expansion, the findings from finite element simulations were leveraged. These simulations demonstrated that as the skin graft transitions from a closed state to an open state, the representative skin unit changes from a rectangle (part (i) of FIG. 1(D)) to nearly a hexagon (part (ii) of FIG. 1(D). Based on this observation, the expanded area of the representative skin unit was approximated using the area of a hexagon (shown in FIG. 1(E)) as a function of δ, where δ represents the opening of the representative skin unit.

As shown in FIG. 1(E), the expansion ratio of the representative skin unit is calculated as the ratio of the expanded area to the initial area d(L+g). The area of the hexagon in FIG. 1(E) was calculated under two scenarios, representing different limits for the vertical contraction of skin grafts. In a first scenario, h is held constant, representing the upper bound of the expansion ratio dominated by skin elasticity. In the first scenario, the height of the skin unit (h) during skin graft expansion remains constant at the value in the closed state, h=L+g. Thus s, the length of the lateral arm in FIG. 1(E), increased as the skin graft expanded. This represents an upper limit R_{U} on the expansion ratio of skin grafts.

In a second scenario, s is held constant, representing the lower bound in which the skin does not stretch. In the second scenario, s remained constant as the incision opened during skin graft expansion, so that no stretching occurred as the lateral arms rotated into the direction of stretch. Consequently, h decreased and the skin graft shrank in the vertical direction with increasing δ. This represents a lower limit on the expansion ratio, R_{L}. The expansion ratio for each of these two cases was determined as a function of δ, using below equation (2):

$$R = \frac{1}{2} ( \frac{1 + λ}{1 + γ} ) [ 2 γ + \sqrt{{( 1 - γ )}^{2} - {ϕ ( λ - 1 )}^{2} {η}^{2}} ] \qquad ( 2 )$$

where the parameter φ=0 represents the upper limit of the expansion ratio (R_{U}), while 0=1 represents the lower limit (R_{L}), the extension ratio λ=1+(δ/d) represents the ratio of the new length to the original length of the graft in the horizontal direction (perpendicular to the orientation of cuts), γ=g/L, and η=d/L. Setting the parameter φ=0 yields the upper bound for the expansion ratio (corresponding to expansion from both rotation and stretching of incision arms), while φ=1 yields the lower bound (rotation only). An expansion ratio Rₐ occurs at the extension λₐ where these bounds diverge, the point at which surgeons encounter substantial tissue resistance and cease expansion

The upper limit for R is the case where φ=0, so that the upper limit is:

$${R}_{U} = \frac{1}{2} ( 1 + λ ) \qquad ( 3 )$$

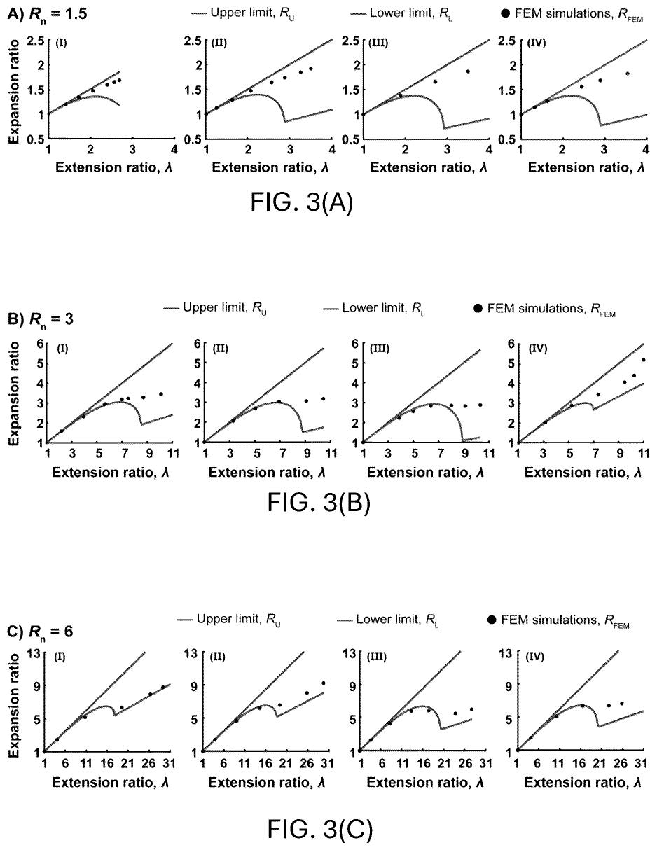

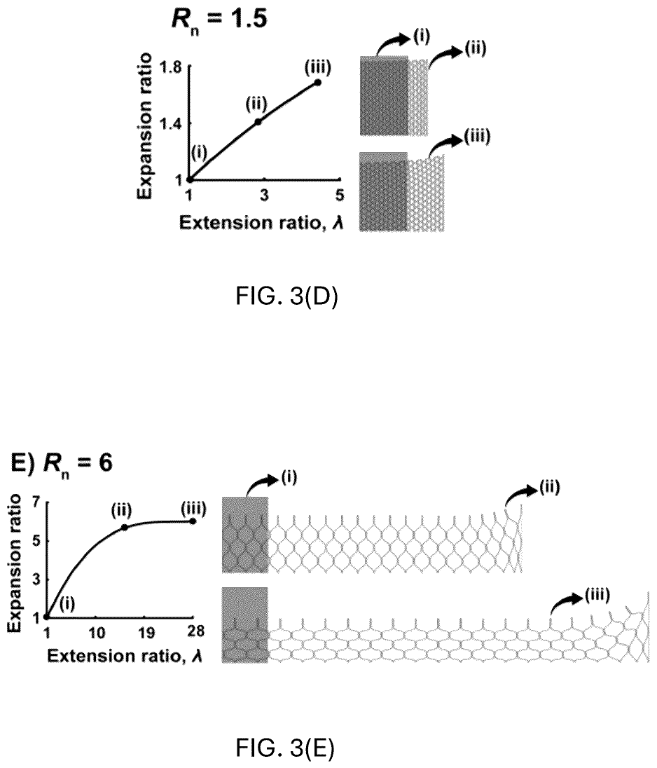

indicating that the upper limit increases linearly with λ, as illustrated later in FIGS. 3(A)-(C).

The lower bound case where φ=1 is applicable only for λ≤λc, where λc=(L+d−g)/d=1+[(1−γ)/η] is the lock-up point (critical extension ratio) beyond which the term under the radical in Eq. (2) is imaginary. At the lock-up point, the rotation of incision arms reaches its maximum value and subsequently beyond that point, any further extension of skin grafts must arise from elastic stretching of the skin. Approximating that extension, as arising only from the tissue outside of the gap regions in the graft, yields the first of the terms in the square brackets in Eq. (2), thus the lower bound is derived from equation (4) as shown here:

$${R}_{L} = \{ \begin{matrix}\frac{1}{2} ( \frac{1 + λ}{1 + γ} ) [ 2 γ + \sqrt{{( 1 - γ )}^{2} - {( λ - 1 )}^{2} {η}^{2}} ] , & λ \leq {λ}_{c} \\ \frac{γ ( 1 + λ )}{1 + γ} , & λ > {λ}_{c}\end{matrix} \qquad ( 4 )$$

Taken together, the expression for the skin graft expansion ratio R, presented in Eq. (2), takes the three meshing parameters (L, d, g) as input, similar to the conventional model in Eq. (1). As output, it determines both the upper R_{U} and lower R_{L} limits of the expansion ratio at any opening stage λ=1+δ/d.

FIGS. 2(C)-2(E) illustrate variations with respect to γ=g/L that were better captured by proposed model of Eq. (2). For low Rₙ, FEM results mirrored the upper bound, while for high Rₙ they mirrored the lower bound. In contrast to the conventional model (Eq. (1)), the limits associated with the proposed model, determined at the same opening displacement (δ=√2L/2 per unit cell), captured the generally increasing trends in expansion ratio with increasing g/L (FIGS. 2(C)-(E)). For low meshing ratios (Rₙ=1.5), the FEM results followed the upper limit curve (FIG. 2(C)), while for large meshing ratios (Rₙ=6), the FEM results followed the lower limit curve (FIG. 2(E)). For intermediate meshing ratios (Rₙ=3), FEM results fell between the upper and lower limit curves (FIG. 2(D)).

When a skin graft's expansion ratio can fall between the two limits during its transition from a closed state to open states and beyond. A relationship between the expansion R and extension λ of a skin graft is characterized through finite element simulations performed for a range of meshing patterns and nominal expansion ratios Rₙ (FIGS. 3(A)-(C)), and by comparing results to the limits. As shown in FIGS. 3(A)-(C), an expansion ratio of skin grafts with low meshing ratios increases consistently with the skin extension ratio, while those with high meshing ratios may plateau due to significant vertical shrinkage. FIGS. 3(A)-(C) show four examples (examples I-IV) that yield the same meshing ratio Rₙ determined by Eq. (1), covering meshing ratios of 1.5, 3, and 6. The expansion ratio was plotted against the extension ratio λ, where λ is defined as the width of the graft (in the direction of expansion) after expansion divided by the width of the graft before expansion. For the low meshing ratio Rₙ=1.5, the skin expansion ratio obtained from FEM simulations closely followed the upper limit curve derived from Eq. (2), increasing linearly with the skin extension ratio λ. However, for higher meshing ratios of 3 and 6, the FEM simulations approached the lower limit curve at high A and deviated from the initial linear relationship between expansion ratio and extension ratio.

As with the simulations performed at the nominal graft extension ratio λₙ in FIGS. 2(C)-(E), grafts with a low nominal meshing ratio (Rₙ=1.5) expanded in a way that followed the upper limit (FIG. 3(A)), increasing almost linearly with λ. Consistent with this, examination of the deformed meshes revealed that skin grafts meshed with low meshing ratios expanded horizontally without significant vertical shrinkage (FIG. 3(D)). As shown in FIG. 3(D) the linear increase in expansion ratio with λ for the low meshing ratio Rₙ=1.5 at three different states of skin expansion (parts (i) to (iii)), where there is minimal vertical shrinkage as skin grafts expand horizontally.

Also analogous to the results in FIGS. 2(C)-(E), grafts with a high meshing ratio (Rₙ=6) followed the lower limit curve (FIG. 3(C)). Unlike the upper limit curve, the lower limit curve demonstrated a linear increase with A only for small extension values λ. For larger extension, the slope of the lower limit curve gradually decreased with increasing λ, eventually leading to no further increase in the expansion ratio with increasing λ. Examination of deformed meshes revealed the source of this behavior: at high values of λ, skin grafts with high meshing ratios underwent significant vertical shrinkage that offset area gains due to horizontal expansion (FIG. 3(E)). As shown in FIG. 3(E), for higher meshing ratios, significant vertical shrinkage can occur at large extension ratios, offsetting horizontal expansion and maintaining overall graft area, as observed in examples I-III of FIG. 3(B) and examples III-IV of FIG. 3(C).

### Skin Graft Mechanics

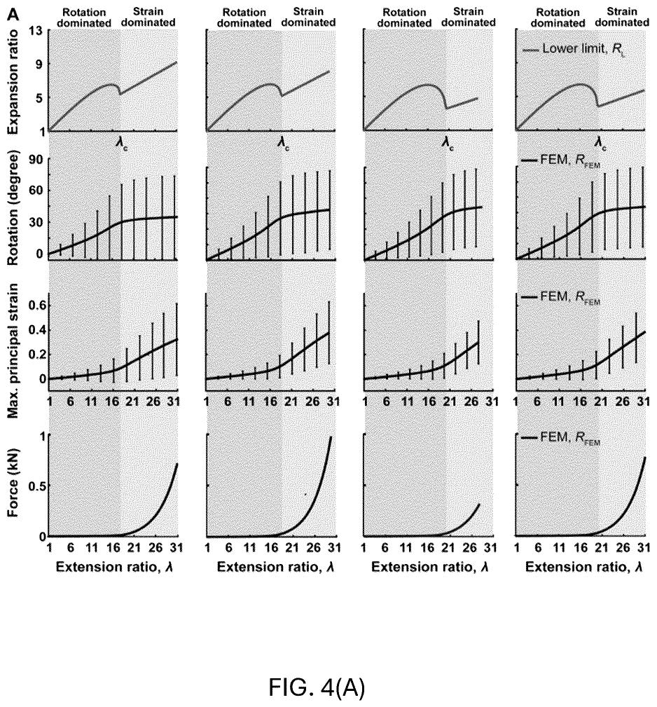

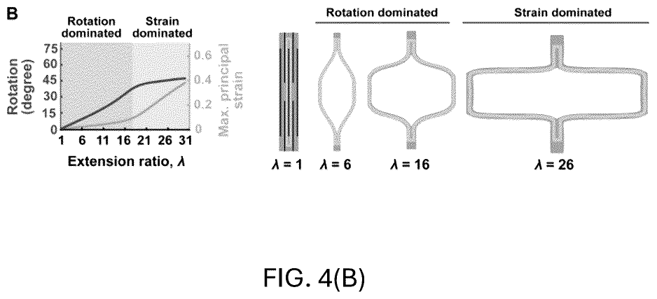

Skin graft mechanics are dominated by a rotation mechanism below the critical extension, and by stretch above it. The critical extension λc manifests as a local minimum in the lower limit expansion ratio (R_{L}) curve (FIG. 4(A)), and as described earlier, at this point the term under the radical in Eq. (2) becomes zero. To illustrate the mechanisms underlying this, plotted was the average rotation and average tensile strain in each finite element across the entire skin graft in the FEM simulations for skin grafts meshed with the nominal meshing ratio of Rₙ=6 (FIG. 4(A)). As shown in FIG. 4(A), at critical extension, rotation of incision arms reaches its maximum. Beyond this point, further elongation of skin grafts is solely based on skin straining. FIG. 4(A) shows four examples of meshing parameters (L,d,g) that yield the same meshing ratio Rₙ=6. For each example, the lower limit of the expansion ratio (R_{L}) from Eq. (2), along with the average rotation of elements, the tensile strain generated in elements, and the total force required for graft extension, all obtained from FEM simulation (with error bars indicating the maximum and minimum values observed in the FEM simulations) were plotted. The changes in the expansion ratio with those in rotation, strain, and force were compared as the incisions transitioned from a fully closed state (λ=1) to open states and beyond. The comparison revealed that at the extension ratio λ where the expansion ratio reaches a local minimum, denoted as the critical extension λc, the slope of rotation, strain, and force undergoes a drastic change. Beyond this point, there is a minimal increase in rotation, while strain and force experience rapidly increases.

For λ<λc, elongation of skin grafts was primarily associated with rotation of incision arms, with relatively low tensile strain (FIGS. 4(A)-(B)). Rotation increased to a plateau near λ=λc, beyond which further graft elongation was associated with a sharp increase in tensile strain within the graft (FIGS. 4(A)-(B)), as well as a sharp increase in the force (FIG. 4(A)) necessary to elongate it. FIG. 4 (B) indicates that below the critical extension λc, rotation of the incision arms primarily drives graft elongation and expansion. However, at the critical extension λc, the incision arms reach their maximum rotation, and further elongation of the skin graft beyond this point can only occur through stretching the skin.

### Strain-Stiffening of Collagen Affects the Expansion Ratio of Skin Grafts

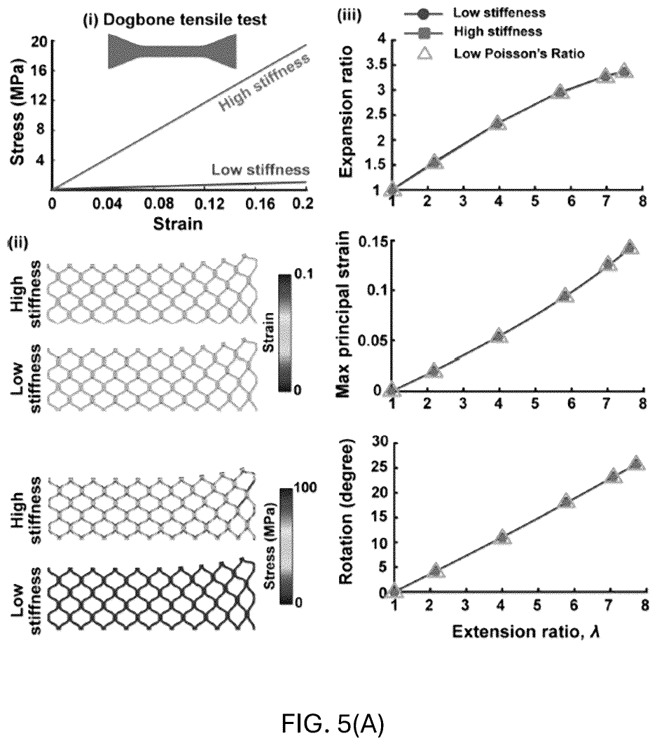

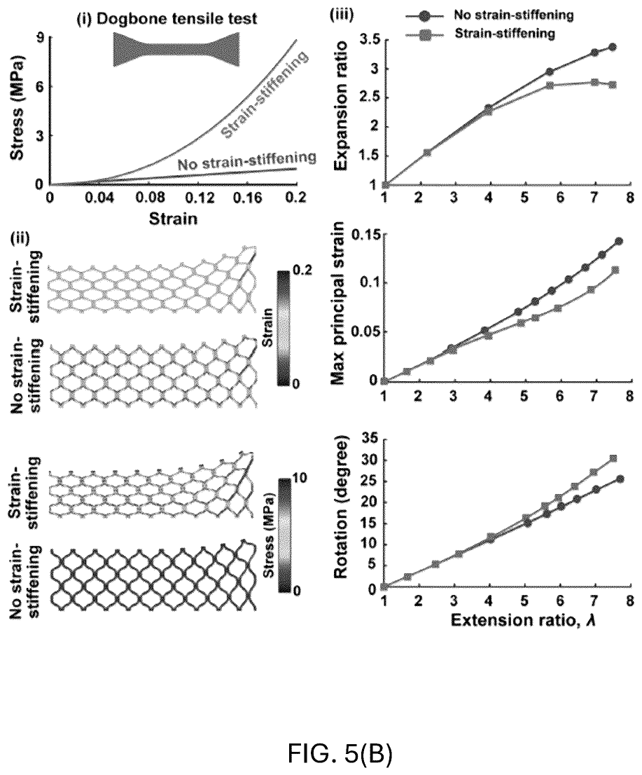

FIG. 5(A) illustrates a modeled skin as a hyper-elastic material with no strain-stiffening, using the neo-Hookean model, which requires only two material parameters: elastic modulus and Poisson's ratio. As shown in part (i) of FIG. 5(A), to assess the effect of elastic modulus, FEM simulations were performed by stretching unmeshed skin using two different elastic modulus values while maintaining a constant Poisson's ratio. Stress-strain curves derived from FEM simulations of dog-bone tensile tests on unmeshed skin illustrated these variations, with each curve's slope representing the skin's elastic modulus. As shown in part (ii) of FIG. 5(A), subsequently, the expansion of a meshed skin graft with Rₙ=3 were simulated using these two elastic modulus values. As shown in part (iii) of FIG. 5(A), measurements of the expansion ratio, average tensile strain, and average rotation showed identical outcomes, indicating that changes in elastic modulus did not affect the behavior of the meshed graft. Similarly, changing the skin's Poisson's ratio from 0.4 to 0.1 yielded no observable impact on the expansion ratio, average tensile strain, and average rotation.

Strain-stiffening of collagen affects the expansion ratio of skin grafts, but stiffness and Poisson's ratio do not. To investigate the effect of skin graft stiffness, the hyper-elastic neo-Hookean material model, commonly used in modeling biological tissues, was applied. Its two independent material parameters, Young's modulus and Poisson's ratio, were varied. Finite element simulations of a meshed skin graft with Rn=3 were performed using two different values of Young's modulus (E=5 and 100 MPa, cf. part (i) of FIG. 5(A)), but the same value of Poisson's ratio ν=0.4 (part (ii) of FIG. 5(A)). As expected, stresses were higher for the higher Young's modulus, but the expansion ratio, average level of tensile strain, and average level of element rotation were unchanged, indicating that changes to Young's modulus did not influence the behavior of the meshed skin graft (part (iii) of FIG. 5(A)). Altering Poisson's ratio of the skin (ν=0.1 and 0.4) affected neither these factors nor the stresses in the stretched skin grafts (part (iii) of FIG. 5(A)).

FIG. 5(B) illustrates integrating a strain-stiffening component into the hyper-elastic neo-Hookean model. As shown in part (i) of FIG. 5(B), this strain-stiffening behavior was apparent in the stress-strain curve derived from FEM simulations of dog-bone tensile tests on unmeshed skin, where unlike the neo-Hookean model, the slope of the stress-strain curve consistently increased with strain. Subsequently, the strain-stiffening model was used to simulate the extension of the meshed skin graft. Results revealed that skin strain-stiffening substantially diminished the expansion ratio of skin grafts at large extension ratios (A), primarily due to marked shrinkage occurring in the vertical direction.

To investigate the effect of skin's strain-stiffening, in which skin becomes more resistant to deformation as it is stretched, a strain-stiffening component was incorporated into the neo-Hookean model, resulting in an increasing slope of the stress-strain curve with tensile strain (part (i) of FIG. 5(B)). FEM simulations of the expansion of meshed skin grafts showed that strain-stiffening reduced expansion ratios (part (iii) of FIG. 5(B)), particularly at large values of λ. This reduction in the graft expansion ratio was due to increased shrinkage in the vertical direction as skin grafts expand horizontally, compared to the shrinkage that would occur without strain-stiffening (part (ii) of FIG. 5(B)). Taken together, the FEM simulations revealed that increasing the skin's strain-stiffening resulted in a decrease in the expansion ratio of skin grafts, approaching the lower limit of the expansion ratio (R_{L}).

### Estimates of Graft Expansion Ratio in Skin Graft Surgeries

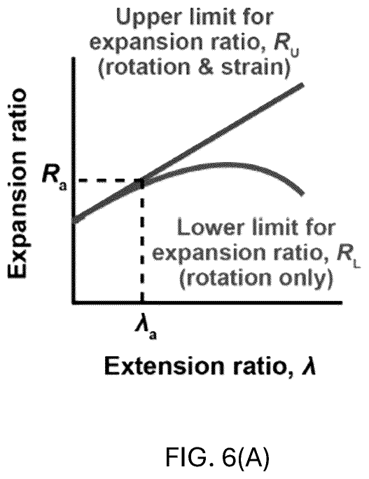

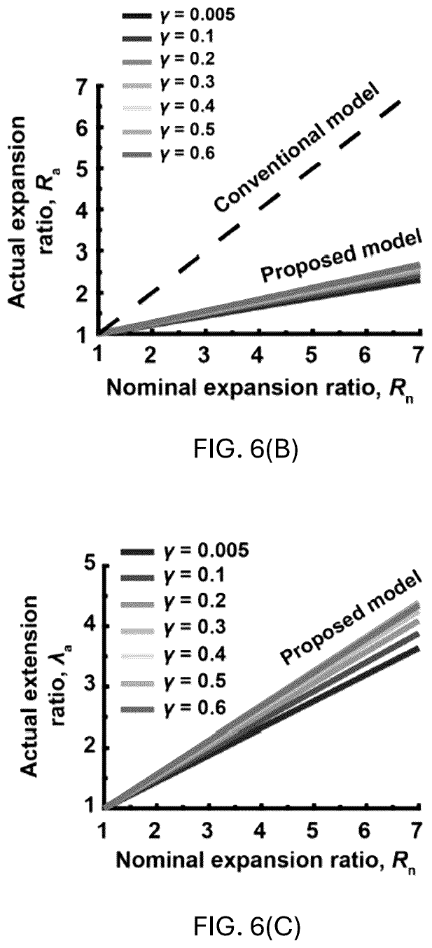

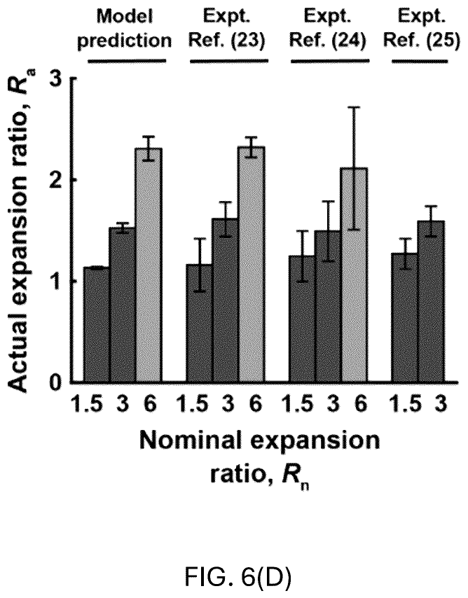

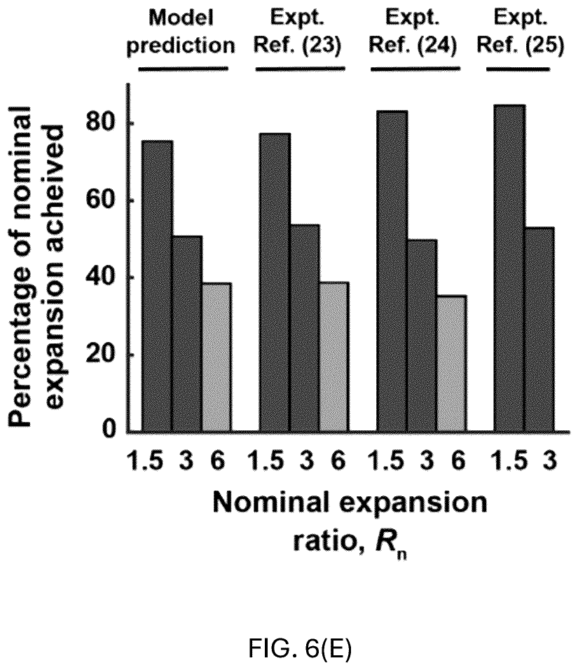

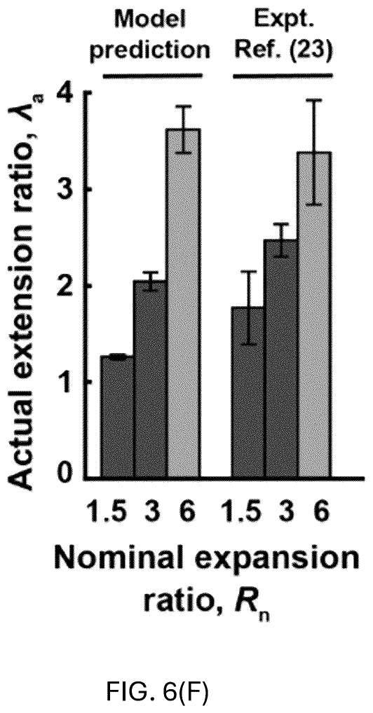

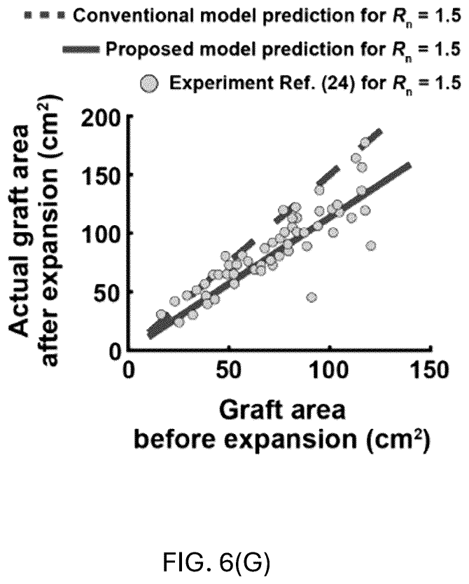

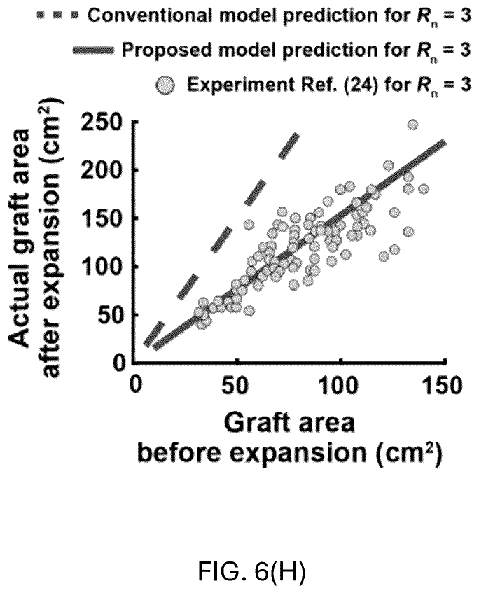

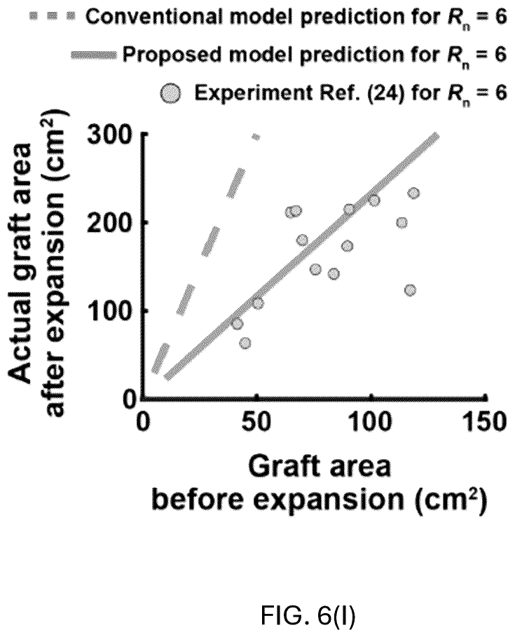

FIG. 6(A) illustrates that an example using (Eq. (2)) provides estimates of the upper and lower limits of the expansion ratio as a function of the graft extension ratio. The upper limit models graft expansion from both rotation and stretching of incision arms, while the lower limit models rotation without stretching of the incision arms. The limits diverge at λ=λa, where strain generated within the graft becomes significant and substantially increased force is required for further expansion. The bounds above for the graft expansion ratio represent limits for the cases of elasticity and structural rearrangement being separate, and for the entire graft undergoing only one of the two. However, real grafts undergo both behaviors to varying amounts as they expand. Thus, to convert these bounds into an estimate for the graft expansion ratio, the central questions are, first, what extension ratio (λ) is chosen by the surgeon in skin grafting, given that the graft expansion ratio R varies with λ, and second, the expansion ratio associated with this extension ratio. Because the upper limit R_{U} represents graft expansion resulting from both rotation and strain, while R_{L} represents expansion stemming only from rotation, the extension λ=λₐ at which these two limits diverge represents a threshold (FIG. 6(A)). Below λₐ, strain within the graft is negligible; beyond λₐ, substantial stretching of skin, and subsequently external forces, are needed to further expand the graft. Thus, λₐ represents a limit of elongation or stretching resistance that a surgeon is willing to accept. Defined herein is a ratio, k², so that R_{U}−R_{L}=k²R_{U} represents this threshold as a deviation between the upper and lower limits.

Fitting of data indicated that this threshold is typically just above 0.5%, so that k²=0.006. In some examples, k² ranges from 0.004 to 0.008. Expansion of the graft is stopped when upper and lower limits diverge near the k² value of approximately 0.006. Solving R_{U}−R_{L}=k²R_{U} for δ, gives an expression for δₐ, from which the actual extension ratio λₐ can be calculated as λa=1+(δₐ/d):

$${λ}_{a} = 1 + \frac{k}{η} \sqrt{2 ( 1 - {γ}^{2} ) - {{k}^{2} ( 1 + γ )}^{2}} \qquad ( 5 )$$

for γ≤(2−k²)/(2+k²). Substituting λₐ into R_{U} in Eq. (3), yields the following expression which determines the actual expansion ratio Rₐ of skin grafts simply as a function of the three grafting parameters (L, d, g), at the degree of extension λₐ as illustrated in Equation (6) below:

$${R}_{a} = \frac{1}{2} ( 1 + {λ}_{a} ) \qquad ( 6 )$$

For a range of nominal meshing ratios Rₙ spanning from 1.5 to 7 computed was Rₐ and λₐ (Eqs. (5) and (6)) for feasible combinations of the meshing parameters (L, d, g) that yield the same nominal expansion ratio Rₙ from Eq. (1). These were plotted in FIGS. 6(B)-(C), in which the bars illustrate the mean and standard deviation of feasible combinations for each nominal expansion ratio. FIGS. 6(B)-(C) illustrate multiple optimal extension ratios (λₐ) and associated expansion ratios (Rₐ, achieved at λₐ) were possible for a single value of Rₙ. Predictions for the expansion ratio Rₐ at the kinematic limit λₐ scaled almost linearly with Rₙ, but the slope varied dramatically with γ=g/L (FIG. 6(B)). Estimates of Rₐ were substantially lower than Rₙ because for all Rₙ, the upper and lower limit curves R_{U}(λ;L,g,d) and R_{L}(λ;L,g,d) diverge from each other long before reaching the nominal expansion ratio λₙ.

FIGS. 6(D)-(F) illustrate model predictions that were validated against experimental data for the actual expansion ratio Rₐ, and the actual extension ratio λₐ, as well as for the percentage of the nominal expansion achieved, calculated as 100(Rₐ/Rₙ). To validate model predictions, the model was compared with experimentally measured values of Rₐ reported in the literature, the latter consisting of measured expansion ratios in skin grafts meshed with nominal meshing ratios Rₙ of 1.5, 3, or 6. These experimental measurements demonstrated excellent agreement with the model predictions for Rₐ (FIG. 6(D)).

Another prediction of the model is that skin grafts with higher nominal meshing ratios achieve a lower percentage of the nominal expansion ratio. For example, when meshed with a nominal ratio of 6, the actual expansion ratio (Rₐ=2.3) achieved only 38% of the nominal expansion (Rₙ=6). This percentage was substantially lower compared to skin grafts meshed with a nominal ratio of 1.5, where the actual expansion ratio (Rₐ=1.13) achieved 75% of the nominal expansion, remarkably higher than 38% (FIG. 6(B)). The model suggests that this phenomenon arises because in cases of high Rₙ (e.g., 6), skin expansion follows the lower limit curve R_{L} (cf. FIGS. 2(E), 3(C)), so that divergence between the lower and upper limits occurs earlier. In contrast, for low Rₙ (e.g., 1.5), skin expansion follows the upper limit curve R_{U} (FIGS. 2(C), 3(A)) so that the deviation between the limits occurs later. These predictions were in excellent agreement with experimental data (FIG. 6(E)) showing that the actual-to-nominal expansion ratio (Rₐ/Rₙ) decreased with increasing Rₙ.

The model allows prediction of the extension ratio, λₐ, at which a surgeon would be expected to stop expanding a graft due to a substantial increase in the required force. Using Eq. (5), calculated was the extension ratio λₐ for different Rₙ values. Model predictions matched experimental measurements from the literature (FIG. 6(F)).

FIGS. 6(G)-(I) illustrate model predictions that were also validated across different donor sizes, comparing the actual graft area after expansion (Aₐ) to the graft area before expansion (A₀) for skin grafts with meshing ratios of 1.5, 3, and 6. For each meshing ratio, the model prediction Aₐ=Rₐ A₀ was compared to the conventional model prediction Aₐ=Rₙ A₀(Rₙ determined by Eq. (1)). To validate the model predictions across different donor sizes, the model's predictions for the Rₐ from experimentally measured values for skin grafts with varying initial areas (before expansion) were compared. Plotting the actual graft area after expansion (Aₐ) against the graft area before expansion (A₀) (FIGS. 6(G)-(I)) revealed that for meshing ratios of 1.5, 3, and 6, the proposed model predicted experimental data (Aₐ=Rₐ A₀), while the conventional model did not. Results confirmed the robustness of the model across varying donor sizes and meshing ratios. The model's accurate prediction of the actual expansion ratio underscores its potential for guiding skin grafting procedures.

### Method and Associated System for Optimizing Skin Graft Expansion

In one aspect, a method for optimizing skin graft expansion in split-thickness skin graft surgeries includes quantitative and patient-specific graft design decisions prior to harvesting. The method includes evaluating wound site dimensions and available donor skin, defining limits on a cutting pattern based on patient specific factors, determining feasible cutting patterns based on the defined cutting pattern limits, evaluated wound site dimensions, and available donor skin, and selecting a cutting pattern.

The evaluating of wound site dimensions and available donor skin includes determination by a surgeon of a required actual expansion ratio for complete wound coverage. This value represents the true expansion that will be achieved, not the nominal ratio from conventional models, which consistently overestimates actual performance.

The defining of limits on a cutting pattern based on patient specific factors includes determination by a surgeon of maximum allowable incision length. The maximum allowable incision length may be constrained by anatomical location, patient age, or tissue quality. These are particularly relevant in elderly patients or those with compromised healing capacity. The surgeon may also determine acceptable spacing between adjacent incisions, balancing expansion capacity against mechanical stability and graft handling.

The determining of feasible cutting patterns based on the defined cutting pattern limits, evaluated wound site dimensions, and available donor skin includes using the target expansion ratio, a maximum incision length, and spacing constraints as inputs. One or more reference tables, which may be pre-programmed, are consulted to identify all valid combinations of geometric parameters (L, d, g) that satisfy the specified requirements. Each cutting pattern represents a clinically implementable cutting strategy, with associated total incision length and incision count reported to inform trade-off decisions. Cutting patterns with shorter total incision length or fewer incisions may reduce healing burden, while those maximizing expansion efficiency minimize donor tissue requirements.

The selecting of the cutting pattern includes review by a surgeon of the feasible options for the cutting patterns. The surgeon selects the configuration best suited to the clinical scenario. For patients where minimizing healing burden is paramount, cutting patterns with fewer or shorter incisions may be prioritized despite requiring slightly more donor tissue. Conversely, when donor skin is limited, cutting patterns maximizing expansion can be prioritized.

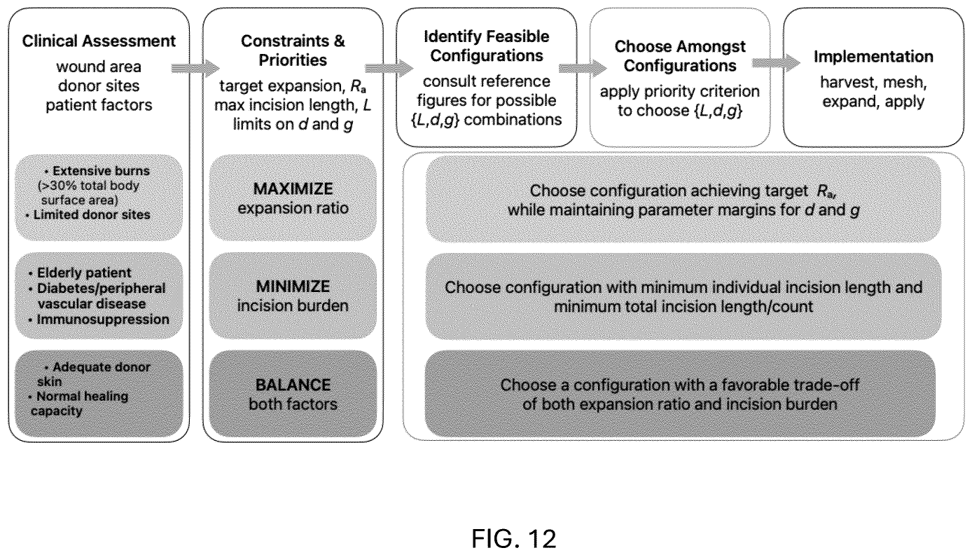

FIG. 12 illustrates a method of meshed skin graft planning. The surgeon inputs wound and donor site assessment, target expansion ratio, and geometric constraints. The model identifies feasible cutting configurations, from which the surgeon selects based on clinical priorities. FIG. 12 illustrates a method including a clinical assessment of wound dimensions, donor site availability, and patient healing factors; specification of target expansion ratio and geometric constraints based on clinical priorities; identification of feasible meshing configurations from reference figures; selection of optimal configuration based on trade-off criteria; and implementation with specified parameters. Priority-based selection guide mapping patient characteristics to optimization approach. Patients with extensive burns and limited donor skin prioritize maximizing expansion ratio; patients with compromised healing capacity (elderly, diabetic, immunosuppressed) prioritize minimizing incision burden; patients with adequate donor skin and normal healing capacity balance both factors. The selection criterion follows directly from the identified priority.

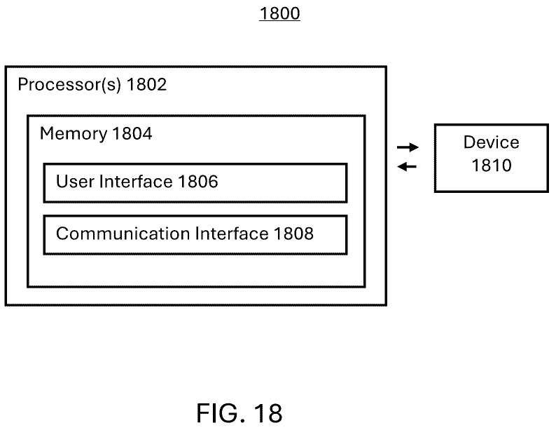

The above method can be implemented by a computer system or integrated system such as that represented in FIG. 18, which illustrates a block diagram of an exemplary computer system 1800. The computer system 1800 may be implemented as a standalone workstation, a tablet computer, or be integrated into a larger surgical system, such as the control system for the smart meshing tool separately disclosed herein. The system 1800 includes at least one processor 1802, a memory 1804, a user interface 1806, and a communication interface 1808. The processor 1802 can be a central processing unit (CPU), a graphics processing unit (GPU), a microcontroller, or any other suitable processing device. The memory 1804, which may include volatile memory (e.g., RAM) and non-volatile memory (e.g., ROM, flash memory, a hard disk drive, a solid-state drive), stores machine-executable instructions and data, such as reference tables. The user interface 1806 may include a display (e.g., LCD screen) for presenting information to a user, such as a surgeon, and an input device (e.g., a touchscreen, keyboard, or mouse) for receiving user inputs. The communication interface 1808 allows the system 1800 to communicate with other devices 1810, such as the smart meshing tool (and associated sensors and/or actuators), and/or with other systems, such as systems that store patient-specific data (e.g., a hospital network). In some examples, system 1800 further includes a server including the processor 1802. The server may communicate patient specific data to the device 1810 through a hospital network.

In a specific embodiment, the memory 1804 stores a predictive modeling module comprising instructions that, when executed by the processor 1802, configure the system 1800 to perform the method of optimizing skin graft expansion. In operation, the processor 1802 receives, via the user interface 1806, inputs from a surgeon including the target expansion ratio, maximum incision length, and spacing constraints between adjacent incisions. The processor 1802 then executes the predictive modeling module to programmatically determine a plurality of feasible cutting patterns by calculating valid combinations of geometric parameters (L, d, g) that satisfy the inputted requirements. In doing so, pre-programmed reference tables stored in memory 1804 may be consulted to identify such valid combinations. For each feasible cutting pattern, the processor 1802 may further calculate associated metrics, such as the total incision length and the total number of incisions. The processor 1802 then causes the user interface 1806 to display the feasible cutting patterns and their associated metrics, enabling the surgeon to review and consider the trade-offs in selecting an optimal cutting pattern. Upon receiving a selection, the processor 1802 may store the selected pattern or transmit its parameters via the communication interface 1808 to another device, such as a connected smart meshing tool for implementation.

### Method and Associated System for Minimizing Secondary Contracture

In one aspect the present disclosure relates to a method for minimizing secondary contracture in split-thickness skin graft surgeries. The method includes selecting, from an integrated system, a desired expansion ratio for a specific skin graft expansion in a patient; receiving, by the integrated system, an input of a set of geometric constraints; determining, by the integrated system, a first set of cutting patterns based on the desired expansion ratio and the input set of geometric constraints, wherein the first set of cutting patterns satisfies both the desired expansion ratio and the set of geometric constraints; and outputting, by the integrated system, the first set of cutting patterns, the first set of cutting patterns including a cutting pattern including one or more of a parallel cutting pattern or a complex geometrical cutting pattern.

The above method can be implemented by a computer system or integrated system such as that represented in FIG. 18, which illustrates a block diagram of an exemplary computer system 1800. The computer system 1800 may be implemented as a standalone workstation, a tablet computer, or be integrated into a larger surgical system, such as the control system for the smart meshing tool separately disclosed herein. The system 1800 includes at least one processor 1802, a memory 1804, a user interface 1806, and a communication interface 1808. The processor 1802 can be a central processing unit (CPU), a graphics processing unit (GPU), a microcontroller, or any other suitable processing device. The memory 1804, which may include volatile memory (e.g., RAM) and non-volatile memory (e.g., ROM, flash memory, a hard disk drive, a solid-state drive), stores machine-executable instructions and data, such as reference tables. The user interface 1806 may include a display (e.g., LCD screen) for presenting information to a user, such as a surgeon, and an input device (e.g., a touchscreen, keyboard, or mouse) for receiving user inputs. The communication interface 1808 allows the system 1800 to communicate with other devices 1810, such as the smart meshing tool (and associated sensors and/or actuators), and/or with other systems, such as systems that store patient-specific data (e.g., a hospital network). In some examples, system 1800 further includes a server including the processor 1802. The server may communicate patient specific data to the device 1810 through a hospital network.

In a specific embodiment, the memory 1804 stores a predictive modeling module comprising instructions that, when executed by the processor 1802, configure the system 1800 to perform the method of minimizing secondary contracture in split-thickness skin graft surgeries. In operation, the processor 1802 receives, via the user interface 1806, inputs from a surgeon including the target expansion ratio, maximum incision length, and spacing constraints between adjacent incisions. The processor 1802 then executes the predictive modeling module to programmatically determine a plurality of feasible cutting patterns by calculating valid combinations of geometric parameters (L, d, g) that satisfy the inputted requirements. In doing so, pre-programmed reference tables stored in memory 1804 may be consulted to identify such valid combinations. For each feasible cutting pattern, the processor 1802 may further calculate associated metrics, such as the total incision length and the total number of incisions. The processor 1802 then causes the user interface 1806 to display the feasible cutting patterns and their associated metrics, enabling the surgeon to review and consider the trade-offs in selecting an optimal cutting pattern. Upon receiving a selection, the processor 1802 may store the selected pattern or transmit its parameters via the communication interface 1808 to another device, such as a connected smart meshing tool for implementation.

### EXAMPLES

### Preliminary Example 1

The parameters L, d, and g were varied to identify all configurations producing target expansion ratios of 1.3, 1.5, 2, and 3. These values span the range commonly employed in clinical practice. Initial graft geometry was assumed to be square, with areas ranging from 4 to 144 cm² (side lengths 2-12 cm) to cover clinically relevant donor sizes. The parameters were varied in discrete increments selected for surgical practicality. Incision length was limited to L<2.5 cm based on instrument constraints and handling considerations. Spacing parameters d and g were bounded within ranges consistent with commercial meshing devices and manual cutting techniques.

Cutting patterns were excluded if they violated any of the following four constraints: (1) geometric overlap between adjacent incisions; (2) spacing values exceeding clinically practical limits; (3) failure to produce a minimum number of incisions (e.g., 3 incisions) in both horizontal and vertical directions; or (4) inability to achieve the target expansion ratio within admissible parameter ranges. The models are not applicable if the incisions are short compared to the thickness of the graft, or are sufficiently sparse that they behave as individual cracks rather than as a networked mesh.

For each valid cutting pattern, the total incision length (sum of all incision lengths) and the total number of incisions were calculated. These metrics enable evaluation of trade-offs relevant to healing outcomes. For example, cutting patterns with shorter total incision length or fewer incisions may reduce healing burden, while those maximizing expansion efficiency minimize donor tissue requirements.

The following clinical case examples illustrate application of the above method (and associated system).

### Example 1: Major Burn Patient with Limited Donor Sites

The method for optimizing skin graft expansion may be applied to patient data regarding a patient who sustained flame burns in an accident as described in Pripotnev S, Papp A. Split thickness skin graft meshing ratio indications and common practices. Burns 2017; 43:1775-81. The initial assessment reveals mixed partial and full-thickness burns covering 52% total body surface area (“TBSA”), with 26% full thickness burn TBSA, concentrated in the trunk and lower extremities, with significant injury in the upper extremity. The posterior trunk and lower legs are unburned and available as donor sites.

Wound requirements are that following staged excision, the surgical team plans definitive autografting of 2,800 cm² of excised wound bed in the current operative session (approximately 16% TBSA in a patient with 1.75 m² body surface area). The patient is young, previously healthy, with no comorbidities affecting wound healing. Healing capacity is normal. Donor site availability includes the unburned posterior trunk and lower legs provide approximately 1,600 cm² of harvestable skin. Additional donor sites will be needed for subsequent stages, so minimizing harvest in this session preserves options for future procedures.

The primary priority is to maximize expansion ratio. The ratio of wound area to available donor area (2,800÷1,600=1.75) establishes the minimum required actual expansion ratio. To provide margin for irregular wound geometry and graft handling losses, the surgeon targets Rₐ=2.0.

Geometric constraints relate to standard dermatome settings, which allow harvest of grafts up to 8 cm wide. The surgeon plans to harvest four grafts of approximately 8×50 cm from the posterior thighs (total initial area ˜1,600 cm²). No specific limitations on incision length or spacing are indicated given the patient's normal healing capacity.

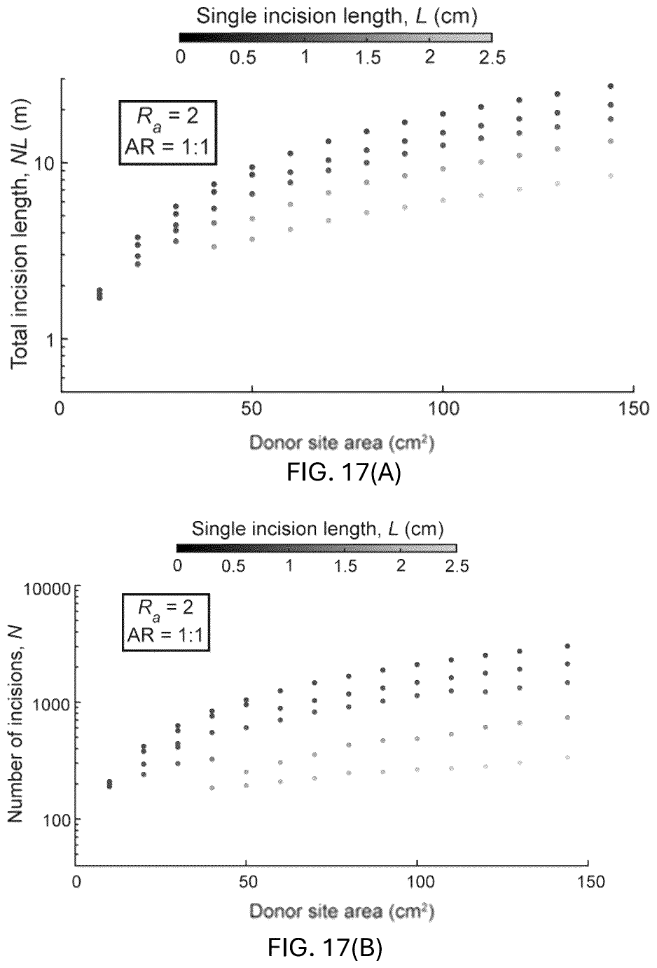

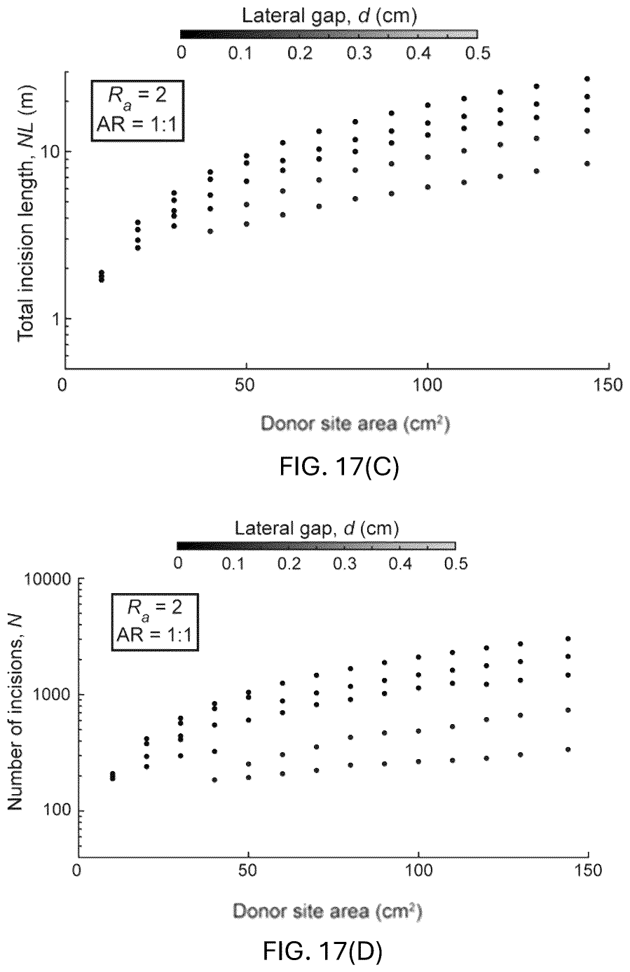

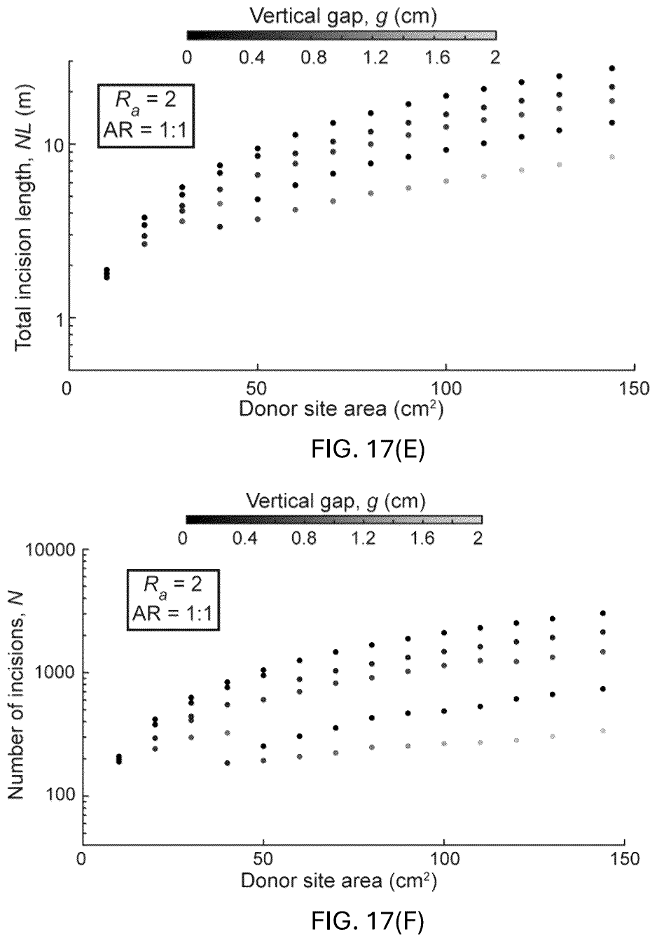

The surgeon consults FIGS. 17(A)-17(F) (expansion ratio 2.0, square geometry approximation by considering 8×8 cm sections). For an initial graft area of 64 cm² per section, three possibilities emerge (Table 1 below).

TABLE 1
Prospective split thickness graft configurations
for the patient of Case 1.

| | | | | Total | Number of |
|---|---|---|---|---|---|
| Configuration | L (cm) | d (cm) | g (cm) | Incision Length | Incisions |
| A | 1.0 | 0.05 | 0.35 | 883 | 883 |
| B | 1.5 | 0.05 | 1.2 | 662 | 441 |
| C | 2.0 | 0.1 | 0.85 | 418 | 209 |

Given the priority of maximizing expansion with no healing-related constraints, the surgeon selects Configuration C (L=2.0 cm, d=0.1 cm, g=0.85 cm). Configuration may refer to a cutting pattern. This achieves the target Rₐ=2.0 with the fewest incisions, although this is a secondary consideration in this case. The longer incision length provides greater opening capacity and remains well within practical limits.

For implementation, the surgeon harvests 1,600 cm² from posterior thighs using an electric dermatome at 0.015 inch thickness. Grafts meshed with the selected parameters and expanded to Rₐ=2.0 are expected to yield 3,200 cm² of coverage, sufficient for the wound bed with appropriate margin for securing and contouring around irregular surfaces.

Using a commercial 3:1 mesher, the surgeon expects to cover 4,800 cm² based on the nominal ratio. However, the actual expansion achieved would be only ˜53% of nominal, yielding approximately 2,550 cm², insufficient for the 2,800 cm² wound bed. This would necessitate either harvesting additional donor skin (depleting reserves for future stages) or leaving wound areas temporarily covered with allograft. By using a better estimate of the actual expansion ratio rather than the nominal ratio, the surgical team has greater potential to achieve complete wound coverage in a single session without depleting donor reserves needed for subsequent stages.

### Example 2: Elderly Patient with Compromised Healing

The method for optimizing skin graft expansion may be applied to patient data regarding a 72-year-old female with type 2 diabetes mellitus, peripheral vascular disease, and chronic corticosteroid use for rheumatoid arthritis presents with a chronic venous stasis ulcer of the left medial lower leg that has failed conservative management for 8 months as described in Tzaneva S, Heere-Ress E, Kittler H, et al. Surgical treatment of large vascular leg ulcers: a retrospective review evaluating risk factors for healing and recurrence. Dermatol Surg 2014; 40:1240-8. Ni Annaidh A, et al. Ann Biomed Eng 2012; 40:1666-78. The patient is described to be scheduled for split-thickness skin grafting following wound bed preparation.

The wound requirements include a 6×6 cm ulcer (36 cm² wound area) with a well-prepared granulating bed following negative pressure wound therapy. The patient has multiple healing-impairing comorbidities: diabetes affects inflammatory response and collagen synthesis, peripheral vascular disease limits perfusion to the graft site, and chronic corticosteroid use impairs all phases of wound repair. Healing capacity is therefore significantly compromised. Donor site availability is not limiting; the ipsilateral or contralateral thigh can provide adequate tissue. Given her age and comorbidities, donor site healing is also expected to be prolonged.

The primary priority is to minimize incision burden. Although donor skin is available, the patient's compromised healing suggests that reducing the total length and number of incisions in the graft may decrease the healing challenge at the recipient site and reduce the risk of graft failure. A modest expansion ratio of Rₐ=1.3 is targeted, requiring harvest of approximately 28 cm² (e.g., a 6×5 cm graft).

Geometric constraints are informed by the patient's healing status. Shorter incisions are preferred to reduce the number of wound edges requiring epithelialization. The surgeon wishes to also minimize the total number of incisions while still achieving the target expansion.

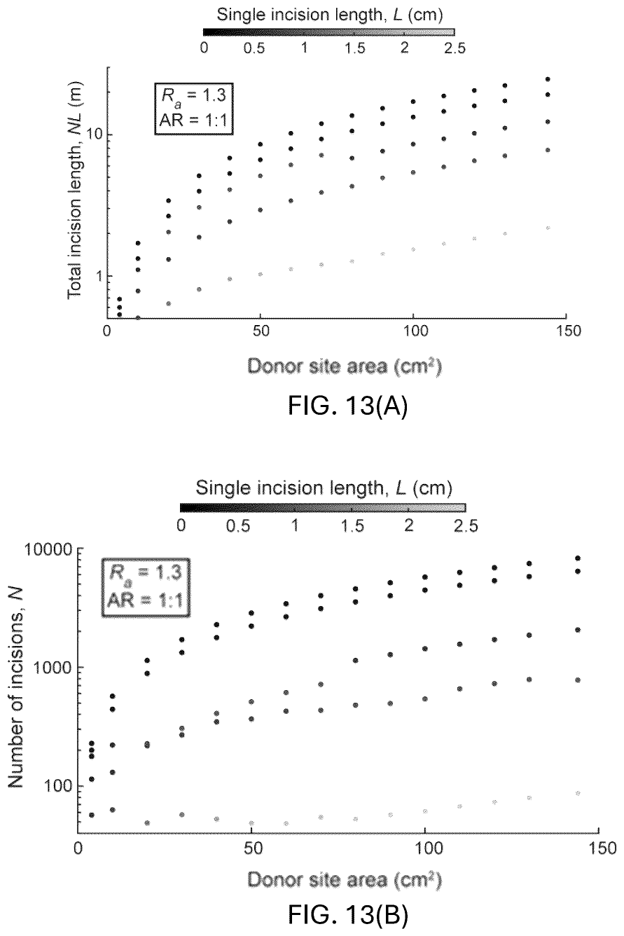

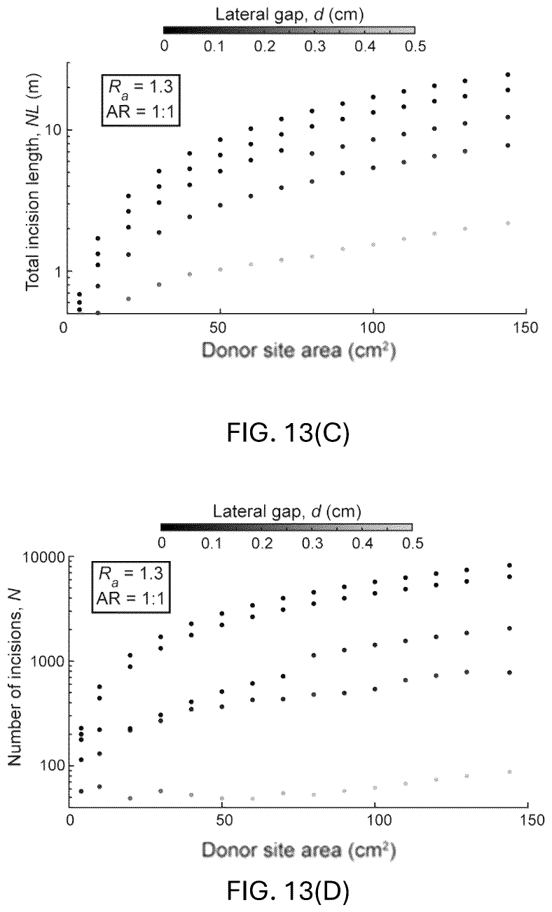

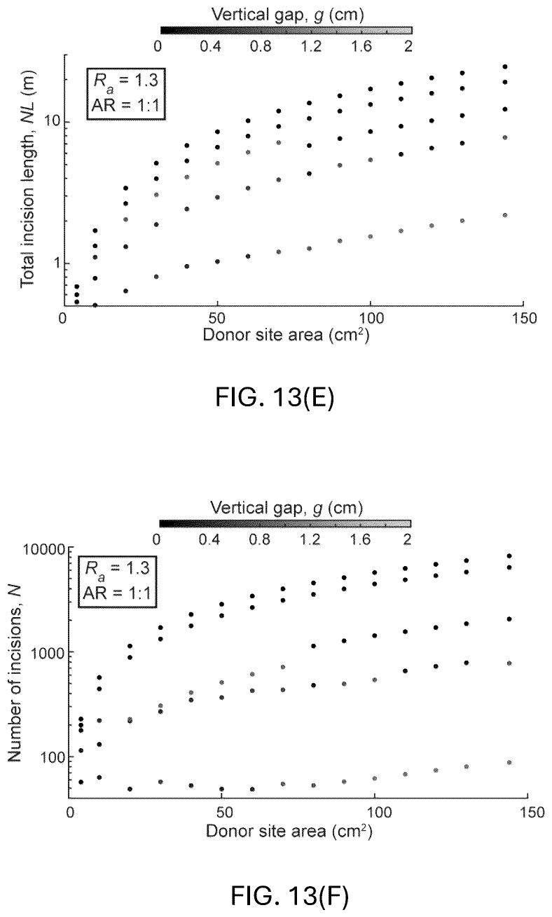

The surgeon consults FIGS. 13(A)-13(F) (expansion ratio 1.3, square geometry approximation). For an initial graft area of 36 cm², three configurations emerge (Table 2 below). Configuration A uses short, densely packed incisions, producing the highest incision count. Configuration C uses longer, more widely spaced incisions, resulting in 33% less total incision length (90 cm vs. 135 cm) and 61% fewer incisions (53 vs. 135) compared to Configuration A. Configuration may refer to a cutting pattern.

TABLE 2
Prospective split thickness graft configurations
for the patient of Case 2.

| | | | | Total | Number of |
|---|---|---|---|---|---|
| Configuration | L (cm) | d (cm) | g (cm) | Incision Length | Incisions |
| A | 1.0 | 0.15 | 0.45 | 135 | 135 |
| B | 1.3 | 0.25 | 0.3 | 93 | 71 |
| C | 1.7 | 0.3 | 0.15 | 90 | 53 |

Given the priority of minimizing incision burden, the surgeon selects Configuration C (L=1.7 cm, d=0.3 cm, g=0.15 cm). This achieves the target Rₐ=1.3 with the fewest incisions and shortest total incision length among available options. The longer incision length remains well within practical limits and is acceptable given adequate tissue handling.

For implementation, the surgeon harvests a 6×5 cm graft (30 cm²) from the anterolateral thigh using an electric dermatome at 0.012 inch thickness (thinner to reduce donor site healing burden). The graft may be meshed with the selected parameters, expanded to Rₐ=1.3, and applied to the prepared wound bed with a non-adherent dressing and negative pressure wound therapy to optimize graft take.

Using a commercial 1.5:1 mesher, the surgeon harvests a smaller graft expecting 1.5× expansion. However, actual expansion would be only approximately 75% of nominal, yielding Rₐ≈1.13, potentially insufficient for the wound. More importantly, the commercial device provides no option to select among configurations with different incision characteristics. By using the reference tables, the surgeon would have explicitly chose the configuration minimizing incision burden (Configuration C over A), reducing total incision length by 33% and incision count by 61%. For a patient with multiple healing-impairing comorbidities, this reduction in the graft's intrinsic healing demand may improve the likelihood of successful take and reduce time to complete epithelialization.

### CONCLUSIONS

A model was developed to predict the expansion ratio Rₐ of meshed split-thickness skin grafts based on the physical mechanisms underlying their expansion. Finite element simulations demonstrate that the conventional model, which assumes each slit transforms into a perfect square without skin stretching, fails to accurately predict the actual expansion ratio. The proposed model captures the two primary mechanisms of skin graft expansion: rotation of the incision arms and stretching of the skin. The proposed model provides upper and lower limits for the expansion ratio as a function of the graft extension ratio, with the upper limit accounting for both rotation and skin stretching, and the lower limit considering only rotation.

The proposed model predicts that surgeons will stop expanding the graft at an extension ratio where the limits diverge, as further expansion beyond this point would require substantial force due to skin stretching. Comparing the proposed model's predictions with experimental data validated its accuracy across different meshing ratios and donor sizes. The proposed model also explains the discrepancy between observed expansion ratios and the nominal expansion ratios associated with the conventional model, showing that grafts with higher nominal ratios achieve a lower percentage of their nominal expansion due to earlier divergence of the limits.

The proposed model's ability to predict the actual expansion ratio based on the meshing parameters offers the potential to guide skin grafting procedures and optimize outcomes. By elucidating the mechanisms of skin graft expansion and providing a validated predictive model, this study addresses a critical gap in understanding and quantifying the behavior of meshed split-thickness skin grafts.

The parameter variations of Preliminary Example 1 identified multiple feasible cutting patterns or configurations for each target expansion ratio across the range of initial graft sizes examined. Across all expansion ratios, several consistent patterns emerged. Total incision length and total number of incisions both scale with initial graft area, as expected from geometric considerations. For any given target expansion ratio and initial area, surgeons can select from configurations spanning a range of incision lengths and incision counts, enabling patient-specific optimization. Configurations with larger L values generally require greater total incision length but fewer individual incisions, while smaller L values produce the inverse pattern. Higher expansion ratios progressively constrain the feasible design space and require parameter combinations closer to practical limits.

FIGS. 13-17(A)-13-17(F) illustrate feasible parameter configurations for various expansion ratios. FIGS. 13-17(A)-13-17(F) illustrate a total length of incisions (FIGS. 13-17(A), (C), (E)) and total number of incisions (FIGS. 13-17(B), (D), (F)) versus initial graft area. Color coding indicates incision length L (FIGS. 13-17(A), (B)), horizontal spacing d (FIGS. 13-17(C), (D)), and vertical gap g (FIGS. 13-17(E), (F)).

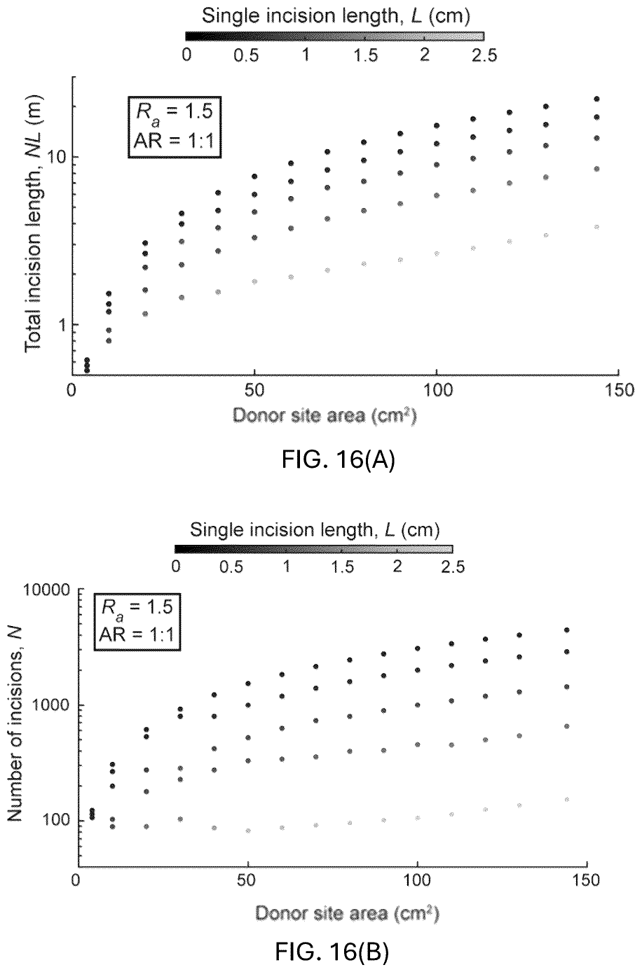

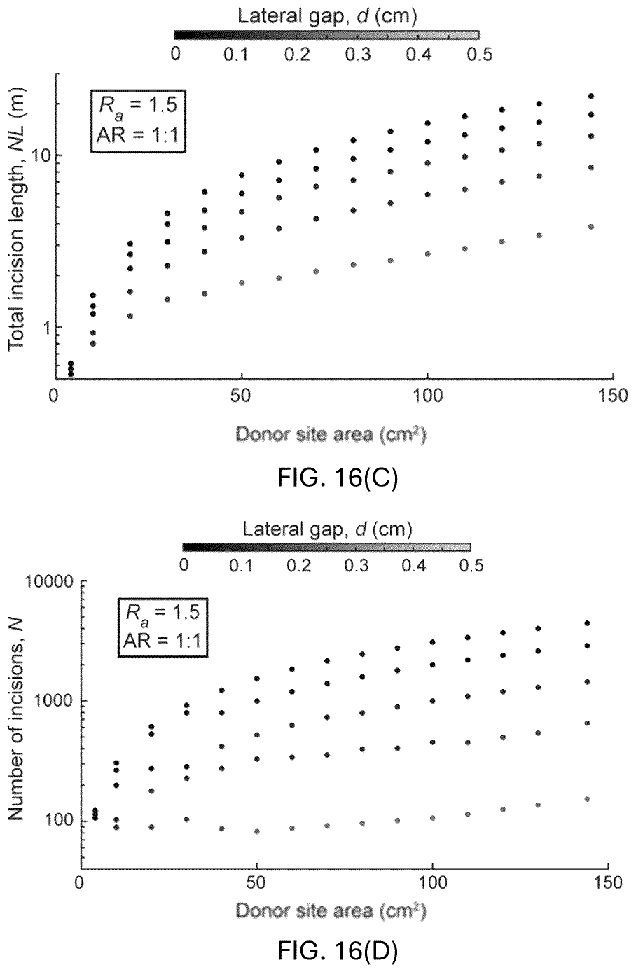

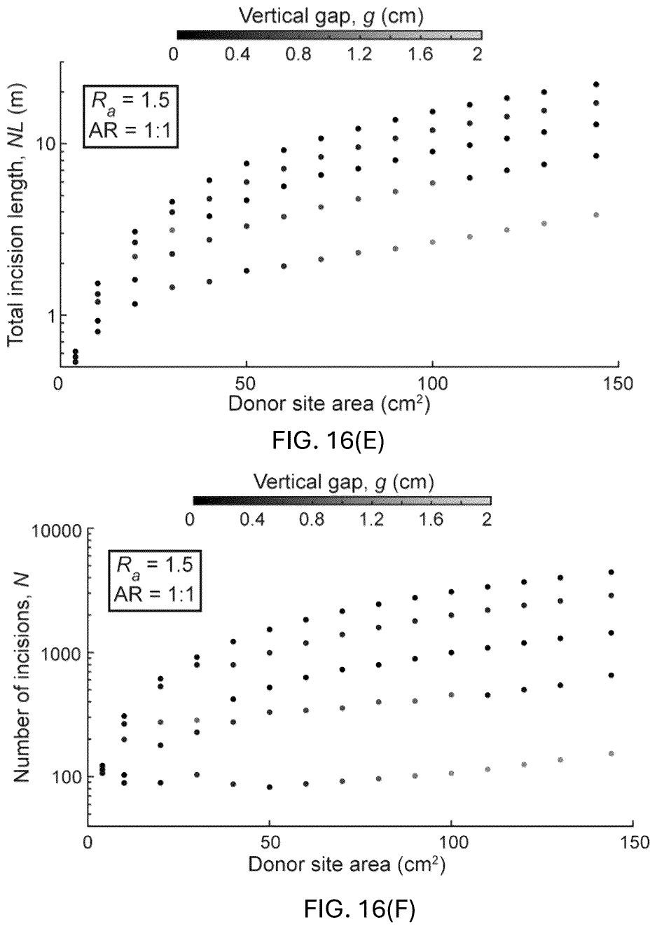

For expansion ratio 1.3, total incision length increased approximately linearly with initial graft area across all valid configurations (FIGS. 13(A), (C), (E)). Multiple valid configurations exist for each initial area, offering trade-offs between fewer long incisions and numerous short incisions (FIGS. 13(B), (D), (F)). The feasible design space expands at expansion ratio 1.5, with a wider range of valid (L, d, g) combinations available for each initial graft size (FIGS. 16(A)-16(F)). The relationship between total incision length and initial area remains approximately linear, with slope dependent on the specific parameter combination selected. Similar patterns hold for expansion ratio 2 (FIGS. 17(A)-17(F)).

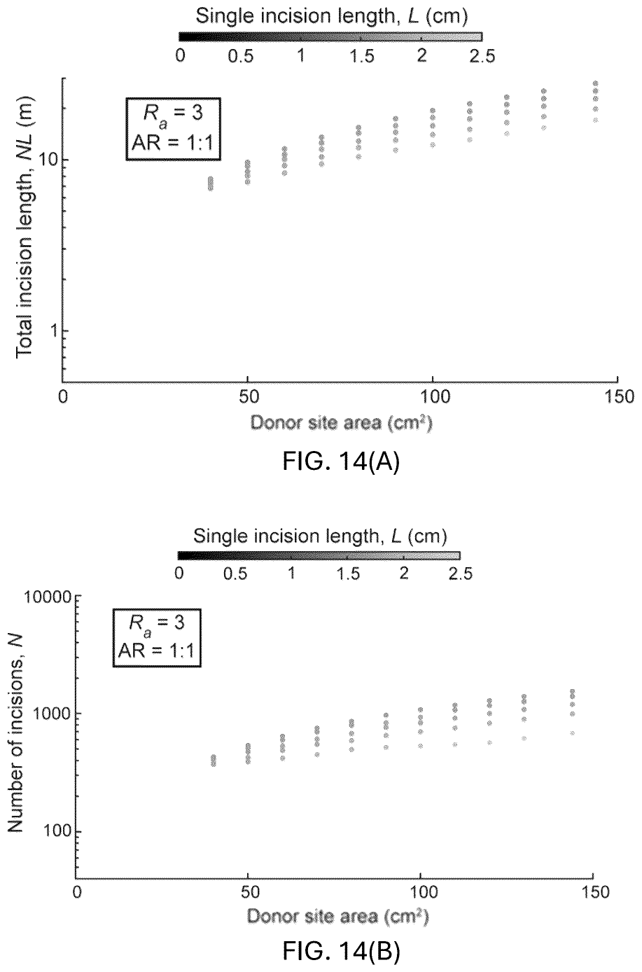

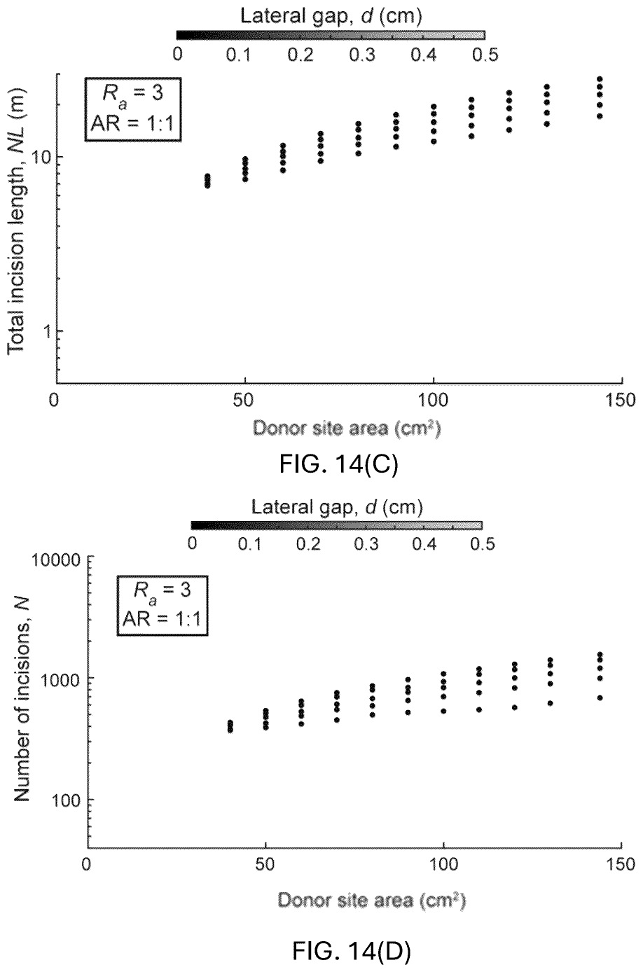

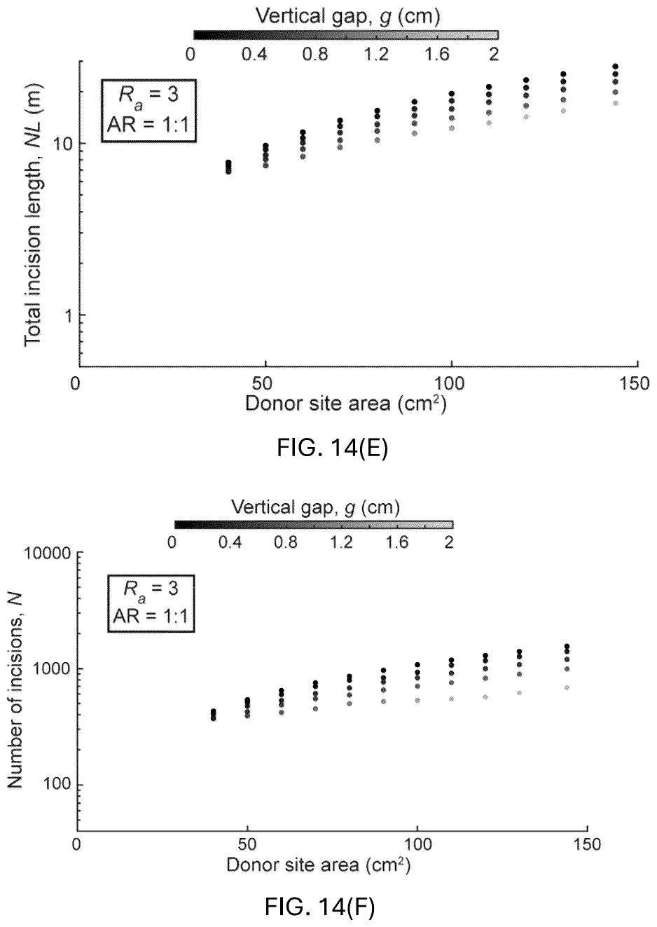

Achieving expansion ratio 3 requires configurations with relatively longer incisions or tighter spacing compared to lower ratios (FIG. 14(A)-14(F)). The number of feasible configurations for smaller graft sizes becomes substantially more constrained at this ratio, reflecting the geometric requirements for achieving greater expansion. Fewer valid configurations exist, particularly for smaller initial graft areas, and the required parameter combinations approach the limits of surgical practicality. The trade-off between incision length and incision number becomes more pronounced at this high ratio, with configuration choice having greater impact on total incision burden.

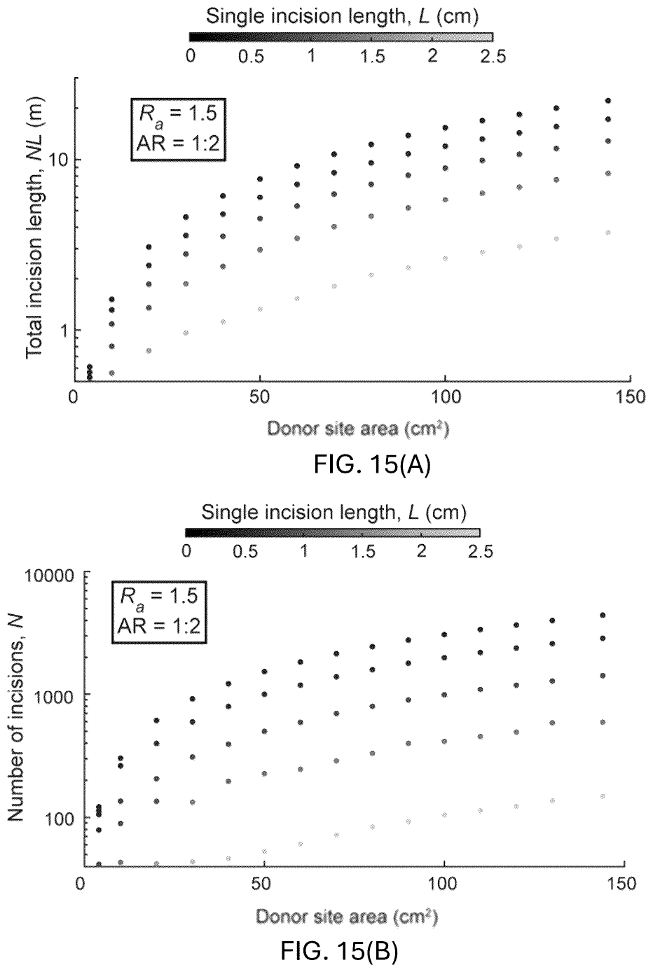

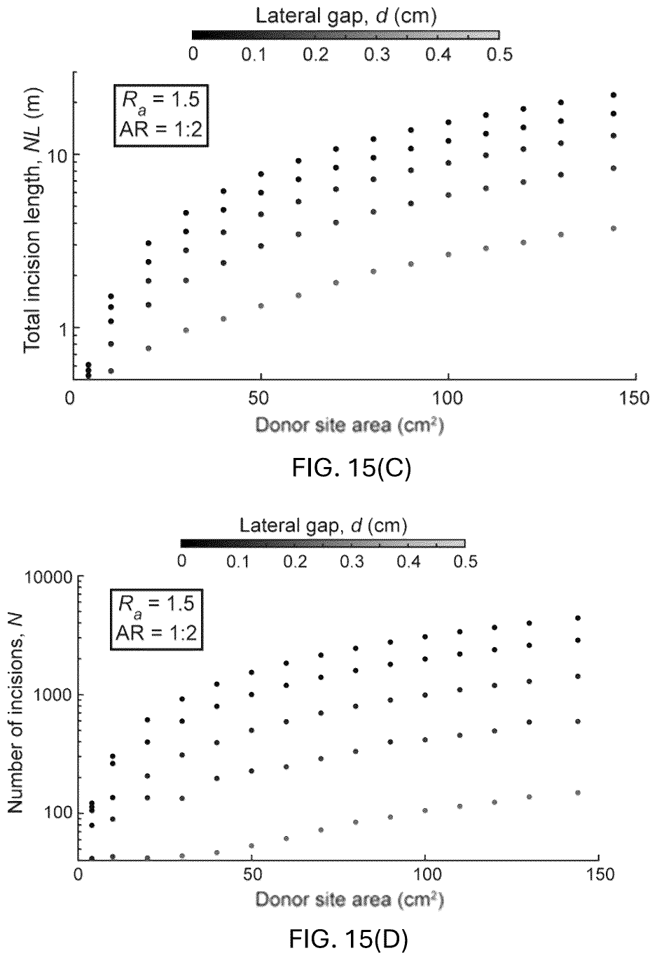

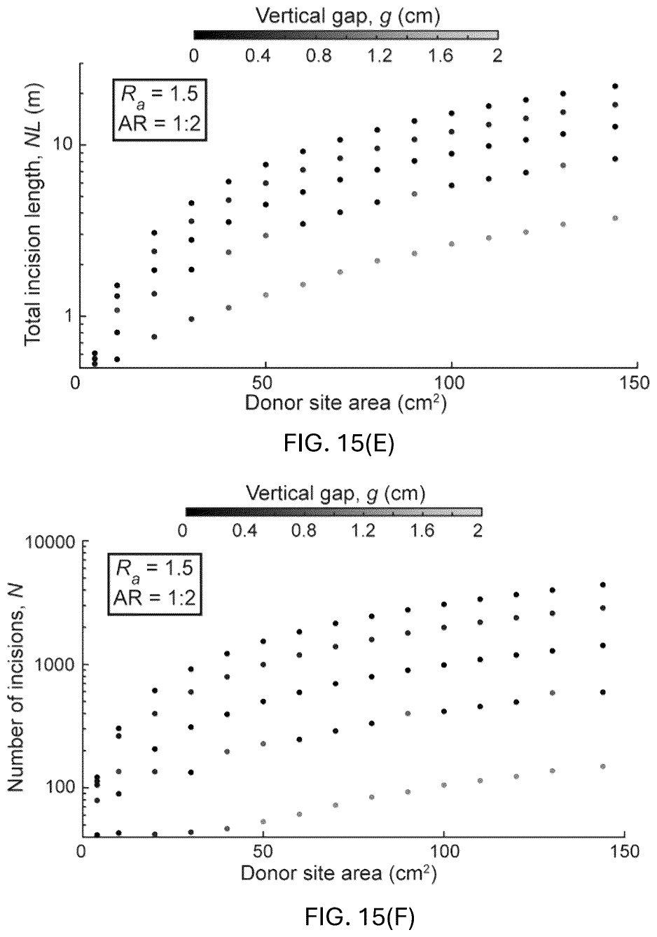

The results presented above assume square initial graft geometry. To examine the effect of graft shape, the parameters were varied for expansion ratio 1.5 using rectangular grafts with a 2:1 aspect ratio. The feasible design space shifts compared to square grafts of equivalent area, with different combinations of L, d, and g becoming accessible (FIGS. 15(A)-15(F)). This demonstrates that initial graft shape influences the available parameter configurations, and surgeons harvesting non-square donor tissue should account for aspect ratio when selecting meshing parameters.

The different combinations of L, d, and g relate to reference figures for different cutting patterns or configurations. The reference figures enable rapid identification of suitable configurations: surgeons can locate their initial graft size on the horizontal axis, identify the target expansion ratio, and read off the available combinations of L, d, and g along with the associated total incision length and incision count for each option.

Variation of the parameters identified that multiple valid configurations exist for any target expansion ratio and graft size, enabling optimization based on patient-specific factors. The parameter variation also identified that higher expansion ratios progressively constrain the feasible design space. At expansion ratio 3, fewer valid configurations exist and required parameters approach practical limits. When reference figures indicate limited options, surgeons need to consider staged grafting or acceptance of lower expansion with larger donor harvest. The parameter variation further identified that graft geometry influences available configurations. The shift in feasible design space between square and rectangular grafts (FIGS. 15(A)-15(F)) indicates that surgeons harvesting non-square tissue should account for aspect ratio when selecting parameters.

Examples 1 and 2 illustrate how accurate prediction impacts the two patient populations for whom this tool is most consequential. For extensive burns, achieving the predicted expansion determines whether coverage is achieved in one session or requires additional procedures, with direct implications for donor site preservation and survival. For elderly patients, selecting gentler configurations reduced total incision length and incision count, potentially meaningful given healing impairments in this population. The explicit incorporation of patient-specific priorities departs from current practice, which relies primarily on TBSA-based rules applied uniformly. By providing trade-off data for each configuration, the reference figures enable quantitative consideration of healing capacity and comorbidities.

The present disclosure provides a computation-free method for translating a validated biomechanical model into clinical practice. The reference figures and structured workflow enable surgeons to determine minimum donor skin requirements before harvesting, select patient-specific meshing parameters based on explicit trade-offs, and tailor incision patterns to clinical priorities, including maximizing expansion for donor tissue-limited burn patients or minimizing incision burden for healing-compromised elderly patients. By bridging the gap between mechanistic understanding and surgical application, this framework addresses a long-standing clinical need for accurate, practical prediction of skin graft expansion.

### Stretch-Induced Cellular Memory and Fibroblast Activation

Mechanical stretching of living tissues can activate long-lived changes in tissue cells such as fibroblasts, increasing their contractility and initiating phenotypic transformations. Increased mechanical stimulus typically leads to monotonically increasing activation of fibroblasts cultured in 2D, but activation levels are difficult to predict for cells in 3D fibrous tissues, leading to variable outcomes in procedures such as skin grafting. The source of this variation is cell-extracellular matrix (ECM) interactions and their variation with the duration and magnitude of applied stretch. A model can predict the degree to which stretch will either increase or decrease long-term activation levels of fibroblasts cultured within a stretched, three-dimensional collagen matrix. Combining experimental and mathematical approaches across multiple scales, the visco-plasticity of the extracellular matrix (ECM) regulates this nonmonotonic, long-term cell activation. Results demonstrate that feedback between cell and ECM determines how cells retain memory of mechanical stretch.

Soft tissues generally exist in a state of homeostatic tension. During development, wound healing, and surgical procedures such as skin grafting, wound closure, and flap reconstruction, the homeostatic set-point can shift, potentially leading to complications such as fibrosis, hypertrophic scars, keloid scars, and failed skin grafting. These outcomes are difficult to predict, leading to significant patient burden. For example, failed skin grafting is a devastating and unpredictable potential outcome for those of the 8.2 million chronic wound patients in the US each year whose chronic wounds are treated with split-thickness skin grafts. For these patients, chronic wounds arising from burns, diabetic ulcers, skin cancer surgery, or infection require surgical grafting to protect the wound from the environment and pathogens. The skin graft consists of epidermis and a thin variable layer of dermis from a donor site that is harvested meshed with an array of slits, stretched, and then transplanted over a larger wound site. Skin grafts placed on wound sites contract due to forces generated by dermal fibroblast cells from the donor site that are transferred within the graft. The graft can fail if this long-term contraction is either too great or too small, leading to complications ranging from poor aesthetic outcomes to major functional limitations such as scar contracture that impairs the function of joints such as the ankle, axilla, elbow, and wrist. However, the level of this critical long-term contractility currently cannot be predicted. Levels of long-term graft contractility were hypothesized to arise from activation or deactivation of graft fibroblasts that can be predicted from the magnitude and duration of the tensile strains that are transmitted to them through the extracellular matrix (ECM).

The rationale for this hypothesis is the apparent propensity of many cells to seemingly remember their previous mechanical conditions through changes to gene or protein expression that persist over physiologically relevant timescales. An example is mesenchymal stem cells cultured on two-dimensional (2D) substrata, wherein memory of substrate stiffness affects subsequent cell differentiation, and can persist even after cells are moved to a new substrate of different stiffness. Cyclic dynamic stretching, as occurs in development, also induces mechanical memory in stem cells, but static stretches do not. Fibroblasts are known to remember the stiffness of 2D substrates they encounter, with memory of relatively stiff substrates persisting in lung fibroblasts for weeks, even after cells are returned to softer substrates. In fibroblasts, this memory of a stiff environment manifests as elevated contractility and as activation, wherein fibroblasts express myofibroblast gene and protein expression signatures that are hallmarks of wound healing and fibrotic disease.

This motivation assisted in predicting that such mechano-signaling may have sustained effects in the form of long-term activation or deactivation of fibroblasts. Forces on fibroblasts are often in the form of long-term static stretching or compressing of the tissues in which they reside. In the case of skin grafting, these forces are tensile and are known to vary with the direction of skin stretching relative to the “Langer's lines” that define cell polarity and are associated with surgical incision directions that reduce scarring. However, it remains unknown whether fibroblasts can develop and maintain mechanical memory under static loading. Furthermore, these external and long-term static forces are transmitted to fibroblasts through their three-dimensional (3D) extracellular environment whose physical properties are known to change with the magnitude and duration of the external loading.

### Results

Sustained stretch of tissues over a clinically relevant range of strains leads to dramatically different homeostatic states depending upon strain magnitude and duration.

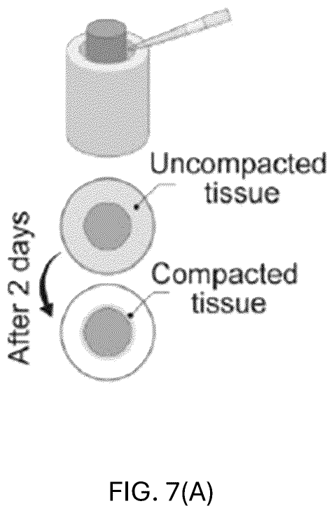

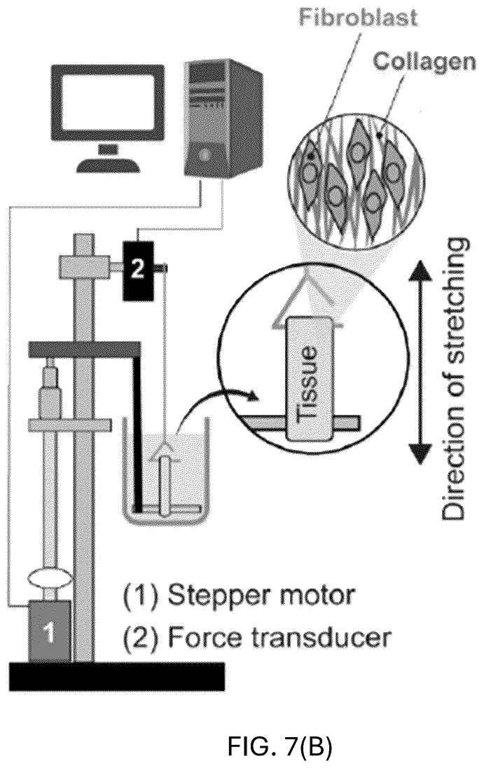

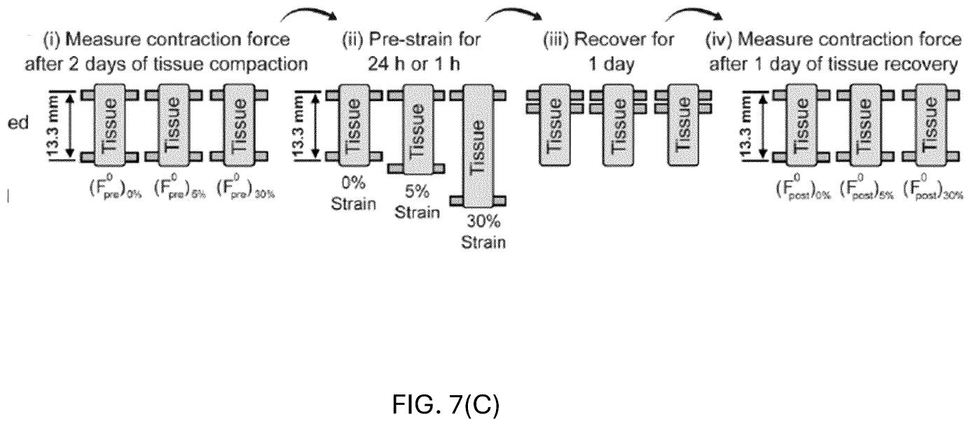

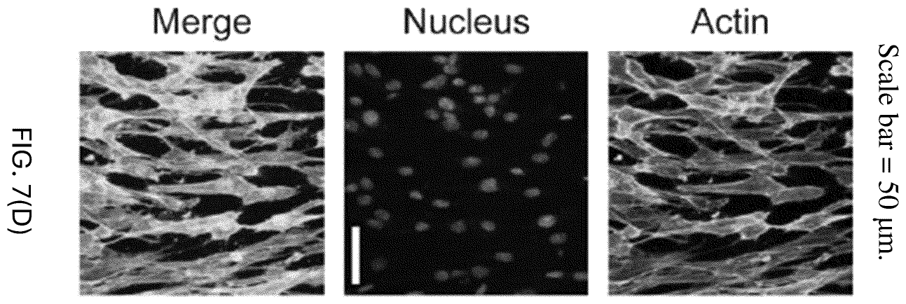

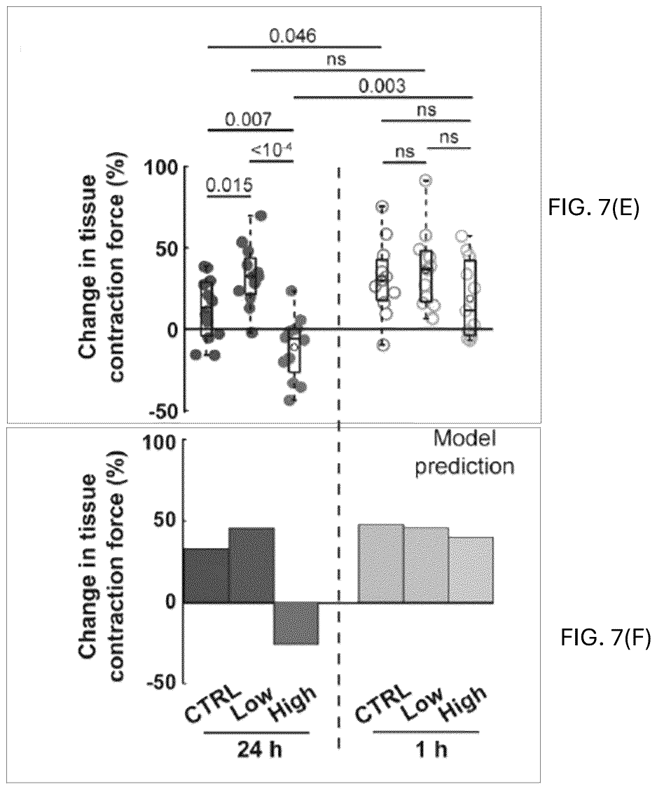

To quantify the effect of sustained stretch on fibroblast activation, the contraction of synthetic tissues containing human dermal fibroblasts and type I collagen were studied. FIG. 7(A) illustrates ring-shaped tissue constructs, consisting of human dermal fibroblasts and type I rat tail collagen, that were removed from molds after two days. Thin (˜0.1 mm thickness), ring-shaped specimens formed as the fibroblasts contracted the collagen onto the central cylinder of an annular Teflon mold over two days (FIG. 7(A)). Thin specimens were chosen to represent certain conditions of split-thickness skin grafting, with reduced graft thickness often correlating with excessive contractility.

The resulting ring-shaped tissues were placed on a uniaxial stretching apparatus and subjected to a prescribed stretching regimen. FIG. 7(B) illustrates tissue placed on a force measurement device. Part (i) of FIG. 7(C) illustrates isometric contractile force (F⁰)ₚᵣₑ was measured in the reference configuration (inner perimeter of tissue equaling the inner circumference of the mold's central cylinder). In part (ii) of FIG. 7(C) tissues were strained 0% (CTRL), 5% (low), or 30% (high) and held for 24 h or 1 h, then in part (iii) of FIG. 7(C) allowed to relax for 1 day. Part (iv) of FIG. 7(C) illustrates post-stretch isometric force (F⁰)ₚₒₛₜ measured in the unstretched reference configuration.

Tissues were stretched with directions, durations, and magnitudes of stretch chosen to represent split-thickness skin grafts. The circumferential direction of loading coincided with the direction of cell alignment (FIGS. 7(B), (D), representative of the stretching of skin grafts along Langer's lines. Tissues that were not in the unstretched control group were stretched with strains of 5% (“low”) or 30% (“high”), representative of average strains associated with the range of typical skin grafts. All tissues were stretched for either one hour or 24 hours, representative of different environments where a skin graft can reside on the human body in a static or dynamic wound environment such as over an immobile (e.g., trunk) or mobile (e.g., joint) recipient bed. FIG. 7(D) illustrates cellular alignment within the tissues mirrored patterns observed along Langer's lines in vivo. Tissues were stretched along the direction of cell alignment.

For each tissue, the isometric tissue contraction force (F⁰)ₚᵣₑ was measured immediately after placing the tissue on the stretching apparatus on day 2 (part (i) of FIG. 7(C)), the tissue was then “pre-strained” by 0% (control), 5% (low), or 30% (high) for either 24 hours (“long duration”) or 1 hour (“short duration”) (part (ii) of FIG. 7(C)). The tissues were then allowed to relax and return to the unstretched baseline length for one day (part (iii) of FIG. 7(C)), after which the isometric tissue contraction force (F⁰)ₚₒₛₜ (part (iv) of FIG. 7(C)) was measured in the unstretched reference configuration. The change in contraction force associated with the strain treatment, ΔF⁰=(F⁰)ₚₒₛₜ−(F⁰)ₚᵣₑ, was recorded.

FIG. 7(E) illustrates change in the tissue contractile force was determined as Φ=((F⁰)ₚₒₛₜ−(F⁰)ₚᵣₑ)/(F⁰)ₚᵣₑ. Short-duration pre-straining had no effect on D, but a biphasic response was observed for long-duration pre-straining, with tissue contractility dropping following high strain (n=12 and 11 for the long-duration and short-duration pre-straining, respectively). FIG. 7(F) illustrate computational simulations using the present disclosure for predicting these results.

For all three specimens stretched for 1 hour, ΔF⁰/(F⁰)ₚᵣₑ was positive, with the increase in contractility independent of stretch (FIG. 7(E)). However, tissues stretched for 24 hours showed a biphasic trend with respect to strain treatment: for tissues subjected to low stretch, ΔF⁰/(F⁰)ₚᵣₑ was positive and maximum, while for tissues subjected to high stretch, the mean ΔF⁰/(F⁰)ₚᵣₑ was negative and significantly lower (p<0.0001, FIG. 7(E)).

The time and strain dependent properties of the ECM regulate changes to cell homeostasis and tissue contractility.

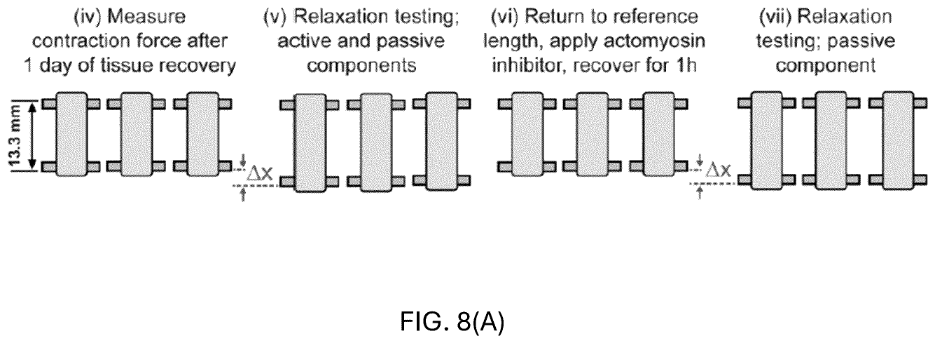

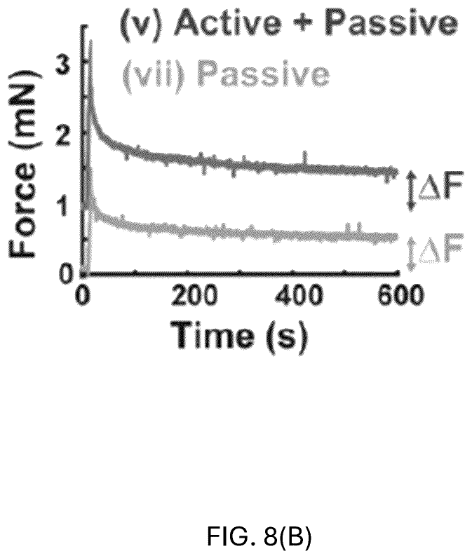

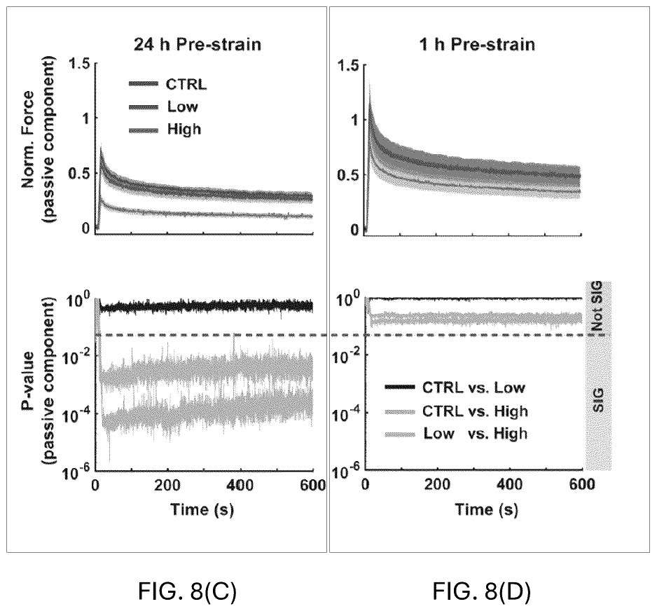

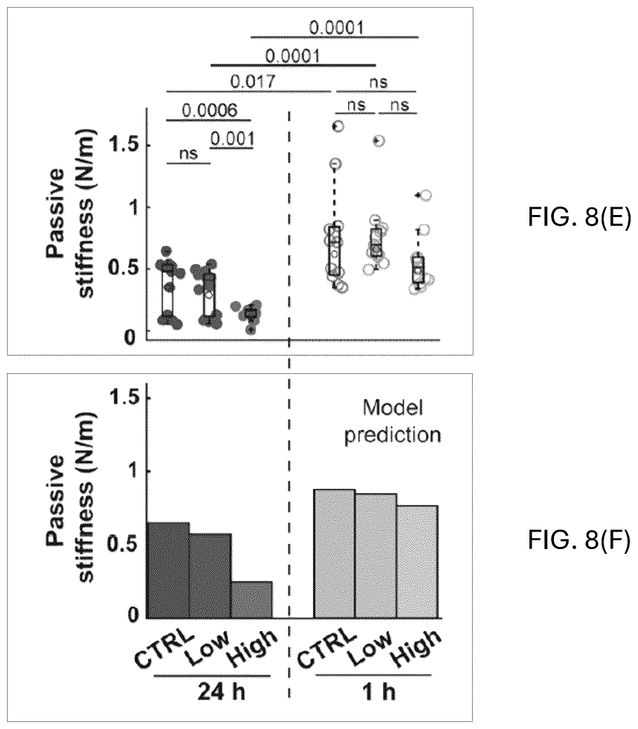

To identify sources of this unexpected bimodal behavior, the active and passive mechanical responses of the tissue specimens were separated. After completing the 24 h of tissue recovery (part (iv) of FIG. 7(C)), viscoelastic stress relaxation testing was performed on each tissue, in which tissues were stretched by an amount ΔX=0.66 mm and held at that length while the isometric force was recorded (part (v) of FIG. 8(A)). The force rose to a peak during the stretching, and the subsequent isometric force then relaxed to an asymptotic value over time (FIG. 8(B)). The difference ΔF between the pre-test isometric force (force measured at 0 s) and the force measured after 600 s of relaxation was recorded, and the long-term stiffness ΔF/ΔX was calculated.

FIG. 8(A) shows effects of pre-strain magnitude and duration on the long-term passive responses of tissues were quantified via stress relaxation tests after the completion of tests described in FIGS. 7(A)-7(F). Isometric stress relaxation tests were conducted on the tissues (part (v) of FIG. 8(A)) and then repeated (part (vii) of FIG. 8(A)) with actomyosin contractility eliminated using cytochalasin D. FIG. 8(B) is a representative stress relaxation response of tissues before (purple line-light shaded line) and after (brown line-darker shaded line) disruption of actomyosin contractility. Long-term stiffness was determined as ΔF/AX. FIGS. 8(C)-(E) illustrate disruption of actomyosin contractility revealed significant softening in tissues pre-strained for 24 h, with softening further increasing with tissue pre-strain magnitude. On the contrary, pre-straining of tissues for 1 h did not cause permanent changes to long-term ECM stiffness (n=13 and 11 for the 24 h and 1 h groups, respectively). In FIG. 8(C) and FIG. 8(D), the solid lines and the shaded areas represent the mean and standard error, respectively. FIG. 8(F) illustrates passive stiffnesses predicted by the active chemo-mechanical tissue model agreed with experimental observations

Tissues were subsequently returned to their reference lengths, treated with the actomyosin contractility inhibitor cytochalasin-D, then allowed to relax for one hour prior to repetition of the viscoelastic relaxation test to obtain the passive response of the tissues (parts vi-vii of FIG. 8(A)). Cytochalasin-D treatment eliminated active contraction at the start of the relaxation test and reduced the peak force, but had little effect on the rate of long-term stress relaxation (FIG. 8(B)).

The present results showed that tissues pre-strained for 24 h (long duration) had significantly softer ECMs compared to tissues pre-strained for 1 h. Furthermore, while the long-term ECM stiffness of tissues pre-strained for 1 h remained independent of pre-strain level, the long-term ECM stiffness of tissues pre-strained for 24 h varied with pre-strain level, with the ECM of tissues pre-strained at 30% for 24 h significantly softer than the ECM of tissues pre-strained at 0% or 5% (FIGS. 8(C)-(E)).

Thus, pre-straining of tissues for 1 h did not cause permanent changes to long-term ECM stiffness, with the ECM showing full elastic recovery, but pre-straining of tissues for 24 h led to ECM softening, with the degree of softening affected by the pre-straining magnitude.

Time and strain dependent ECM behavior regulates the stress levels that cells experience.

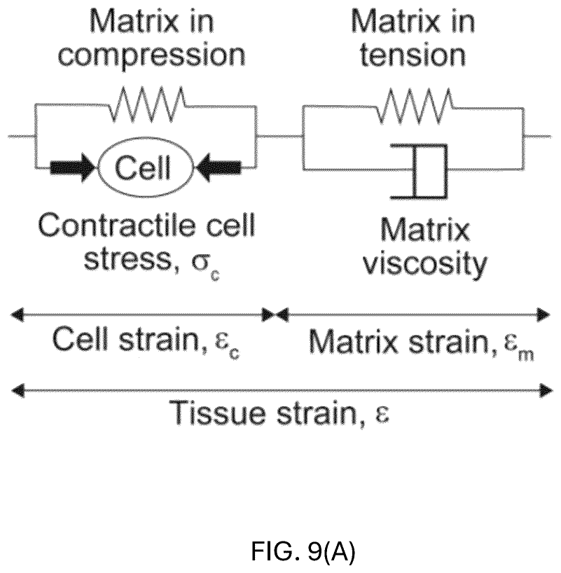

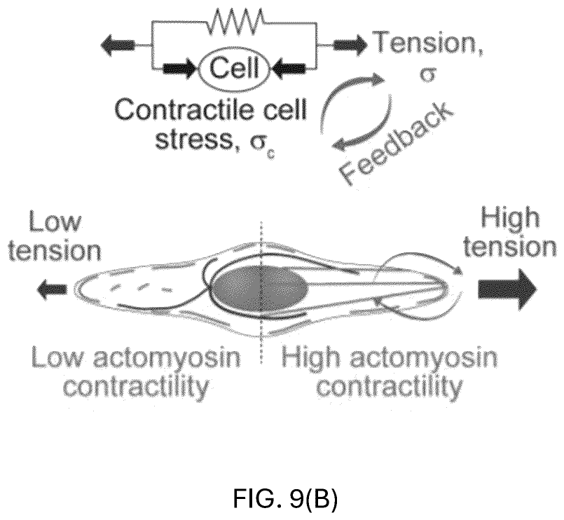

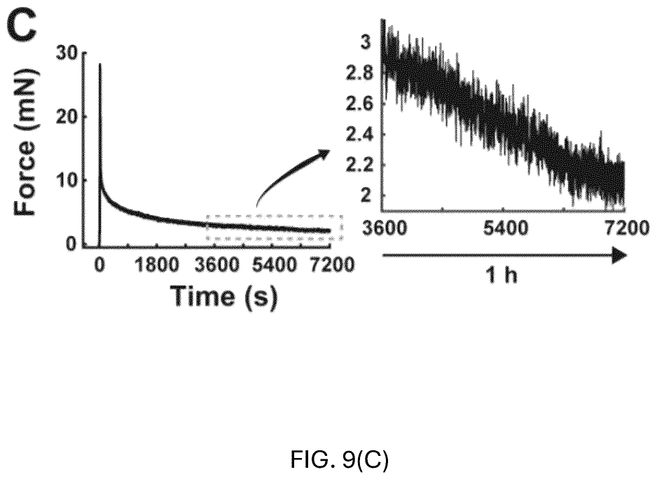

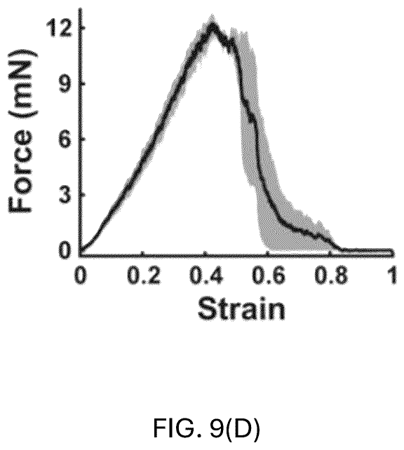

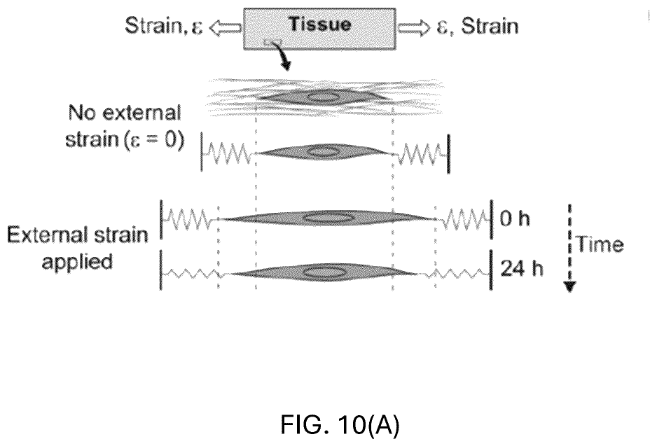

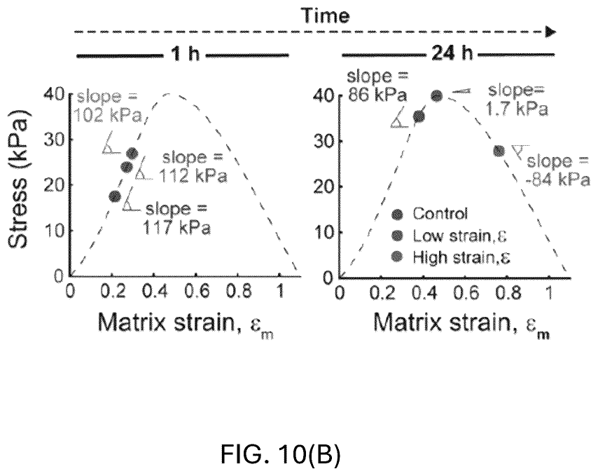

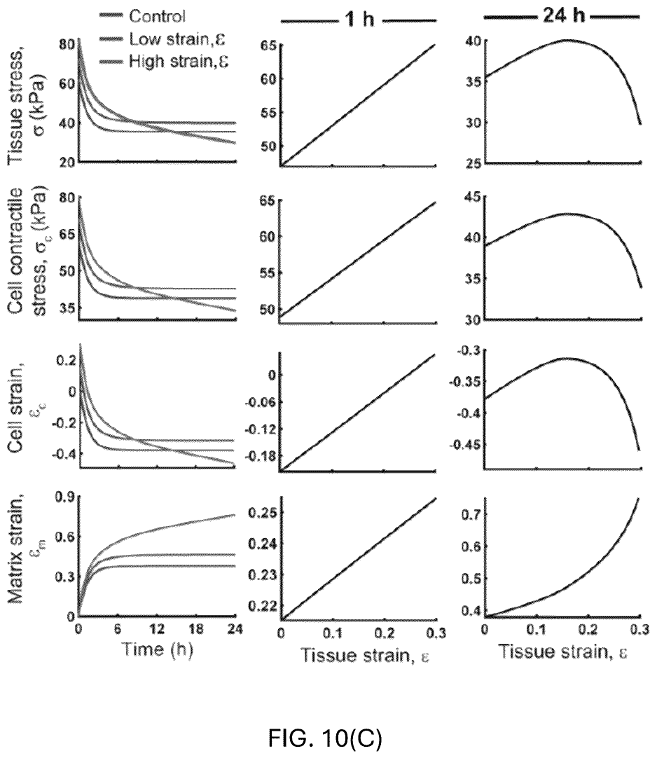

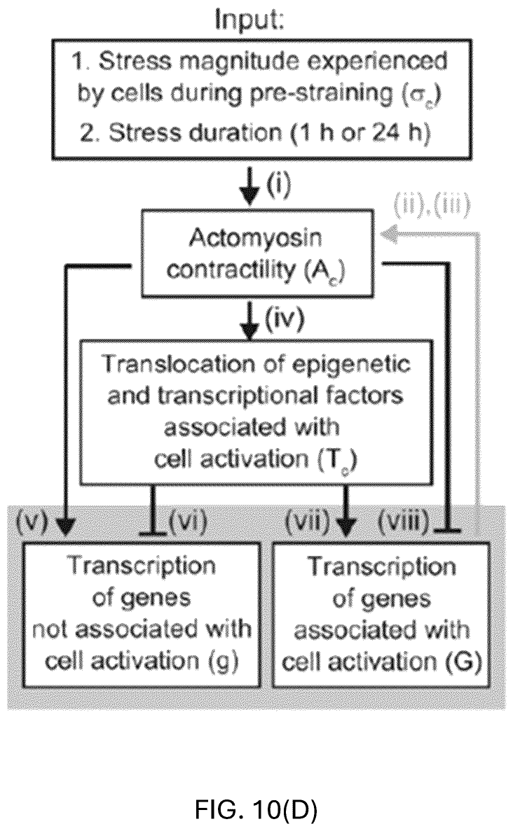

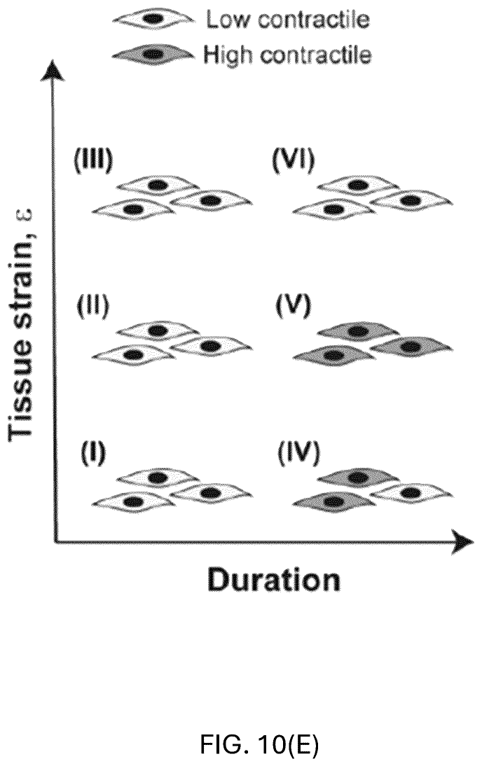

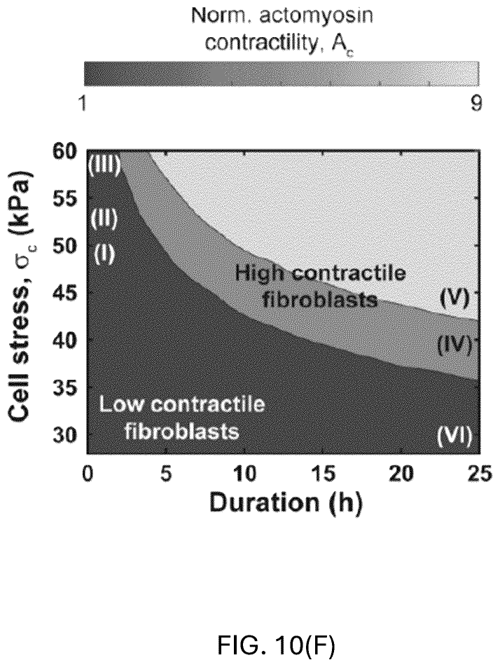

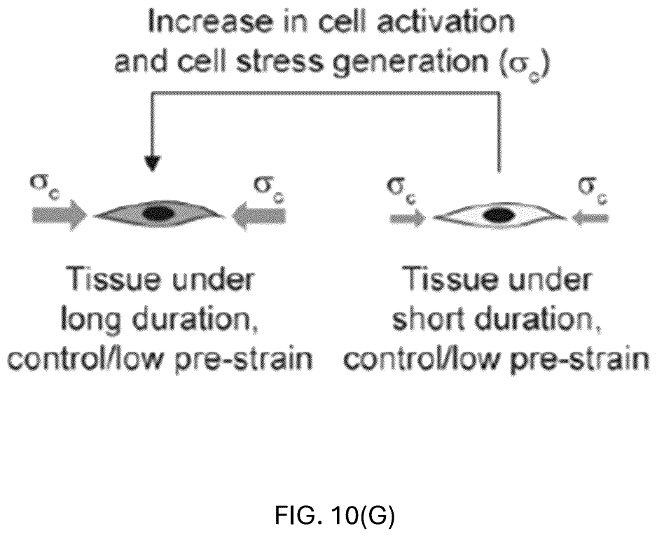

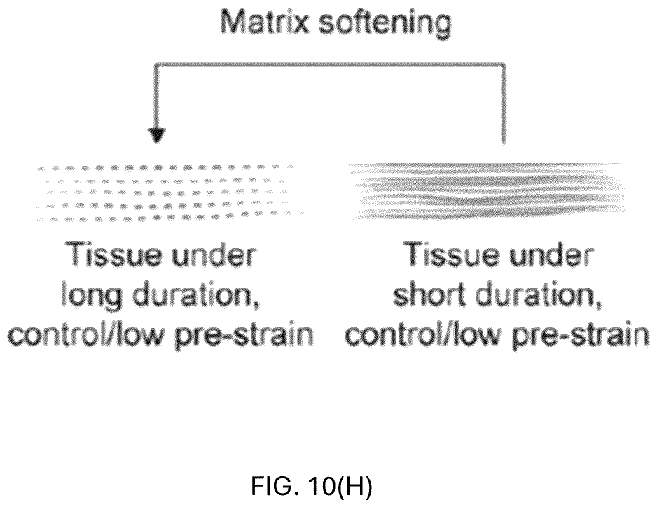

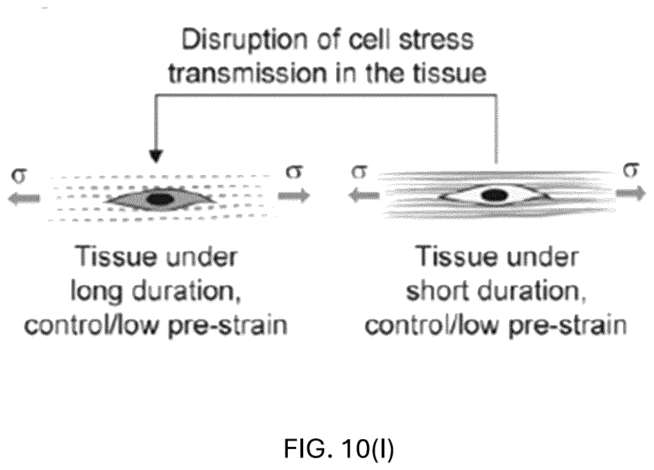

To understand mechanisms underlying the effects of pre-strain duration and magnitude on tissue contractility (FIG. 7(E)-(F)), the passive and active responses of the tissues were linked through a computational model. This included an active chemo-mechanical tissue model (FIG. 9(A)) coupled with a cell signaling network model (FIG. 10(D)). The tissue-level model predicted how forces reach cells through the ECM, and accounted for how ECM properties change with magnitude and duration of stretching. The cell-level signaling model predicted how these forces impact long-term, time-dependent behavior of cells.

FIG. 9(A) illustrates a one-dimensional representation of the active chemo-mechanical tissue model composed of an active force-generating element (cell) connected to two passive elements. In the absence of external strain (ε=0), one element represents regions of the ECM under compression due to cell contraction, and the other represents regions of the ECM in tension. The model represented the following three experimental observations. FIG. 9(B) shows actomyosin contractility and thus cell contractile force increase with tension. FIG. 9(C) shows ECM exhibits long-term relaxation under tension. FIG. 9(D) shows long-term ECM stiffness varies with ECM strain magnitude. The initial increase in the slope of a force-strain curve is followed by a softening region at larger strains (n=3, the solid line and the shaded area represent the mean and standard error, respectively).

The chemo-mechanical tissue model consisted of an ECM model and a cell model endowed with the ability to generate active forces (FIG. 9(A)). The ECM model was based upon two experimental observations. The first was that the ECM's mechanical properties exhibit long-term, time-dependent changes under sustained tension. This hypothesis was supported by passive stress relaxation tests like those of FIGS. 8(C)-(D), but performed at a higher stretch (ΔX=4 mm) and over a much longer timescale (FIG. 9(C)). Results showed that the passive component of the tissue response continued to decrease with time, resulting in more than a 30% drop in matrix stress over 1 h (FIG. 9(C)). This ECM behavior was modeled by adding a viscous element to the ECM response in tension (FIG. 9(A)).

The second was that ECM stiffness changes with magnitude of tensile ECM strain. To evaluate this hypothesis, tissues treated with cytochalasin-D were stretched at a slow strain rate of 3.11 μm/s to minimize the ECM viscous effect. Experiments showed strain-stiffening up to a peak value, followed by a softening region (FIG. 9(D)). This was modeled by incorporating this experimentally measured stress-strain relationship into the constitutive model of the ECM element in tension (FIG. 9(A)).

The cell model was based on the observations that actomyosin contractility and thus cell-generated contractile forces increase with cell tension, including tension arising from external tensile forces and from ECM resistance to cell contraction (FIG. 9(B)). Increased actin stress fiber polymerization occurs in the direction of an applied stretch for cells in a 3D ECM. Increased actomyosin contractility and cell force generation occurs in response to higher matrix stiffness. This was modeled by incorporating cell-generated contractile stress, ac, that increased with the tissue stress, a (FIGS. 9(A)-(B)).

FIG. 10(A) shows in stretched tissues, the strain εₘ in the ECM increases over time due to ECM viscous nature. FIG. 10(B) illustrates that over longer stretches (e.g., 24 h), ECM can be stretched beyond the point at which the ECM softens. The dashed lines in FIG. 9(D) represent an average passive constitutive behavior of tissues from the experiments. FIG. 10(C) shows the effect of tissue strain magnitude, ε. At a strain sufficiently high to cause this ECM softening, both the stress generated in the tissue, σ, and the cell stress, σ_{c}, drop significantly. On the contrary, shorter durations of strain(1 h), are insufficient for creep to lead to ECM softening, so both the stress generated in the tissue σ and the stress that cells experience σ_{c} are predicted to increase monotonically with the tissue strain magnitude c. FIGS. 10(D)-(F) show combining these cell-level predictions with a cell signaling model enabled prediction of a phase diagram that captured the biphasic activation of cells. As shown in FIGS. 10(G)-(I), the integrated model thus can predict the seemingly contradictory observations in activation of cell contractility and tissue contraction force as a function of the magnitude and duration of tissue stretch.

In a tissue stretched and held isometrically at strain ε, the ECM is stretched in tension by both cell-generated contractile stresses (σ_{c}) and the external stresses applied to maintain the tissue strain of ε (FIG. 10(A)). However, due to the viscous nature of the ECM, the tensile strain in the matrix, εₘ, increases with the duration of tissue stretching when the tissue is stretched isometrically (FIG. 10(A)). As a result, for long stretching durations (e.g., 24 h), the strain in the ECM can locally exceed the strain associated with peak stress in FIG. 9(D). This effect could be seen in the simulations: after 1 h of isometric tissue stretching, the ECM strain stayed sufficiently low that the slope of the stress-strain curve (Etangent) was independent of tissue strain magnitude ε, but after 24 h, the ECM strain reached or passed the peak so that E tangent dropped and varied with ε (FIG. 10(B)). To verify the model predictions against the experiments in FIG. 8(E), ECM stiffness (k) was calculated. It was found that the model prediction is in close agreement with those from the experiments in FIG. 8(E)(FIG. 8(F)).

The model also provided a mechanism for how changes in ECM stiffness affect the tension generated in the tissues, σ, and subsequently the cell stress σ_{c} (FIG. 10(C)). For 1 h of tissue stretching, the ECM strain remained below the value associated with peak stress so that the stress generated in the tissue σ increased monotonically with the tissue strain magnitude ε (FIG. 10(C)). However, for 24 h of tissue stretching, the strain in the ECM passed the peak, softening the ECM. This effect increased with tissue strain magnitude ε such that for high ε, the stress in the tissue, σ, dropped, which in turn decreased cell-generated stress σ_{c} (FIG. 10(C)). Taken together, results showed that time- and strain-dependent changes in the ECM stiffness alter the stress generated in a stretched tissue, which in turn affects the stress level that cells experience.

Increasing magnitude and duration of stress can increase or decrease the long-term activation level of cells.

A signaling network model was developed (FIG. 10(D)) and coupled it to the active chemo-mechanical tissue model to interpret data for how the magnitude and duration of the stress on cells in stretched tissues affected the long-term activation of cells that persisted after the tissues were unloaded. The signaling network model incorporated the following cascade of biological processes.

First, tensile stress promoted cell actomyosin contractility through pathways such as Rho-Rock or activation of mechanosensitive ion channels (part (i) of FIG. 10(D)). While excessive cell contraction can cause detachment of the cell from the extracellular matrix, thereby switching cells to a transcriptionally less active state (parts v, viii of FIG. 10(D)), modest increases in cell contraction led to translocations of transcriptional and epigenetic factors (e.g., MKL and YAP/TAZ) to the nucleus (part iv of FIG. 10(D)), which in turn promotes transcription of genes associated with actomyosin contractility and cell activation (parts vi, vii of FIG. 10(D)). Finally, expression of these genes increases cell actomyosin contractility which is captured in the model through a feedback loop (part ii, iii of FIG. 10(D)).

The input to the signaling network model was the time variation of cell stress during tissue stretching, as predicted by the active chemo-mechanical tissue model (FIG. 10(C)). The output was the level of cellular actomyosin contractility that persisted after releasing the tissues. Note that cell stress ø_{c} was relatively high in tissues pre-strained for 24 h, displaying a monotonic increase with tissue strain c (FIG. 10(C)). Conversely, cell stress was relatively low in tissues pre-strained for 1 h and exhibited a biphasic trend with tissue strain (FIG. 10(C)). The result was that cells in tissues pre-strained for 24 h with relatively low stretch were predicted to retain a higher state of activation than cells in tissues pre-strained for 24 h with relatively high stretch (FIGS. 10(E)-(F), 11(C)). Cells in tissues pre-strained for 1 h, even for cases of high tension magnitude, were not predicted to retain high levels of activation (FIGS. 10(E)-(F), 11(C)).

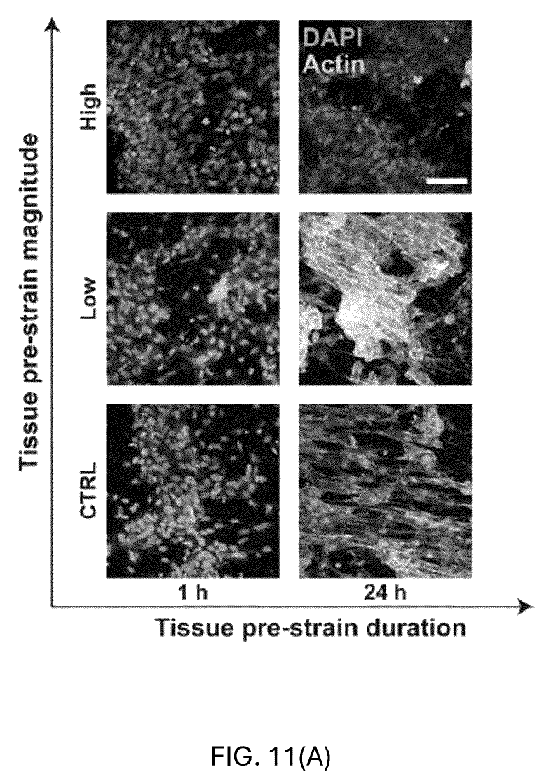

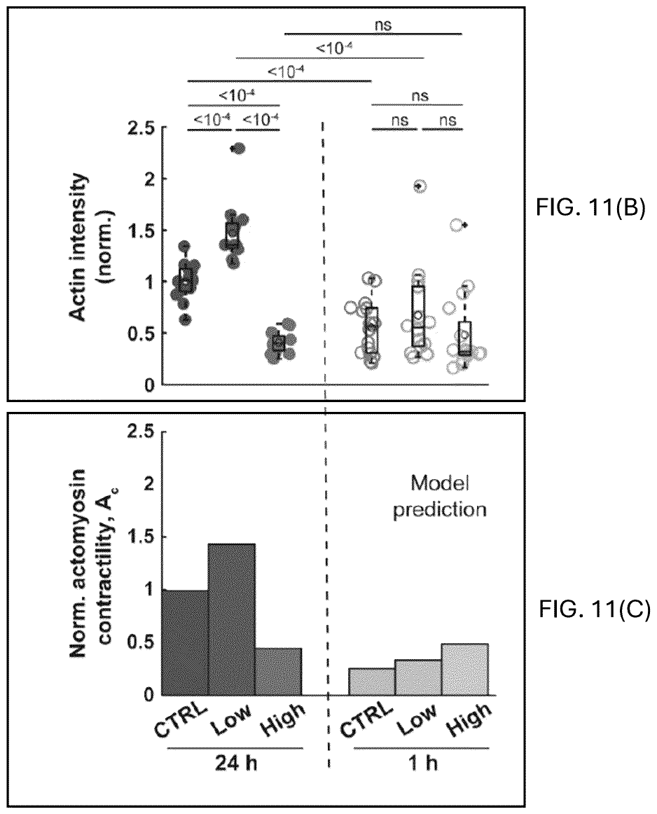

These cell-level model predictions were counterintuitive, given the diametrically opposite experimental observation at the tissue level, with pre-strained tissues for 24 h having significantly lower contractility than tissues pre-strained for 1 h. To verify the cell-level model predictions, tissues were fixed one day after tissue unloading and stained for F-actin (FIGS. 11(A)-(B) and α-SMA which are indicators of the level of fibroblast activation. Results were consistent with model predictions, and supported the hypothesis that long-term activation of cells that reside within a tissue is modulated by the way that force is conducted to these cells through the ECM.

Long-term tissue contraction force depends on both cell activation level and matrix stiffness after tissue unloading.

A seeming contradiction is that cells in tissues stretched for 24 h in control or low stretch conditions had higher sustained levels of activation than cells in tissues stretched for 1 h (FIGS. 11(A)-(C), but tissue-level contractile forces for these tissues stretched for 24 h were remarkably close to those for tissues stretched for 1 h (FIG. 7(E)). The present theoretical framework identified that the mechanism underlying this is the transmission of force from cells by the ECM. In the tissues stretched for 24 hours, the ECM softens (FIGS. 8(E), 8(F)), reducing the tissue-level stress associated with cell contraction. This is shown in FIG. 7(F) where the theoretical framework is used to determine the tissue contractile stress (a) 1 day after tissue unloading (and not during the tissue stretching as in FIG. 10(C)). Model predictions showed good agreement with the experiments (FIG. 7(E)), and revealed that elevated cell activation and cell contractility do not necessarily lead to elevated tissue contraction because ECM stiffness modulates the transmission of cellular forces. The ECM softening associated with long-duration stretching attenuates the transmission of cell contractile forces to the tissue level (FIGS. 10(G)-(I)).

FIGS. 11(A)-(C) illustrate stretching of skin tissues for a long duration can induce mechanical memory in cells and cause their long-term activation in a non-monotonic manner with tissue strain magnitude. FIGS. 11(A)-(B) show to determine the long-term level of cell activation, tissues were fixed 1 day after tissue unloading and the expression levels of F-actin in fibroblasts per cell were measured. F-actin is an indicator of fibroblast activation level. Scale bar=50 μm. n≥14 for both 24 h and 1 h groups. FIG. 11(C) shows the cell activation level induced by the magnitude and duration of tension is predicted by the signaling network model.

Using an integrated experimental and theoretical approach, the present investigators described mechanisms by which cell-ECM feedback modulates both the long-term activation of cells and tissue contractility. This occurred at strain levels known to be important for wound healing and skin grafting. Unlike previous observations on stem cells, fibroblasts developed mechanical memory under static tissue strain, depending on the duration and magnitude of tissue strain. Central to this was a role of the ECM in the development of mechanical memory in fibroblasts and their long-term activation, modulated by time- and strain-dependent physical properties of the ECM.

### Materials and Methods: Cell Culture and 3d Tissue Construction

Human dermal fibroblasts (HDFs), derived from adult dermis, were purchased from Lonza (Basel, Switzerland, Catalog #: CC-2511). Cells were used only at passages P4 to P8 in the experiments. Cells were first cultured at 37° C. with 5% CO2 in Dulbecco's Modified Eagle Medium (DMEM) containing 10% fetal bovine serum (FBS) and 1% penicillin-streptomycin (PS). Cells were next detached with 0.05% Trypsin/EDTA for 5 min, separated with Accutase (Sigma Aldrich, St. Louis, MO), and resuspended in fresh DMEM. The suspended cells in DMEM were mixed with rat tail-derived type I collagen in 0.1% acetic acid and 2×DMEM (Sigma). After neutralizing the pH with sodium hydroxide, 1 mL of the mixture containing 1.5×106 cells/mL HDFs and 1.2 mg/mL collagen was added into Teflon-made cylindrical casting molds with a mandrel at the center with a diameter of 9.65 mm (FIG. 7(A)). The tissues were incubated at 37° C. with 5% CO2 for 2 days and formed compacted and thin contractile rings with cells aligned in the circumferential direction of the molds.

### Tissue Force Measurement Experiments

The contractile force was measured in each tissue before ((F⁰)ₚᵣₑ: part (i) of FIG. 7(C) and after ((F⁰)ₚₒₛₜ: part (iv) of FIG. 7(C) tissue stretching. The tissue force measurements were performed using a custom-made tensile tester with sensitive isometric force transducers (Harvard Apparatus) and stepper motors (Haward Industry) plugged into an Arduino for applying displacement precisely (FIG. 7(B)). A MATLAB subscript was developed for collecting the output voltages reported by the transducers, which were calibrated to the force. To control the temperature, a water pump with a temperature control system was connected to the organ bath (FIG. 7(B)).

The tissue rings were removed from the casting molds after 2 days of compaction (FIG. 7(A)) and were hung onto the attachment bars, bathed with 25 mM HEPES buffered DMEM with 10% FBS and 1% PS at 37° C. (FIG. 7(B)). The attachment bars were then separated at a 13.3 mm distance under the control of the Arduino (part (i) of FIG. 7(C)). At this length, the periphery of the tissues on the stretcher device is equal to the periphery of the tissues in the mold on day 2. The tissue force was measured by lifting the lower attachment bars away from the samples to define the zero-force point and bring it back to the 13.3 mm distance. The average of forces measured during the first 200 s of force measurement was reported as the tissue contractile force.

After measuring the initial tissue contractile force, the tissues were moved back to the incubator and were kept in sodium bicarbonate buffered DMEM at 37° C. with 5% CO2. 3D-printed spacers were designed with different lengths and placed between the two ends of the tissues to generate 0% (tissue length=13.3 mm), 5%, and 30% strains in the tissues (part (ii) of FIG. 7(C)). The tissues were kept under the prescribed strain levels either for 1 hour or 24 hours (part (ii) of FIG. 7(C)). The spacers were then removed and the tissues were allowed to relax for 1 day (part (iii) of FIG. 7(C)). The contractile force was measured in each tissue using the force measurement procedure described above (part (iv) of FIG. 7(C)).

### Stress Relaxation Test

After measuring the contractile force of pre-strained tissues (part (iv) of FIG. 7(C)), tissues were allowed to relax for 10 minutes and then were hung onto the attachment bars of the tensile tester. 5% strain was applied to the tissues. The tissues were kept under the strain for 10 minutes (part (v) of FIG. 8(A)) while continuously measuring and recording the force (FIG. 8(B)). The stiffness of the tissue was measured as ΔF/ΔX where ΔF is the difference between forces measured at t=10 min and t=0, respectively, and ΔX=(0.05)(13.3 mm) is tissue deformation.

The tissues were allowed to relax for 10 minutes and were then treated with 2 μM cytochalasin-D for 1 hour to disrupt the cell-generated contractile forces (part (vi) of FIG. 8(A)). The stress relaxation test was repeated (part (vii) of FIG. 8(A)) to measure the stiffness as described above (FIG. 8(E)).

### Matrix Stress-Strain Relationship

After the stress relaxation test on cytochalasin-D treated tissues, the tissues were relaxed for 10 minutes on the tensile tester. Starting from the initial tissue length of 13.3 mm, the tissues were then stretched with a loading rate of 3.11 μm/s to determine the force-strain curves in FIG. 9(D). Note that, as depicted in FIG. 8(E), tissues pre-strained for 24 h showed ECM softening, while tissues pre-strained for 1 h did not exhibit permanent changes in their long-term ECM stiffness. Therefore, for the determination of the matrix stress-strain relationship depicted in FIG. 3D, only tissues pre-strained for 1 hour were used.

### Immunofluorescence And Cell Imaging In 3D Collagen Gels

To determine the long-term level of cell activation, the tissues were fixed one day after tissue unloading, and cells were stained for F-actin (FIGS. 11(A)-(B) and α-SMA. Tissues were fixed with 4% paraformaldehyde (Electron Microscopy Sciences, Catalog #: 15714) for 2 hours at 37° C., permeabilized with 0.5% triton X-100 solution (Sigma, X100) for 30 minutes and blocked with 10% goat serum (Thermos Fisher, Catalog #: 50062Z) for 1 hour at room temperature before incubating with anti-α-smooth-actin antibody (1:250 dilution, Abcam, Catalog #: ab124964) in 10% goat serum at 4° C. overnight. Tissues were incubated with rhodamine-phalloidin (1:400 dilution, Thermos Fisher, Catalog #: R415) with Alex Fluor 488-conjugated secondary antibody (1:500 dilution, Abcam, Catalog #: ab150077) in 10% goat serum for 2 hours at room temperature. Each step was followed by PBS washing for a minimal 10 minutes at least 3 times. Samples were mounted to glass-bottom dishes with a prolonged gold antifade mounting solution with DAPI (Thermos Fisher, Catalog #: P36931). Z-stack images were obtained through a Zeiss LSM 880 laser confocal microscope with identical parameters in each experiment.

### Skin Graft Strain Field

The Holzapfel-Gasser-Ogden model was used to determine the average strain magnitude in stretched skin grafts. The model was first fitted to unmeshed skin stretching experiments to determine the model parameters. Then, the calibrated model was used to simulate the stretching of a meshed skin graft with meshing ratios of 1.3, 3, and 5, commonly used in skin graft surgeries.

Active chemo-mechanical tissue model and cell signaling network model. A full description of model parameters and computer codes are available on GitHub (https://github.com/Farid-Alisafaei/Stretch-Induced-Cellular-Memory.git) incorporated by reference, herein.

### Possible Variations

There are several features or steps of the present innovation that could potentially be varied or substituted while maintaining the core inventive concept of optimizing skin graft expansion. Some possibilities include, but are not limited to, the cutting mechanism. The specific type of cutting mechanism could be varied (e.g., laser cutting, water jet cutting, or mechanical blade systems) while maintaining the ability to create precise, complex patterns. The sensor types may be varied. Different types of sensors could be used to measure skin properties and cutting forces to provide real-time data to inform the cutting process. The user interface may be varied. The method of interacting with the device (e.g., touchscreen, voice commands, or gesture controls) could be varied without affecting the core functionality. Data logging system may be altered.

The specific method of recording and storing procedure data could be modified to also allow for analysis and improvement of the process over time. The tissue support mechanism may be varied. The method of holding and tensioning the graft during cutting could be varied and also allow for precise control during the cutting process. Pattern generation via the software may be varied for generating cutting patterns implemented in various ways, and also produce both conventional and kirigami-inspired patterns based on input parameters.

Variations in the integration with existing systems may be accomplished. The present device and method may be designed to integrate with different types of existing surgical equipment or hospital information systems. The materials used for the device's construction, particularly for parts in contact with the graft, may vary while maintaining biocompatibility and the necessary mechanical properties.

Expansion measurement may be varied. The present device and method utilize distinct pattern geometry. The patterns created by the present device and methods are distinct from traditional parallel cuts. The present device and method are also designed to achieve specific expansion ratios that conventional devices fail to deliver. The method of measuring and verifying the achieved expansion ratio and tension could potentially be varied (e.g., optical measurement, mechanical measurement, or image analysis).

Any headings and sub-headings utilized in this description are not meant to limit the aspects described thereunder. Features of various aspects described herein may be utilized with other aspects even if not described under a specific heading for that aspect.

Although the disclosure herein has been described with reference to particular aspects, it is to be understood that these aspects are merely illustrative of the principles and applications of the present disclosure. It is therefore to be understood that numerous modifications may be made to the illustrative aspects and that other arrangements may be devised without departing from the spirit and scope of the present disclosure as defined by the appended claims.

While exemplary aspects have been described herein, it is expressly noted that these aspects should not be construed as limiting, but rather that additions and modifications to what is expressly described herein also are included within the scope of the disclosure. Moreover, it is to be understood that the features of the various aspects described herein are not mutually exclusive and can exist in various combinations and permutations, even if such combinations or permutations are not made express herein, without departing from the spirit and scope of the disclosure.

1. A method for optimizing skin graft expansion in split-thickness skin graft surgeries, comprising:
selecting, from an integrated system, a desired expansion ratio for a specific skin graft expansion in a patient;
receiving, by the integrated system, an input of a set of geometric constraints;
determining, by the integrated system, a first set of cutting patterns based on the desired expansion ratio and the input set of geometric constraints, wherein the first set of cutting patterns satisfies both the desired expansion ratio and the set of geometric constraints; and
outputting, by the integrated system, the first set of cutting patterns, the first set of cutting patterns including a cutting pattern including one or more of a parallel cutting pattern or a complex geometrical cutting pattern.

2. The method of claim 1, wherein the determining of the first set of cutting patterns includes selecting the first set of cutting patterns from a larger set of cutting patterns stored in the integrated system, the selecting of the first set of cutting patterns being based on which of the larger set of cutting patterns satisfies both the desired expansion ratio and the set of geometric constraints.

3. The method of claim 2, wherein the larger set of cutting patterns are selected prior to the selecting of the desired expansion ratio.

4. The method of claim 3, wherein the larger set of cutting patterns are stored in a memory of the integrated system.

5. The method of claim 2, wherein the larger set of cutting patterns are selected by excluding possible cutting patterns that: result in geometric overlap between adjacent cuts, exceed predefined limits on spacing between cuts, have fewer than a minimum number of cuts in a horizontal and a vertical direction, and are unable to achieve the desired expansion ratio.

6. The method of claim 5, wherein the geometric overlap between adjacent cuts is determined using a cut length L, a horizontal spacing between cuts d, and a vertical gap between cut ends g.

7. The method of claim 6, wherein the selecting of the desired expansion ratio R from the integrated system for the specific skin graft expansion in the patient comprises determining R using the following Eq. 2:
$$R = \frac{1}{2} ( \frac{1 + λ}{1 + γ} ) [ 2 γ + \sqrt{{( 1 - γ )}^{2} - {ϕ ( λ - 1 )}^{2} {η}^{2}} ] \qquad ( 2 )$$
wherein λ=1+(δ/d), γ=g/L, and η=d/L.

8. The method of claim 7, wherein an upper limit for the extension ratio R_{U} is determined using the following Eq. 3:
$${R}_{U} = \frac{1}{2} ( 1 + λ ) \qquad ( 3 )$$

9. The method of claim 8, wherein a lower limit for the extension ratio R_{L} is determined using the following Eq. 4:
$${R}_{L} = \{ \begin{matrix}\frac{1}{2} ( \frac{1 + λ}{1 + γ} ) [ 2 γ + \sqrt{{( 1 - γ )}^{2} - {( λ - 1 )}^{2} {η}^{2}} ] , & λ \leq {λ}_{c} \\ \frac{γ ( 1 + λ )}{1 + γ} , & λ > {λ}_{c}\end{matrix} \qquad ( 4 )$$

10. The method of claim 9, wherein a limit of elongation λₐ is determined using the following Eq. 5:
$${λ}_{a} = 1 + \frac{k}{η} \sqrt{2 ( 1 - {γ}^{2} ) - {{k}^{2} ( 1 + γ )}^{2}} \qquad ( 5 )$$
wherein R_{U}−R_{L}=k²R_{U}.

11. The method of claim 10, wherein an actual expansion ratio Rₐ is determined using the following Eq.6:
$${R}_{a} = \frac{1}{2} ( 1 + {λ}_{a} ) \qquad ( 6 )$$

12. The method of claim 1, further comprising initiating a cutting process with a device, wherein the device comprises an adaptive cutting mechanism having a multi-modal cutting head and a force-feedback sensor integrated into the cutting mechanism to monitor a resistance during cutting for real-time adjustments to a cutting depth and a cutting speed.

13. The method of claim 12, further comprising placing a harvested graft in the device, wherein the device further comprises a microfluidic tissue support that uses micro-fluidics to maintain a specific position and a tension of the harvested graft during the cutting process of the parallel cutting pattern or the complex geometrical cutting pattern without damaging tissue.

14. The method of claim 13, further comprising expanding the harvested graft and receiving from the integrated system a real-time feedback on achieved expansion ratio as the harvested graft is expanded.

15. The method of claim 14, wherein the real-time feedback on the achieved expansion ratio further comprises providing an alert when the harvested graft approaches a selected expansion point, and wherein the selected expansion point is based on a predictive model and sensor data.

16. The method of claim 1, further comprising logging data including parameters recorded by the integrated system of a skin grafting procedure for future reference and analysis.

17. The method of claim 1, further comprising conducting a post-procedure analysis wherein the integrated system provides a summary of a skin grafting procedure including an actual expansion ratio and deviations from predictions.

18. The method of claim 1, wherein the complex geometric cutting pattern includes kirigami-inspired cuts.

19. The method of claim 13, further comprising adjusting the device in real-time using the resistance monitored by the force-feedback sensor during the cutting process.

20. The method of claim 19, wherein the adjustment of the device further comprises allowing the integrated system to adjust the cutting depth and the cutting speed in real-time.
